(19) United States
(12) Patent Application Publication
(10) Pub. No.: US 2005/0113283 A1
(43) Pub. Date: May 26, 2005

(54) Methods of treating conditions associated with an EDG-4 receptor

(76) Inventors: David Solow-Cordero, San Francisco, CA (US); Geetha Shankar, Menlo Park, CA (US); Juliet Spencer, San Mateo, CA (US); Charles Gluchowski, Danville, CA (US)

Correspondence Address: MORGAN LEWIS & BOCKIUS LLP, 1111 PENNSYLVANIA AVENUE NW, WASHINGTON, DC, 20004, US

(21) Appl. No.: 10/390,429

(22) Filed: Mar. 14, 2003

Related U.S. Application Data
(63) Continuation-in-part of application No. 10/347,182, filed on Jan. 21, 2003, now abandoned.
(60) Provisional application No. 60/350,445, filed on Jan. 18, 2002.
(60) Provisional application No. 60/438,996, filed on Jan. 10, 2003.
(60) Provisional application No. 60/440,335, filed on Jan. 16, 2003.
(60) Provisional application No. 60/440,332, filed on Jan. 16, 2003.
(60) Provisional application No. 60/440,329, filed on Jan. 16, 2003.
(60) Provisional application No. 60/440,346, filed on Jan. 16, 2003.
(60) Provisional application No. 60/440,347, filed on Jan. 16, 2003.
(60) Provisional application No. 60/440,334, filed on Jan. 16, 2003.
(60) Provisional application No. 60/440,331, filed on Jan. 16, 2003.
(60) Provisional application No. 60/440,345, filed on Jan. 16, 2003.
(60) Provisional application No. 60/440,328, filed on Jan. 16, 2003.

(51) Int. Cl. A61K 31/00

(52) U.S. Cl. 514/1

(57) ABSTRACT

The present invention provides a method of modulating an Edg-4 receptor mediated biological activity in a cell. A cell expressing the Edg-4 receptor is contacted with a modulator of an Edg-4 receptor sufficient to modulate the Edg-4 receptor mediated biological activity. In another aspect, the present invention provides a method for modulating an Edg-4 receptor mediated biological activity in a subject. A therapeutically effective amount of a modulator of the Edg-4 receptor is administered to the subject.

This is a continuation-in-part of U.S. patent application Ser. No. 10/347,182, filed Jan. 17, 2003, which is entitled to and claims priority to U.S. Provisional Application No. 60/350,445, filed Jan. 18, 2002, each of which is hereby incorporated by reference in its entirety.

### 1. FIELD OF INVENTION

The present invention relates generally to methods of modulating biological activity mediated by the Edg-4 receptor. More specifically, the present invention provides compounds and compositions, which may be used to selectively modulate, e.g., antagonize the Edg-4 receptor. The present invention also provides methods for making these compounds.

### 2. BACKGROUND OF THE INVENTION

Recent studies have revealed a complex biological role for cell membrane phospholipids, which were previously believed to have only a structural function. Following cell activation, membrane phospholipids may be metabolized to eicosanoids and lysophospholipids, which are important regulators of cellular function and behavior. Lysophospholipids include compounds such as lysophosphatidic acid (“LPA”), sphingosine-1-phosphate (“S1P”), lysophosphatidylcholine and sphingosylphosphorylcholine and are important second messengers that can activate particular cell surface transmembrane G-protein coupled receptors known as endothelial gene differentiation (“Edg”) receptors.

Two quite distinct subfamilies of GPCRs bind LPA and S1P specifically and transduce diverse cellular signals by associating with one or more G proteins. Based on amino acid sequence identities, S1P1 (Edg 1), S1P3 (Edg 3), S1P2 (Edg 5), and S1P5 (Edg 8) belong to one structural cluster and LPA1 (Edg 2), LPA2 (Edg 4) and LPA3 (Edg 7) are members of a second structural cluster (Goetzl, B. J., and Lynch, K. R. 2000, Ann. N. Y Acad. Sci. 905:1-357). Members of both subfamilies range in size from 351 to 400 amino acids, and are encoded by chromosomes 1, 9 or 19. The amino acid sequence of S1P4 (Edg 6) lies between those of the two major clusters by amino acid sequence identity (Graler et al, 1998, Genomics 53, 164-169). Edg-6, a novel G-protein-coupled receptor related to receptors for bioactive lysophospholipids, is specifically expressed in lymphoid tissue. (Graler et al, 1998, Genomics 53, 164-169). Currently, there are three known Edg receptors specifically activated by LPA (LPA1 or Edg 2, LPA2 or Edg 4 and LPA3 or Bdg 7) and five known S1P receptors specifically activated by S1P (S1P1 or Edg 1, S1P2 or Edg 5, S1P3 or Edg 3, S1P4 or Edg 6, and S1PS or Edg 8).

Edg-1 (human Edg-1, GenBank Accession No. AF233365), Edg-3 (human Edg-3, GenBank Accession No. X83864), Edg-5 (human Edg-5, GenBank Accession No. AF034780), Edg-6 (human Edg-6, GenBank Accession No. AJ000479) and Edg-8 (human Edg-8, GenBank Accession No. AF3 17676) receptors are activated by S1P, while LPA activates Edg-2 (human Edg-2, GenBank Accession No., U78 192), Edg-4 (human Edg-4, GenBank Accession Nos. AF233092 or AFO1 1466) and Edg-7 (human Edg-7, GenBank Accession No. AF127 138) receptors. Although, all three LPA receptors (i.e., Edg-2, Edg-4 and Edg-7) bind LPA, compounds, which discriminate between these receptors have been identified (Im et al, 2000, Mol. Pharmacol. 57 (4):753-759). Further, Edg 2, Edg-4 and Edg-7 appear to exhibit significant pharmacological differences (Bandoh et al., 2000, FEBS Lett. 478:159-165).

Importantly, Edg receptors are believed to mediate critical cellular events such as cell proliferation and cell migration, which makes these receptors attractive therapeutic targets. However, currently known compounds, which bind to LPA, are almost exclusively phospholipids (e.g, LPA and S1P, analogs of LPA and S1P, dioctyl glycerol, etc). Most of these phospholipids compounds fail to effectively discriminate between different Edg receptors and have poor physicochemical properties, which limits their potential use as pharmaceutical agents. Thus, there exists a need for compounds, which are not phospholipids that bind or otherwise regulate Edg receptors and can also selectively bind to a specific Edg receptor.

### 3. SUMMARY OF THE INVENTION

The present invention addresses these and other needs by providing compounds that modulate the Edg-4 (LPA2) receptor (e.g, human Edg-4, GenBank Accession Nos. AF233092 or AFO1 1466). Such compounds preferably selectively bind or otherwise modulate the Edg-4 receptor.

The present invention provides methods for modulating (antagonizing or agonizing) Edg-4 receptor mediated biological activity. The present invention also provides methods for using Edg-4 modulators (antagonists or agonists) in treating or preventing diseases such as ovarian cancer, peritoneal cancer, endometrial cancer, cervical cancer, breast cancer, colorectal cancer, uterine cancer, stomach cancer, small intestine cancer, thyroid cancer, lung cancer, kidney cancer, pancreas cancer and prostrate cancer; acute lung diseases, adult respiratory distress syndrome (“ARDS”), acute inflammatory exacerbation of chronic lung diseases such as asthma, surface epithelial cell injury, (e.g., transcorneal freezing or cutaneous bums) and cardiovascular diseases (e.g., ischemia) in a subject in need of such treatment or prevention. Further, the present invention provides compounds and compositions that can, for example, be used in modulating Edg-4 receptor mediated biological activity or treating or preventing diseases such as those mentioned above. The present invention still further provides methods for synthesizing the compounds.

In one aspect, the present invention provides a method of modulating an Edg-4 receptor mediated biological activity in a cell. A cell expressing the Edg-4 receptor is contacted with an amount of an Edg-4 receptor modulator sufficient to modulate the Edg-4 receptor mediated biological activity.

In another aspect, the present invention provides a method for modulating Edg-4 receptor mediated biological activity in a subject. In such a method, an amount of a modulator of the Edg-4 receptor effective to modulate the Edg-4 receptor mediated biological activity is administered to the subject.

The present invention also provides compounds (agonists or antagonists) that modulate Edg-4 receptor mediated biological activity. The agonists or antagonists are compounds of structural formula (I) and can be utilized as part of the methods of the present invention: 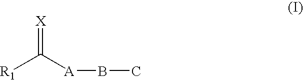
or a pharmaceutically available solvate or hydrate thereof, wherein:

R₁ is hydrogen, alkyl, substituted alkyl, acylamino, substituted acylamino, alkylamino, substituted alkylamino, alkylthio, substituted alkylthio, alkoxy, substituted alkoxy, alkylarylamino, substituted alkylarylamino, amino, arylalkyloxy, substituted arylalkyloxy, aryl, substituted aryl, arylamino, substituted arylamino, arylalkyl, substituted arylalkyl, dialkylamino, substituted dialkylamino, cycloalkyl, substituted cycloalkyl, cycloheteroalkyl, substituted cycloheteroalkyl, heteroaryloxy, substituted heteroaryloxy, heteroaryl, substituted heteroaryl, heteroalkyl, substituted heteroalkyl sulfonylamino or substituted sulfonylamino;

X═O or S;

A is NR₂, O or S;

R₂ is hydrogen, alkyl or substituted alkyl; and

B and C are independently alkyl, substituted alkyl, aryl, substituted aryl, heteroaryl, substituted heteroaryl, cycloalkyl, substituted cycloalkyl, cycloheteroalkyl or substituted cycloheteroalkyl.

In another embodiment, the agonists or antagonists that can be utilized as part of the methods of the present invention are compounds of structural formula (IV): 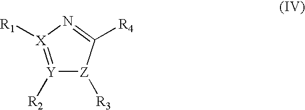
or a pharmaceutically available solvate or hydrate thereof, wherein;

each of R₁, R₂, R₃, R₄ or R₅ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(CH₂)ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-₁₀)cycloalkenyl, —(C₅)heteroaryl, —(C₆)heteroaryl, —(C₅-C₁₀)heteroaryl, —-(C₅-C₁₀)cycloheteroaryl, —(C₃-C₆)cycloheteroalkyl, -naphthyl, —(C₃-C₁₀)heterocycle, —CO₂(CH₂)ₘR₅, —NHC(O)R₅, —NHC(O)OR₅, —NHC(O)NHR₅, —NR₅R₅, ═NR₅, —(C₁-C₁₀)alkylNHC(O)(CH₂)ₘR₅, —(C₃-C₁₀)cycloheteroalkyl(R₅)ₘ, —(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 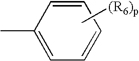
wherein;

each R₅ and R6 is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl (C₁-₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

X and Y are each independently C or N; and

Z is O, S, C or N, wherein if Z is O or S, then R₃ is an electron pair;

R₁ and R₂ can optionally together form a 5-, 6-, or 7-membered substituted or unsubstituted cyclic or aromatic ring;

R₂ and R₃ can optionally together form a 5-, 6-, or 7-membered substituted or unsubstituted cyclic or aromatic ring; and

R₃ and R₄ can optionally together form a 5-, 6-, or 7-membered substituted or unsubstituted cyclic or aromatic ring.

In another embodiment, the modulator is a compound of structural formula (V): 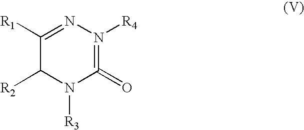
or a pharmaceutically available solvate or hydrate thereof, wherein;

each of R₁, R₂, R₃, R₄ or R₅ is independently —H, -halo, —NO₂, —CN, —OH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —OC(O)(CH₂)ₘCHR₅R₅, —CO₂(CH₂)ₘCHR₅R₅,—OC(O)OR₅, —SR₅, —S(O)R₅, —S(O)₂R₅, —S(O)₂NHR₅, or 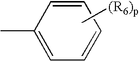
wherein;

each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)(C₁-C₁₀)alkyl, —OC(O)O(C₁-C₁₀)alkyl, or —SO₂NH₂;

m is independently an integer ranging from 0 to 8;

p is independently an integer ranging from 0 to 5; and

R₁ and R₂ or R₂ and R₃ can optionally together form a 5-, 6-, or 7-membered substituted or unsubstituted cyclic or aromatic ring.

In yet another embodiment, the agonists or antagonists are compounds of structural formula (VI): 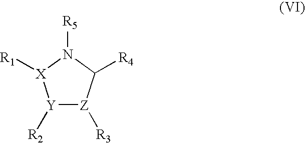
or a pharmaceutically available solvate or hydrate thereof, wherein;

each of R₁, R₂, R₃, R₄ or R₅ is independently —H, -halo, —NO₂, —CN, —OH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂—C₁₀)alkenyl, —(C₂—C₁₀)alkynyl, —(C₃—C₁₀)cycloalkyl, —(C₈-C₁₄)bicycloalkyl, —(C₅-C₁₀)cycloalkenyl, —(C₅)heteroaryl, —(C₆)heteroaryl, -indole, -naphthyl, —(C₃-C₁₀)heterocycle, —CO₂(CH₂)ₘR₅, —NHC(O)R₅, —NHC(O)OR₅, —NHC(O)NHR₅, —OC(O)(CH₂)ₘCHR₅R₅, —CO₂(CH₂)ₘCHR₅R₅,—OC(O)OR₅, —SR₅, —S(O)R₅, —S(O)₂R₅, —S(O)₂NHR₅, or 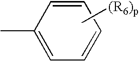
wherein;

R₅ or R₆ is independently -halo, —NO₂, —CN, —OH, —CO₂H, —N(C —C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)(C₁-C₁₀)alkyl, —OC(O)O(C₁-C₁₀)alkyl, or —SO₂NH₂;

m is independently an integer ranging from 0 to 8;

p is independently an integer ranging from 0 to 5;

X, Y and Z are independently O, S, C or N, wherein if X, Y or Z is O or S, R₁ is an electron pair;

R₁ and R₂ can optionally together form a 5-, 6-, or 7-membered substituted or unsubstituted cyclic or aromatic ring;

R₃ and R₄ can optionally together form a 5-, 6- or 7-membered substituted or unsubstituted cyclic or aromatic ring;

R₁ and R₅ can optionally together form a 5-, 6- or 7-membered substituted or unsubstituted cyclic or aromatic ring; and

R₄ and R₅ can optionally together form a 5-, 6- or 7-membered substituted or unsubstituted cyclic or aromatic ring.

In another embodiment, the agonists or antagonists that can be utilized as part of the methods of the present invention are compounds of structural formula (VII): 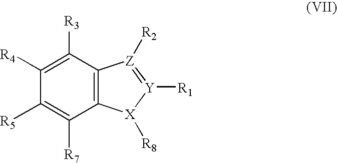
or a pharmaceutically available solvate or hydrate thereof, wherein;

each of R₁, R₂, R₃, R₄, R₅, R₇ or R₈ is independently —H, -halo, —NO₂, —CN, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂—C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅—Cl₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 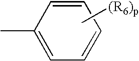
wherein;

each R₅ or R6 is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂—C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

X is O, S, C or N, wherein if X is O or S, R₁ is an electron pair; and

Y and Z are independently N or C, wherein if Y or Z is N, R₁ and R₂ are each an electron pair.

In another embodiment, the agonists or antagonists that can be utilized as part of the methods of the present invention are compounds of structural formula (VIII): 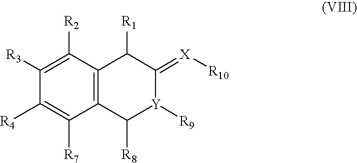
or a pharmaceutically available solvate or hydrate thereof, wherein;

each of R₁, R₂, R₃, R4, R₅, R₇, R₈, R₉ or R₁₀ is independently —H, -halo, —NO₂, —CN, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂—C₁₀)alkynyl, —(C₃-C₁₀)cycloalkyl, —(C₈-C₁₄)bicycloalkyl, —(C₅—C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 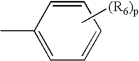
wherein;

each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5; and

X and Y are independently O, S or N, wherein if X or Y is O or S, R₉ and R₁₀ are an electron pair.

In yet another embodiment, the agonists or antagonists that can be utilized as part of the methods of the present invention are compounds of structural formula (IX): 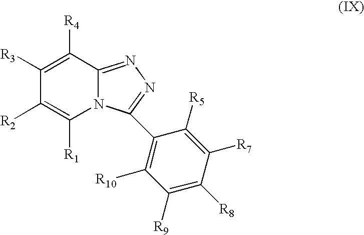
or a pharmaceutically available solvate or hydrate thereof, wherein;

each of R₁, R₂, R₃, R₄, R₅, R₇, R₈, R₉ or R₁₀ is independently —H, -halo, —NO₂, —CN, —C(R₅)₃, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃—C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 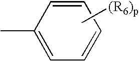
wherein;

each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂—C₁₀)alkynyl, —(C₃-C₁₀)cycloalkyl, —(C₈-C14)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8; and

p is independently an integer ranging from 0 to 5.

In yet another embodiment, the agonists or antagonists that can be utilized as part of the methods of the present invention are compounds of structural formula (X): 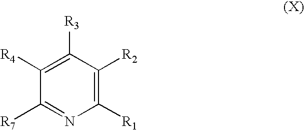
or a pharmaceutically available solvate or hydrate thereof, wherein;

each of R₁, R₂, R₃, R₄, R₅ or R₇ is independently —H, -halo, —NO₂, —CN, —C(R₅)₃, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂—C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —CO₂H, —(C₁-C₁₀)alkylC(O)NH(CH₂)ₘR₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 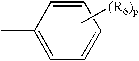
wherein;

each R₅ or R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂—C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

R₁ and R₂ can optionally together form a 5-, 6- or 7-membered substituted or unsubstituted cyclic or aromatic ring;

R₂ and R₃ can optionally together form a 5-, 6- or 7-membered substituted or unsubstituted cyclic or aromatic ring;

R₃ and R4 can optionally together form a 5-, 6- or 7-membered substituted or unsubstituted cyclic or aromatic ring; and

R₄ and R₇ can optionally together form a 5-, 6- or 7-membered substituted or unsubstituted cyclic or aromatic ring.

In another embodiment, the agonists or antagonists that can be utilized as part of the methods of the present are compounds of structural formula (XI): 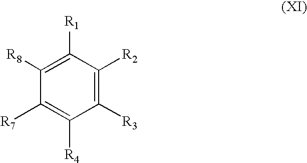
or a pharmaceutically available solvate or hydrate thereof, wherein;

each of R₁, R₂, R₃, R₄, R₅, R₇ or R₈ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(CH₂)ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅—C₁₀)heteroaryl, —(C₅-C₁₀)cycloheteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 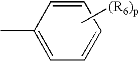
wherein;

each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

R₁ and R₂ can optionally together form a 5-, 6- or 7-membered substituted or unsubstituted cyclic or aromatic ring;

R₂ and R₃ can optionally together form a 5-, 6- or 7-membered substituted or unsubstituted cyclic or aromatic ring;

R₃ and R₄ can optionally together form a 5-, 6- or 7-membered substituted or unsubstituted cyclic or aromatic ring;

R₄ and R₇ can optionally together form a 5-, 6- or 7-membered substituted or unsubstituted cyclic or aromatic ring;

R₇ and R₈ can optionally together form a 5-, 6- or 7-membered substituted or unsubstituted cyclic or aromatic ring; and

R₁ and R₈ can optionally together form a 5-, 6- or 7-membered substituted or unsubstituted cyclic or aromatic ring.

In another embodiment, the agonists or antagonists that can be utilized as part of the methods of the present invention are compounds of structural formula (XII): 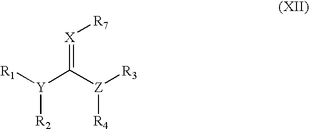
or a pharmaceutically available solvate or hydrate thereof, wherein;

each of R₁, R₂, R₃, R₄, R₅ or R₇ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(CH₂)ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl,

—(C₅-C₁₀)cycloheteroaryl, —(C₃-C₆)cycloheteroalkyl, -naphthyl, —(C₃-C₁₀)heterocycle, —CO₂(CH₂)ₘR₅, —NHC(O)R₅, —NHC(O)OR₅, —NHC(O)NHR₅, —NR₅R₅, —NR₅, —(C₁-C₁₀)alkylNHC(O)(CH₂)ₘR₅, —(C₃-C₁₀)cycloheteroalkyl(R₅)ₘ, —(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, SR₅, —S(O)R₅, —S(O)₂R₅, —S(O)₂NHR₅, or 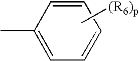
wherein;

each R₅ or R₆ is independently —H, -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

R₃ or R₄ can optionally form a substituted or unsubstituted cyclic, aromatic, heterocyclic, heteroaryl or cycloheteroalkyl ring;

R₁ or R₂ can optionally form a substituted or unsubstituted cyclic, aromatic, heterocyclic, heteroaryl or cycloheteroalkyl ring; and

R₂ or R₄ can optionally form a substituted or unsubstituted cyclic, aromatic, heterocyclic, heteroaryl or cycloheteroalkyl ring.

### 4. BRIEF DESCRIPTION OF THE FIGURES

FIG. 1 illustrates the selectivity of 101 for the Edg-4 receptor;

FIG. 2 illustrates a dose response curve for Edg-4 antagonists 101, 103 and 105;

FIG. 3 illustrates a dose response curve for 101 and LPA in HTC rat hepatoma cells transfected with human Edg-4 receptors;

FIG. 4 illustrates inhibition of LPA induced calcium mobilization by the Edg-4 antagonist 101 in OV202 human ovarian cancer cells;

FIG. 5 illustrates a dose response curve for 101 in CaOV-3 human ovarian cancer cells;

FIG. 6 illustrates the inhibition of VEGF production by 101 in CaOV-3 human ovarian cancer cells;

FIG. 7 illustrates the inhibition of IL-8 production by 101 in CaOV-3 human ovarian cancer cells;

FIG. 8 illustrates the inhibition of LPA-stimulated proliferation by 101 in CaOV-3 human ovarian cancer cells;

FIG. 9 illustrates the inhibition of LPA-stimulated chemotaxis by 103 in CaOV-3 human ovarian cancer cells;

FIG. 10 illustrates the lack of inhibition of SIP-stimulated migration by 103 in human umbilical vein endothelial cells;

FIG. 11 illustrates a dose response inhibition curve of LPA induced calcium mobilization by the Edg-4 antagonists 101, 103, 107 and 113 in HTC rat hepatoma cells transfected with human Edg-4;

FIG. 12 illustrates a dose response inhibition curve of LPA induced calcium mobilization by the Edg-4 antagonists 101, 103, 107 and 113 in HTC rat hepatoma cells transfected with pooled rat Edg-4 clones;

FIG. 13 illustrates a dose response inhibition curve of LPA induced calcium mobilization by the Edg-4 antagonists 101, 103, 107 and 113 in HTC rat hepatoma cells transfected with pooled mouse Edg-4 clones;

FIG. 14 illustrates the efficacy of 101 in suppressing the tumor growth as tested by in vivo Z-chamber study;

FIG. 15 illustrates a dose response curve of calcium mobilization by the Edg-4 agonist 125 on HTC cells transfected with Eag-4 with and without the Edg-4 antagonist 103; and

FIG. 16 illustrates a dose response curve of calcium mobilization by the Edg-4 agonist 125 on CaOV3 cells with and without the Edg-4 antagonist 103.

### 5. DETAILED DESCRIPTION OF THE INVENTION

5.1. Definitions

“Compounds of the invention” refers generally to any modulator of the LPA2 or Edg-4 receptor (e.g. human Edg-4, GenBank Accession Nos. AF23 3092 or AFO1 1466) and includes any Edg-4 receptor modulator encompassed by generic formulae disclosed herein and further includes any species within those formulae whose structure is disclosed herein. The compounds of the invention may be identified either by their chemical structure and/or chemical name. If the chemical structure and chemical name conflict, the chemical structure is determinative of the identity of the compound. The compounds of the invention may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds of the invention may also exist in several tautomeric forms including, but not limited to, the enol form, the keto form and mixtures thereof. Accordingly, the chemical structures depicted herein encompass all possible tautomeric forms of the illustrated compounds. The compounds of the invention also include isotopically labeled compounds where one or more atoms have an atomic mass different from the atomic mass conventionally found in nature. Examples of isotopes that may be incorporated in the compounds of the invention include, but are not limited to, ²H, ³H, ¹³C, ¹⁴C, ¹⁵N, ¹⁸O, ¹⁷O, ³¹P, ³²P, ³⁵S, ¹⁸F and ³⁶Cl. Further, it should be understood that when partial structures of the compounds of the invention are illustrated, brackets indicate the point of attachment of the partial structure to the rest of the compound.

“Composition of the invention” refers to at least one compound of the invention and a pharmaceutically acceptable vehicle, with which the compound is administered to a patient. When administered to a patient, the compounds of the invention are administered in isolated form, which means separated from a synthetic organic reaction mixture.

“Alkyl” refers to a saturated or unsaturated, branched, straight-chain or cyclic monovalent hydrocarbon group derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan- 1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl , but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 20 carbon atoms.

“Alkanyl” refers to a saturated branched, straight-chain or cyclic alkyl group derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” refers to an unsaturated branched, straight-chain or cyclic alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” refers to a radical —C(O)R, where R is hydrogen, alkyl, cycloalkyl, cycloheteroalkyl, aryl, arylalkyl, heteroalkyl, heteroaryl, heteroarylalkyl as defined herein. Representative examples include, but are not limited to formyl, acetyl, cylcohexylcarbonyl, cyclohexylmethylcarbonyl, benzoyl, benzylcarbonyl and the like.

“Acylamino” refers to a radical —NR′C(O)R, where R′ and R are each independently hydrogen, alkyl, cycloalkyl, cycloheteroalkyl, aryl, arylalkyl, heteroalkyl, heteroaryl, heteroarylalkyl, as defined herein. Representative examples include, but are not limited to, formylamino, acetylamino, cylcohexylcarbonylamino, cyclohexylmethyl-carbonylamino, benzoylamino, benzylcarbonylamino and the like.

“Alkylamino” means a radical —NHR where R represents an alkyl or cycloalkyl group as defined herein. Representative examples include, but are not limited to, methylamino, ethylamino, 1-methylethylamino, cyclohexyl amino and the like.

“Alkoxy” refers to a radical —OR where R represents an alkyl or cycloalkyl group as defined herein. Representative examples include, but are not limited to, methoxy, ethoxy, propoxy, butoxy, cyclohexyloxy and the like.

“Alkoxycarbonyl” refers to a radical —C(O)-alkoxy where alkoxy is as defined herein.

“Alkylarylamino” refers to a radical —NRR′ where R represents an alkyl or cycloalkyl group and R′ is an aryl as defined herein

“Alkylsulfonyl” refers to a radical —S(O)₂R where R is an alkyl or cycloalkyl group as defined herein. Representative examples include, but are not limited to methylsulfonyl, ethylsulfonyl, propylsulfonyl, butylsulfonyl and the like.

“Alkylsulfinyl” refers to a radical —S(O)R where R is an alkyl or cycloalkyl group as defined herein. Representative examples include, but are not limited to, methylsulfinyl, ethylsulfinyl, propylsulfinyl, butylsulfinyl and the like.

“Alkylthio” refers to a radical —SR where R is an alkyl or cycloalkyl group as defined herein that may be optionally substituted as defined herein. Representative examples include, but are not limited to methylthio, ethylthio, propylthio, butylthio, and the like.

“Amino” refers to the radical —NH₂.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Preferably, an aryl group comprises from 6 to 20 carbon atoms.

“Arylalkyl” refers to an acyclic alkyl group in which one of the hydrogen atoms bonded to a carbon atom, typically a terminal or sp3 carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₆-C₃₀) arylalkyl, e.g., the alkanyl, alkenyl or alkynyl moiety of the arylalkyl group is (C₁-C₁₀) and the aryl moiety is (C₆-C₂₀).

“Arylalkyloxy” refers to an -O-arylalkyl radical where arylalkyl is as defined herein.

“Arylamino” means a radical —NHR where R represents an aryl group as defined herein.

“Aryloxycarbonyl” refers to a radical —C(O)—O-aryl where aryl is as defined herein.

“Azido” refers to the radical —N₃.

“Carbamoyl” refers to the radical —C(O)N(R)₂ where each R group is independently hydrogen, alkyl, cycloalkyl or aryl as defined herein, which may be optionally substituted as defined herein.

“Carboxy” means the radical —C(O)OH.

“Cyanato” means the radical —OCN.

“Cyano” means the radical —CN.

“Cycloalkyl” refers to a saturated or unsaturated cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In a preferred embodiment, the cycloalkyl group is (C₃-C₁₀) cycloalkyl, more preferably (C₃-C₆) cycloalkyl.

“Cycloheteroalkyl” refers to a saturated or unsaturated cyclic alkyl group in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from dioxanes, dioxolanes, epoxides, imidazolidine, morpholine, piperazine, piperidine, pyrazolidine, pyrrolidine, quinuclidine, tetrahydrofuran, tetrahydropyran and the like.

“Cycloheteroalkyloxycarbonyl” refers to a radical —C(O)—OR where R is cycloheteroalkyl is as defined herein.

“Dialkylamino” means a radical —NRR′ where R and R′ independently represent an alkyl or cycloalkyl group as defined herein. Representative examples include, but are not limited to dimethylamino, methylethylamino, di-(1-methylethyl)amino, (cyclohexyl)(methyl)amino, (cyclohexyl)(ethyl)amino, (cyclohexyl)(propyl)amino, and the like.

“Halo” means fluoro, chloro, bromo, or iodo.

“Haloalkyl” means an alkyl radical substituted by one or more halo atoms wherein alkyl and halo is as defined herein.

“Heteroalkyloxy” means an, —O-heteroalkyl group where heteroalkyl is as defined herein.

“Heteroalkyl, Heteroalkanyl, Heteroalkenyl, Heteroalkvnyl” refer to alkyl, alkanyl, alkenyl and alkynyl groups, respectively, in which one or more of the carbon atoms (and any associated hydrogen atoms) are each independently replaced with the same or different heteroatomic groups. Typical heteroatomic groups include, but are not limited to, —O—, —S—, —O—O—, —S—S—, —O—S—, —NR′—, ═N—N═, —NN—, —N═N—NR—, —PH—,—P(O)₂—, —O—P(O)₂—, —S(O)—, —S(O)₂—, —SnH₂— and the like, wherein R′ is hydrogen, alkyl, substituted alkyl, cycloallcyl, substituted cycloalkyl, aryl or substituted aryl.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered heteroaryl being particularly preferred. Preferred heteroaryl groups are those derived from thiophene, pyrrole, benzothiophene, benzofuran, indole, pyridine, quinoline, imidazole, oxazole and pyrazine.

“Heteroaryloxy” refers to an —O-heteroarylalkyl radical where heteroarylalkyl is as defined herein.

“Heteroaryloxycarbonyl” refers to a radical —C(O)—OR where R is heteroaryl as defined herein.

“Heteroarylalkyl” refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In preferred embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20 membered heteroaryl.

“Hydroxy” refers to the radical —OH.

“Leaving group” has the meaning conventionally associated with it in synthetic organic chemistry, i.e., an atom or a group capable of being displaced by a nucleophile and includes halo (such as chloro, bromo, and iodo), alkoxycarbonyl (e.g., acetoxy), aryloxycarbonyl, mesyloxy, tosyloxy, trifluoromethanesulfonyloxy, aryloxy (e.g, 2,4-dinitrophenoxy), methoxy, N,O-dimethylhydroxylamino, and the like.

“Nitro” refers to the radical —NO₂.

“Oxo” refers to the divalent radical ═O.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chiorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, Nmethylglucamine and the like.

“Pharmaceutically acceptable vehicle” refers to a diluent, adjuvant, excipient or carrier with which a compound of the invention is administered.

“Patient” includes humans. The terms “human” and “patient” are used interchangeably herein.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Prodrug” refers to a pharmacologically inactive derivative of a drug molecule that requires a transformation within the body to release the active drug. Typically, prodrugs are designed to overcome pharmaceutical and/or pharmacokinetically based problems associated with the parent drug molecule that would otherwise limit the clinical usefulness of the drug.

“Promoiety” refers to a form of protecting group that when used to mask a functional group within a drug molecule converts the drug into a prodrug. Typically, the promoiety will be attached to the drug via bond(s) that are cleaved by enzymatic or non-enzymatic means in vivo. Ideally, the promoiety is rapidly cleared from the body upon cleavage from the prodrug.

“Protecting group” refers to a grouping of atoms that when attached to a reactive group in a molecule masks, reduces or prevents that reactivity. Examples of protecting groups can be found in Green et al, “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilylethanesulfonyl (“SES”), trityl and substituted trityl groups, allyloxycarbonyl, 9-fluorenylmethyloxycarbonyl (“FMOC”), nitroveratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Substituted” refers to a group in which one or more hydrogen atoms are each independently replaced with the same or different substituent(s). Typical substituents include, but are not limited to, —X, —R₁₄, —OS, ═O, —OR₁₄, —SR₁₄, S^{—}, ═S —NR₁₄, R₁₅, ═NR₁₄,—CX₃, —CF₃, —CN, —OCN, —SCN, —NO, —NO₂, ═N₂, —N₃, —S(O)₂O—, —S(O)₂OH, —S(O)₂R₁₄, —OS(O₂)O^{—}, —OS(O)₂R₁₄, —P(O)(O)₂, —P(O)(OR₁₄)(O), —OP(O)(OR₁₄)(OR₁₅), —C(O)R₁₄, —C(S)R₁₄, —C(O)OR₁₄, —C(O)NR₁₄R₁₅, —C(O)O, —C(S)OR₁₄, —NR₁₆C(O)NR₁₄R₁₅, —NR₁₆C(S)NR₁₄R₁₅, ^{—}NR₁₇C(NR₁₆)NR₁₄R₁₅ and —C(NR₁₆)NR₁₄R₁₅, where each X is independently a halogen; each R₁₄, R₁₅, R₁₆ and R₁₇ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR₁₅R₁₉, —C(O)R₁₅ or —S(O)₂R₁₅ or optionally R₁₅ and R₁₉ together with the atom to which they are both attached form a cycloheteroalkyl or substituted cycloheteroalkyl ring; and R₁₅ and R₁₉ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroallcyl, substituted heteroalkyl, heteroaryl, substituted heteroaryl, heteroarylalkyl or substituted heteroarylalkyl.

“Sulfonylamino” refers to a radical —NR′S(O₂)R, where R′ and R are each independently hydrogen, alkyl, cycloalkyl, cycloheteroalkyl, aryl, arylalkyl, heteroalkyl, heteroaryl, heteroarylalkyl, as defined herein.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

“Thio” refers to the radical —SH.

“Thiocyanato” refers to the radical —SCN.

“Thiono” refers to the divalent radical ═S.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

5.2. The Use of the Compounds of the Invention

The present invention provides a method of modulating an LPA2 or Edg-4 receptor (e.g., human Edg-4, GenBank Accession Nos. AF233092 or AFOI 1466) mediated biological activity. A cell expressing the Edg-4 receptor is contacted with an amount of an Edg-4 receptor agonist or antagonist sufficient to modulate the Edg-4 receptor mediated biological activity.

Those of skill in the art will appreciate that Edg-4 is a G protein coupled receptor (“GPCR”). The Edg-4 (LPA2) receptor is encoded by an endothelial differentiation gene and along with related receptors, Edg-2 (LPA1) and Edg-7 (LPA3), binds lysophosphatidic acid (“LPA”). Preferably, the Edg-4 receptor is a human receptor.

The Edg-4 receptor may be expressed by recombinant DNA methods well known to those of skill in the art. Particularly useful cell types for expressing and assaying Edg-4 include, but are not limited to, HTC4 (rat hepatoma cells), RH7777 (rat hepatoma cells), HepG2 (human hepatoma cells), CHO (Chinese hamster ovary cells) and HEK-293 (human embryonic kidney cells). Particularly useful vectors for expressing G-protein receptors include, but are not limited to, pLXSN and pCMV (Clontech Labs, Palo Alto, Calif.; Invitrogen Corporation, Carlsbad, Calif.).

DNA encoding Edg-4 is well known (e.g, human Edg-4, GenBank Accession Nos. AF233092 or AF011466) and can be transfected into human or mammalian cells according to methods known to those of skill in the art. For example, DNA encoding human Edg-4 can be co-transfected with a standard packaging vector, such as those described above, which provides an ecotropic envelope for viral replication, into a packaging cell line such as GP-293 (C₁₀ntech Labs, Palo Alto, Calif.).

Alternatively, DNA encoding Edg-4 can be transfected into the EcoPack-293 cell line which has, in addition to gag and pol, the env gene to produce an ecotropic envelope. Both methods (i.e., co-transfection with a packaging vector or use of EcoPack-293) enable the production of an ecotropic envelope for viral packaging, and can thus advantageously be used to transfect rat and mouse cells. For use in human and other mammalian cells, AmphoPack-293 cell line can be used (Clontech Labs, Palo Alto, Calif.).

In addition, a number of natural cell lines naturally express Edg-4 receptors.

These include, but are not limited to, CaOV-3 human ovarian cancer cells, MDA-MB453 and MDA-MB-231 breast cancer cells, HT-1080 human fibrosarcoma, HUVEC cells and OV202 human ovarian cancer cells (ATCC, Manassas, Va.; Vec Technologies Inc. (Rensselaer, N.Y.); Dr. Edward Goetzl, University of California, San Francisco, San Francisco, Calif.).

Those of skill in the art will appreciate that cells which express the Edg-4 receptor may grown in vitro or may be part of a complex organism such as, for example, a mammal. It is contemplated that the methods of the current invention will be applicable to modulation of Edg-4 receptor activity, regardless of the local environment. In one preferred embodiment, cells that express the Edg-4 receptor are grown in vitro (i.e., are cultured). In another preferred embodiment, cells that express the Edg-4 receptor are in vivo (i.e., are part of a complex organism).

The cells, in which the method of the invention may be practiced include, but are not limited to, hepatoma cells, ovarian cells, epithelial cells, fibroblast cells, neuronal cells, cardiac myocytes, endothelial cells, carcinoma cells, pheochromocytoma cells, myoblast cells, platelet cells and fibrosarcoma cells. More specifically, the cells in which the invention may be practiced include, but are not limited to, 0V202 human ovarian cells, HTC rat hepatoma cells, CAOV-3 human ovarian cancer cells, MDA-MB-453 breast cancer cells, MDA-MB-231 breast cancer cells, HUVEC, A431 human epitheloid carcinoma cells and HT-1080 human fibrosarcoma cells.

In another aspect, an Edg-4 receptor mediated biological activity is modulated in a subject or in an animal model. A therapeutically effective amount of a modulator of the Edg-4 receptor is administered to the subject or an animal. Preferably, the subject or animal is in need of such treatment.

The biological activity mediated by the Edg-4 receptor may include, for example, calcium mobilization, VEGF synthesis, IL-8 synthesis, platelet activation, cell migration, phosphoinositide hydrolysis, inhibition of cAMP formation or actin polymerization. Preferably, the biological activity mediated by the Edg-4 receptor includes, but is not limited to, apoptosis, angiogenesis, inhibition of wound healing, inflammation, cancer invasiveness or atherogenesis. Most preferably, the biological activity mediated by the Edg-4 receptor is cell proliferation, which may lead to ovarian cancer, peritoneal cancer, endometrial cancer, cervical cancer, breast cancer, colon cancer or prostrate cancer. In one embodiment, cell proliferation is stimulated by LPA.

In another embodiment, the biological activity mediated by the Edg-4 receptor may include increasing fatty acids levels (e.g., free fatty acids and lysophosphatidylcholine) which may lead to acute lung diseases, such as adult respiratory distress syndrome (“ARDS”) and acute inflammatory exacerbation of chronic lung diseases like asthma.

In yet another embodiment, compounds that block Edg-4 can be potentially effective immunosuppressive agents because activated T cells have Edg-4 receptors (Zheng et al., 2000, FASEB J 14:2387-2389). Edg-4 antagonists may be useful in a variety of autoimmune and related immune disorders, including, but not limited to, systemic lupus erythematosus (SLE), rheumatoid arthritis, non-glomerular nephrosis, psoriasis, chronic active hepatitis, ulcerative colitis, Crohn's disease, Behoet's disease, chronic glomerulonephritis, chronic thrombocytopenic purpura, and autoimmune hemolytic anemia. Additionally, Edg-4 antagonists can be used in organ transplantation.

In one embodiment, the modulator exhibits selectivity for the Edg-4 receptor. For example, the modulator exhibits at least about 5 to about 200 fold inhibitory selectivity for Edg-4 relative to other Edg receptors. Inhibitory selectivity, can be measured by assays such as a calcium mobilization assay or a migration and/or invasion assay or a proliferation assay, for example, as described in Section 6.26 (Example 26), 6.28 (Example 28) and 6.29 (Example 29) respectively. In a preferred embodiment, inhibitory selectivity can be measured by a calcium mobilization assay. Other assays suitable for determining inhibitory selectivity would be known to one of skill in the art.

In some embodiments, the modulator exhibits at least about 200 fold inhibitory selectivity for Edg-4 relative to other non-Edg receptors, including, but not limited to, other GPCRs, ion channels, growth factor receptors and the like.

In other embodiments, the modulator exhibits at least about 63 fold inhibitory selectivity for Edg-4 relative to other Edg receptors.

In another embodiment, the modulator exhibits at least about 30 fold inhibitory selectivity for Edg-4 relative to other Edg receptors.

In still another embodiment, the modulator exhibits at least about 10 fold inhibitory selectivity for Edg-4 relative to other Edg receptors.

In one embodiment, the modulator exhibits at least about 5 fold inhibitory selectivity for Edg-4 relative to other Edg receptors.

In another embodiment, the modulator exhibits at least about 200 fold inhibitory selectivity for Edg-4 relative to Edg-2 and Edg-7 receptors.

In yet another embodiment, the modulator exhibits at least about 63 fold inhibitory selectivity for Edg-4 relative to Edg-2 and Edg-7 receptors.

In another embodiment, the modulator exhibits at least about 30 fold inhibitory selectivity for Edg-4 relative to Edg-2 and Edg-7 receptors.

In still another embodiment, the modulator exhibits at least about 10 fold inhibitory selectivity for Edg-4 relative to Edg-2 and Edg-7 receptors.

In still another embodiment, the modulator exhibits at least about 5 fold inhibitory selectivity for Edg-4 relative to Edg-2 and Edg-7 receptors.

In a preferred embodiment, the modulator of cell proliferation exhibits at least about 200 fold inhibitory selectivity for Edg-4 relative to other Edg receptors.

In another embodiment, the modulator of cell proliferation exhibits at least about 10 fold inhibitory selectivity for Edg-4 relative to other Edg receptors.

In still another embodiment, the modulator of cell proliferation exhibits at least about 10 fold inhibitory selectivity for Edg-4 relative to Edg-2 and Edg-7 receptors.

In still another embodiment, the modulator of cell proliferation exhibits at least about 200 fold inhibitory selectivity for Edg-4 relative to Edg-2 and Edg-7 receptors.

In another embodiment, the modulator exhibits activating selectivity for the Edg-4 receptor. For example, the modulator exhibits at least about 5 to about 200 fold activating selectivity for Edg-4 relative to other Edg receptors. Activating selectivity, can be measured by assays such as a calcium mobilization assay or a migration and/or invasion assay or a proliferation assay, for example, as described in Section 6.26 (Example 26), 6.28 (Example 28) and 6.29 (Example 29) respectively. In a preferred embodiment, activating selectivity can be measured by a calcium mobilization assay. Other assays suitable for determining activating selectivity would be known to one of skill in the art.

In one embodiment, the modulator exhibits at least about 200 fold activating selectivity for Edg-4 relative to other non-Edg receptors, including, but not limited to, other GPCRs, ion channels, growth factor receptors and the like.

In another embodiment, the modulator exhibits at least about 63 fold activating selectivity for Edg-4 relative to other Edg receptors.

In another embodiment, the modulator exhibits at least about 30 fold activating selectivity for Edg-4 relative to other Edg receptors.

In another embodiment, the modulator exhibits at least about 10 fold activating selectivity for Edg-4 relative to other Edg receptors.

In one embodiment, the agonist modulator exhibits at least about 5 fold activating selectivity for Edg-4 relative to other Edg receptors.

In still another embodiment, the modulator exhibits at least about 200 fold activating selectivity for Edg-4 relative to Edg-2 and Edg-7 receptors.

In yet another embodiment, the modulator exhibits at least about 63 fold activating selectivity for Edg-4 relative to Edg-2 and Edg-7 receptors.

In another embodiment, the modulator exhibits at least about 30 fold activating selectivity for Edg-4 relative to Edg-2 and Edg-7 receptors.

In still another embodiment, the modulator exhibits at least about 10 fold activating selectivity for Edg-4 relative to Edg-2 and Edg-7 receptors.

In still another embodiment, the modulator exhibits at least about 5 fold activating selectivity for Edg-4 relative to Edg-2 and Edg-7 receptors.

In a preferred embodiment, of cell proliferation exhibits at least about 200 fold activating selectivity for Edg-4 relative to other Edg receptors.

In another embodiment, the modulator of cell proliferation exhibits at least about 10 fold activating selectivity for Edg-4 relative to other Edg receptors.

In still another embodiment, the modulator of cell proliferation exhibits at least about 10 fold activating selectivity for Edg-4 relative to Edg-2 and Edg-7 receptors.

In still another embodiment, the modulator of cell proliferation exhibits at least about 200 fold activating selectivity for Edg-4 relative to Edg-2 and Edg-7 receptors.

In one embodiment, the Edg-4 modulator is not a lipid. In another embodiment, the modulator of Edg-4 receptor mediated biological activity does not contain a phosphate group such as a phosphoric acid, a cyclic phosphate ester or a linear phosphate ester. In another embodiment, the modulator of the Edg-4 receptor is not a phospholipid. The term “phospholipid” includes all phosphate (both phosphate esters and phosphoric acids) containing glycerol derivatives with an alkyl chain of greater 10 carbon atoms or greater, any N-acyl ethanolamide phosphate derivative (both phosphate esters and phosphoric acids), LPA, SIP or any of their analogues (both phosphate esters and phosphoric acids) (see, e.g., Bandoh, et al, 2000, FEBS Lett. 428, 759; Bittman et al., 1996, J. Lipid Research 391; Lilliom et al, 1996, Molecular Pharmacology 616, Hooks et al, 1998, Molecular Pharmacology 188; Fischer et al, 1998, Molecular Pharmacology 979; Heise et al, 2001, Molecular Pharmacology 1173; Hopper et al., 1999, J. Med. Chem. 42 (6):963-970; Tigyi et al, 2001, Molecular Pharmacology 1161).

In another embodiment, the modulator is also not a compound of structural formula: 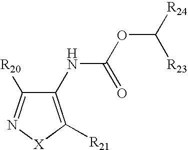
or a pharmaceutically available salt thereof, wherein:

X is O or S;

R₂₀ is alkyl, substituted alkyl, aryl, substituted aryl or halo;

R₂₁ is alkyl, substituted alkyl, aryl, substituted aryl, heteroaryl or substituted heteroaryl;

R₂₃ is hydrogen, alkyl or substituted alkyl;

R₂₄ is aryl, substituted aryl, heteroaryl or substituted heteroaryl; or alternatively R₂₃ and R₂₄ form a cycloalkyl ring (International Application No: WO 01/60819).

In another embodiment, the modulator is not any compound of the formula below: 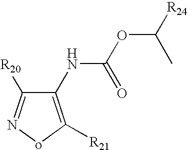
wherein R₂₀, R₂₁, and R₂₄ are as previously defined. In yet another embodiment the modulator is not any compound disclosed in International Application No: WO 01/60819.

The Edg-4 modulator may be a biomolecule such as a nucleic acid, protein, (i.e., an enzyme or an antibody) or oligosaccharide or any combination thereof. Alternatively, the Edg-4 modulator may be oligomers or monomers of the above biomolecules such as amino acids, peptides, monosaccharides, disaccharides, nucleic acid monomers, dimers, etc., or any combination thereof. The Edg-4 modulator may also be a synthetic polymer or any combination of synthetic polymer with biomolecules including monomers or oligomers of biomolecules.

The Edg-4 modulator may also be an organic molecule of molecular weight less than 750 daltons. In one embodiment, the molecular weight is about 200 to about 1000 daltons. In another embodiment, the molecular weight is about 200 to about 750 daltons. In yet another embodiment, the molecular weight is about 200 to about 500 daltons. Preferably, the molecular weight is about 300 to about 500 daltons.

Without wishing to be bound by any particular theory or understanding, the modulator may, for example, facilitate inhibition of the Edg-4 receptor through direct binding to the LPA binding site of the receptor, binding at some other site of the Edg-4 receptor, interference with Edg-4 or LPA biosynthesis, covalent modification of either LPA or the Edg-4 receptor, or may otherwise interfere with Edg-4 mediated signal transduction.

In one embodiment, the agonist or antagonist binds to the Edg-4 receptor with a binding constant between about 10 μM and about 1 μM. In another embodiment, the modulator binds to the Edg-4 receptor with a binding constant between about 10 μM and about 1 nM. In another embodiment, the modulator binds to the Edg-4 receptor with a binding constant between about 1 μM and about 1 nM. In another embodiment, the modulator binds to the Edg-4 receptor with a binding constant between about 100 nM and about 1 nM. In another embodiment, the modulator binds to the Edg-4 receptor with a binding constant between about 10 nM and about 1 nM. Preferably, the modulator binds to the Edg-4 receptor with a binding constant better (i.e., less) than about 10 nM.

In a specific embodiment, the modulator is a compound of structural formula (I): 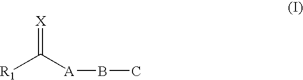
or a pharmaceutically available solvate or hydrate thereof, wherein:

R₁ is hydrogen, alkyl, substituted alkyl, acylamino, substituted acylamino, alkylamino, substituted alkylamino, alkylthio, substituted alkylthio, alkoxy, substituted alkoxy, alkylarylamino, substituted alkylarylamino, amino, arylalkyloxy, substituted arylalkyloxy, aryl, substituted aryl, arylamino, substituted arylamino, arylalkyl, substituted arylalkyl, dialkylamino, substituted dialkylamino, cycloalkyl, substituted cycloalkyl, cycloheteroalkyl, substituted cycloheteroalkyl, heteroaryloxy, substituted heteroaryloxy, heteroaryl, substituted heteroaryl, heteroalkyl, substituted heteroalkyl sulfonylamino or substituted sulfonylamino;

X═O or S;

A is NR₂, O or 5;

R₂ is hydrogen, alkyl or substituted alkyl; and

B and C are independently alkyl, substituted alkyl, aryl, substituted aryl, heteroaryl, substituted heteroaryl, cycloalkyl, substituted cycloalkyl, cycloheteroalkyl or substituted cycloheteroalkyl.

Preferably, R₁ is alkyl, substituted alkyl, substituted aryl, substituted aryl, arylalkyloxy or substituted sulfonylamino. More preferably, R₁ is substituted alkyl. Even more preferably, R₁ is substituted haloalkyl. Most preferably, R₁ is substituted trifluoroalkyl (preferably, trifluoroalkanyl).

In a preferred embodiment, R₁ has the structural formula (II):

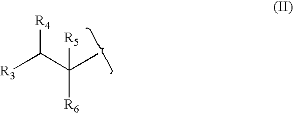

wherein:

R₃ is haloalkyl or substituted haloalkyl;

R₄ is oxo or thiono; and

R₅ and R₆ are independently hydrogen, halo, alkyl or substituted alkyl.

Preferably, R₃ is fluoroalkyl, R₄ is oxo and R₅ and R₆ are independently hydrogen, halo or alkyl. More preferably, R₃ is trifluoromethyl, R₄ is oxo and R₅ and R₆ are independently hydrogen, chloro or methyl.

In one preferred embodiment, R₅ and R6 are hydrogen. In another preferred embodiment, R₅ is hydrogen and R₆ is chloro or methyl.

Preferably in any of the above embodiment, X is O, A is NR₂ and R₂ is hydrogen. In another preferable version of the above embodiments, B and C are alkyl, substituted alkyl, independently, aryl, substituted aryl, heteroaryl or substituted heteroaryl. More preferably, B and C are independently indolo, substituted indolo, imidazolo, substituted, imidazolo, pyrazolo, substituted pyrazolo, phenyl or substituted phenyl. Even more preferably, B is heteroaryl or substituted heteroaryl and C is aryl or substituted aryl. Most preferably, B is pyrazolo or substituted pyrazolo and C is phenyl or substituted phenyl.

In a more specific embodiment, the modulator is a compound of structural formula (III): 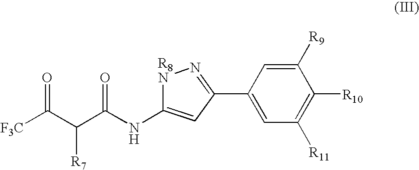
wherein:

R₇ is hydrogen, alkyl, substituted alkyl or halo;

R₈ is hydrogen, carbamoyl or substituted carbamoyl; and

R₉, R₁₀ and R₁₁ are independently hydrogen, alkoxy, substituted alkoxy, halo or optionally, R₉ and R₁₀ together with the carbons to which they are attached form a [1,3] dioxolane ring.

Preferred modulators include compounds of the structural formula shown below:

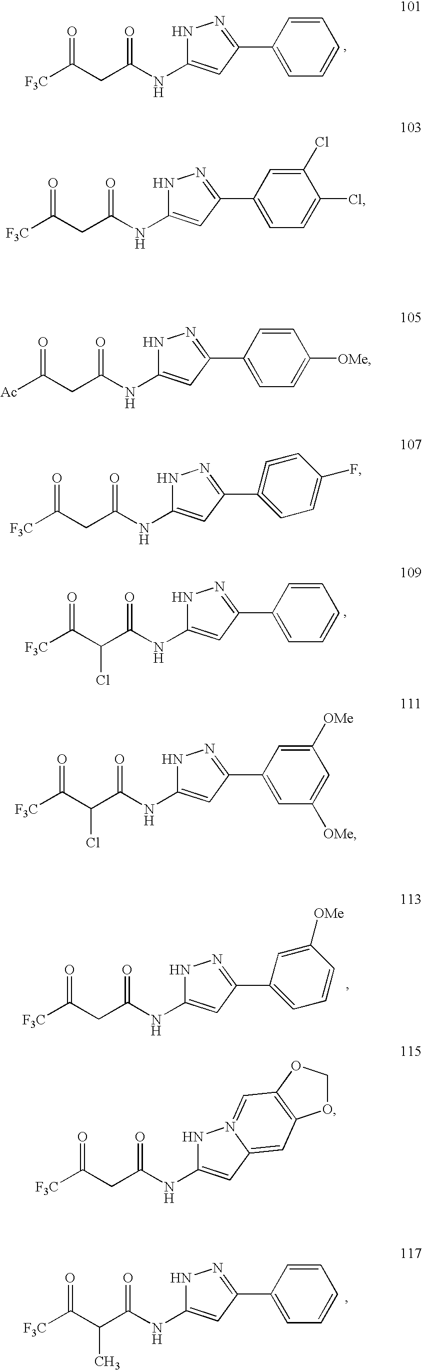

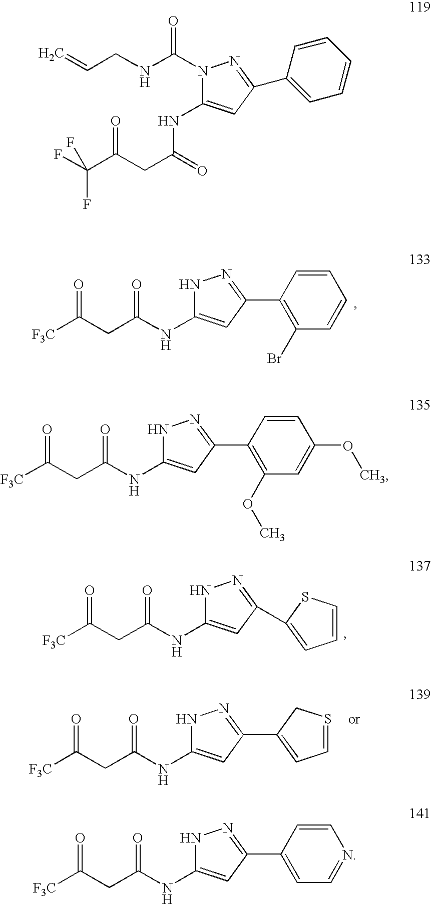

In another embodiment, the agonists or antagonists that can be utilized as part of the methods of the present invention are compounds of structural formula (IV): 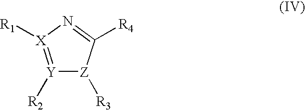
or a pharmaceutically available solvate or hydrate thereof, wherein;

each of R₁, R₂, R₃, R₄ or R₅ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(CH₂)ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, —(C₅-C₁₀)cycloheteroaryl, —(C₃-C₆)cycloheteroalkyl, -naphthyl, —(C₃-C₁₀)heterocycle, —CO₂(CH₂)ₘR₅, —NHC(O)R₅, —NHC(O)OR₅, —NHC(O)NHR₅, —NR₅R₅, ═NR₅, —(C₁-C₁₀)alkylNHC(O)(CH₂)ₘR₅, —(C₃-C₁₀)cycloheteroalkyl(R₅)ₘ, —(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 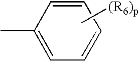
wherein;

each R₅ and R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

X and Y are each independently C or N; and

Z is O, S, C or N, wherein if Z is O or S, then R₃ is an electron pair;

R₁ and R₂ can optionally together form a 5-, 6-, or 7-membered substituted or unsubstituted cyclic or aromatic ring;

R₂ and R₃ can optionally together form a 5-, 6-, or 7-membered substituted or unsubstituted cyclic or aromatic ring; and

R₃ and R₄ can optionally together form a 5-, 6-, or 7-membered substituted or unsubstituted cyclic or aromatic ring.

Some illustrative examples of the modulators of this embodiment include:

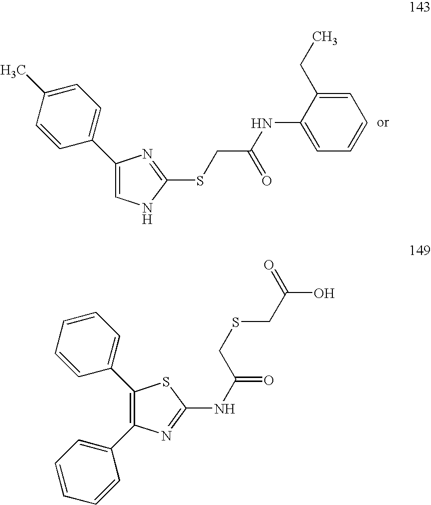

Another embodiment of the present invention is directed to compounds of structural formula (V), which can be utilized for the purpose of this invention: 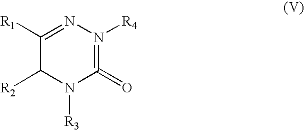
or a pharmaceutically available solvate or hydrate thereof, wherein;

each of R₁, R₂, R₃, R₄ or R₅ is independently —H, -halo, —NO₂, —CN, —OH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHRS, or 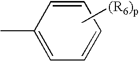
wherein;

each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)(C₁-C₁₀)alkyl, —OC(O)O(C₁-C₁₀)alkyl, or —SO₂NH₂;

m is independently an integer ranging from 0 to 8;

p is independently an integer ranging from 0 to 5; and

R₁ and R₂ or R₂ and R₃ can optionally together form a 5-, 6-, or 7-membered substituted or unsubstituted cyclic or aromatic ring.

In a specific embodiment, R₁ and R₂ are independently aryl, substituted aryl, heteroaryl or substituted heteroaryl. In a more specific embodiment, R₂ is indole, and R₃ and R₄ are hydrogen.

Illustrative examples of the modulators of this embodiment include: 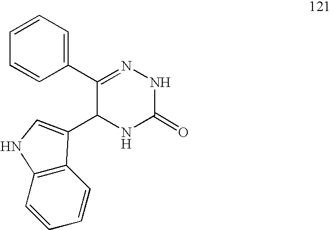
and its (+) and (−) enantiomers.

In another embodiment, the modulator is a compound of structural formula (VI): 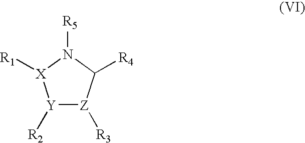
or a pharmaceutically available solvate or hydrate thereof, wherein;

each of R₁, R₂, R₃, R₄ or R₅ is independently —H, -halo, —NO₂, —CN, —OH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -naphthyl, —(C₃-C₁₀)heterocycle, —CO₂(CH₂)ₘR₅, —NHC(O)R₅, —NHC(O)OR₅, —NHC(O)NHR₅, —OC(O)(CH₂)ₘCHR₅R₅, —CO₂(CH₂)ₘCHR₅R₅,—OC(O)OR₅, —SR₅, —S(O)R₅, —S(O)₂R₅, —S(O)₂NHR₅, or 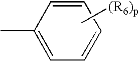
wherein;

R₆ is independently -halo, —NO₂, —CN, —OH, —CO₂H, —N(C₁-C₁₀)alkyl(C₁-C₁₀)alkyl, —O(C₁C₁₀)alkyl, —C(O)(C₁-C₁₀)alkyl, —C(O)NH(CH₂)ₘ(C —C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, CO₂(CH₂)ₘH, —NHC(O)(C₁-C₁₀)alkyl, —NHC(O)NH(C₁-C₁₀)alkyl, —OC(O)(C₁-C₁₀)alkyl, —OC(O)O(C₁-C₁₀)alkyl, or —SO₂NH₂;

m is independently an integer ranging from 0 to 8;

p is independently an integer ranging from 0 to 5;

X, Y and Z are independently O, S, C or N, wherein if X, Y or Z is O or S, R₁ is an electron pair;

R₁ and R₂ can optionally together form a 5-, 6-, or 7-membered substituted or unsubstituted cyclic or aromatic ring;

R₃ and R₄ can optionally together form a 5-, 6- or 7-membered substituted or unsubstituted cyclic or aromatic ring;

R₁ and R₅ can optionally together form a 5-, 6- or 7-membered substituted or unsubstituted cyclic or aromatic ring; and

R₄ and R₅ can optionally together form a 5-, 6- or 7-membered substituted or unsubstituted cyclic or aromatic ring.

In a specific embodiment, R₁ and R₂ together form a 5-, 6- or 7-membered substituted or unsubstituted cyclic or aromatic ring. In a more specific embodiment, R₁ and R₂ together, and R₃ and R₄ together form a 5-, 6- or 7-membered substituted or unsubstituted cyclic or aromatic ring. In an even more specific embodiment, R₁ and R₂ together, and R₃ and R₄ together form a 6-membered substituted or unsubstituted cyclic or aromatic ring. Even more specifically, R₁ and R₂, and R₃ and R₄ form a 6-membered substituted aromatic or cyclic ring.

Illustrative modulators of the invention include, but are not limited to, the following compound:

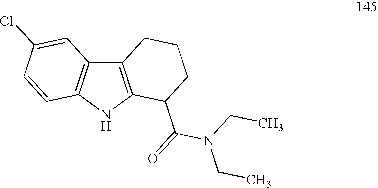

In a specific embodiment, the modulator is a compound of structural formula (VII): 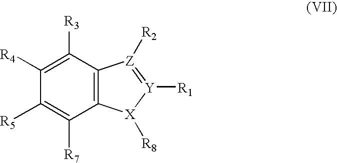
or a pharmaceutically available solvate or hydrate thereof, wherein;

each of R₁, R₂, R₃, R₄, R₅, R₇ or R₈ is independently —H, -halo, —NO₂, —CN, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 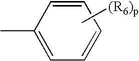
wherein;

each R₅ or R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

X is O, S, C or N, wherein if X is O or S, R₁ is an electron pair; and

Y and Z are independently N or C, wherein if Y or Z is N, R₁ and R₂ are each an electron pair.

Illustrative modulators of the invention include:

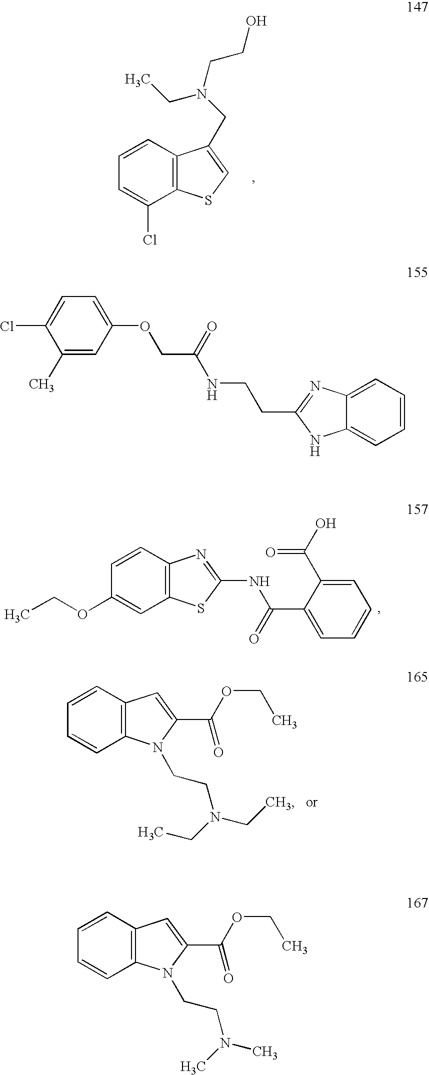

In another specific embodiment, the modulator is a compound of structural formula (VIII): 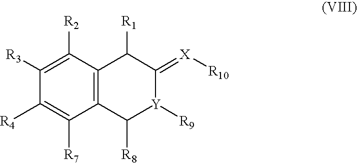
or a pharmaceutically available solvate or hydrate thereof, wherein;

each of R₁, R₂, R₃, R₄, R₅, R₇, R₈, R₉ or R₁₀ is independently —H, -halo, —NO₂, —CN, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 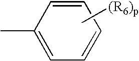
wherein;

each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5; and

X and Y are independently O, S or N, wherein if X or Y is O or S, R₉ and R₁₀ are an electron pair.

In another embodiment, R₇ is a substituted or unsubstituted aryl. An illustrative example of these Egd-4 modulators includes:

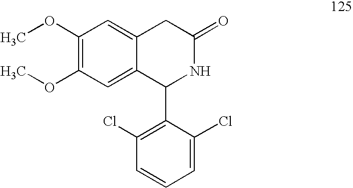

In another embodiment, the modulator is a compound of structural formula (IX) 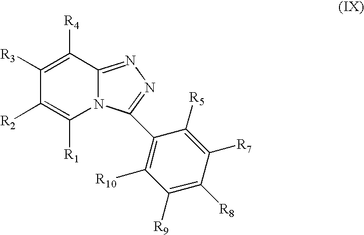
or a pharmaceutically available solvate or hydrate thereof, wherein;

each of R₁, R₂, R₃, R₄, R₅, R₇, R₈, R₉ or R₁₀ is independently —H, -halo, —NO₂, —CN, —C(R₅)₃, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂—C₁₀)alkynyl, —(C₃-C₁₀)cycloalkyl, —(C₈-C₁₄)bicycloalkyl, —(C₅-C₁₀)cycloalkenyl, (C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNk₅R₅, OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 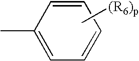
wherein;

each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8; and

p is independently an integer ranging from 0 to 5.

In a specific embodiment, R₂ is a substituted alkyl, and one or more of R₅, R₇, R₈, R₉ and R₁₀ are halos. In a more specific embodiment, R₂ is a halo-substituted alkyl. In an even more specific embodiment, R₂ is CF₃. Specific examples of the modulators include:

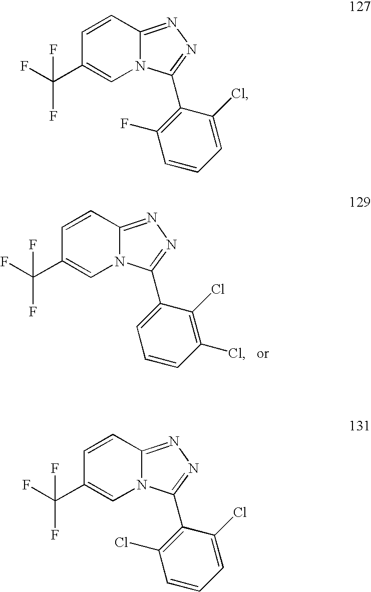

In another specific embodiment, the modulator is a compound of structural formula (X): 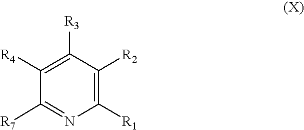
or a pharmaceutically available solvate or hydrate thereof, wherein;

each of R₁, R₂, R₃, R₄, R₅ or R₇ is independently —H, -halo, —NO₂, —CN, —C(R₅)₃, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —CO₂H, —(C₁-C₁₀)alkylC(O)NH(CH₂)ₘR₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 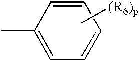
wherein;

each R₅ or R₆ is independently —H, -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

R₁ and R₂ can optionally together form a 5-, 6- or 7-membered substituted or unsubstituted cyclic or aromatic ring;

R₂ and R₃ can optionally together form a 5-, 6- or 7-membered substituted or unsubstituted cyclic or aromatic ring;

R₃ and R₄ can optionally together form a 5-, 6- or 7-membered substituted or unsubstituted cyclic or aromatic ring; and

R₄ and R₇ can optionally together form a 5-, 6- or 7-membered substituted or unsubstituted cyclic or aromatic ring.

In a specific embodiment, R₃ and R₇ are substituted or unsubstituted aryls. An illustrative modulator of the invention includes:

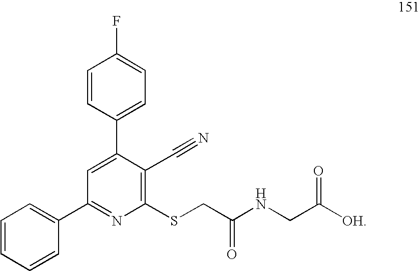

In yet another specific embodiment, the modulator is a compound of structural formula (XI): 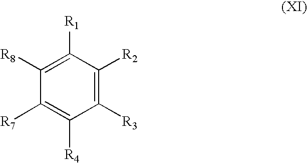
or a pharmaceutically available solvate or hydrate thereof, wherein;

each of R₁, R₂, R₃, R₄, R₅, R₇ or R₈ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(CH₂)ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, —(C₅-C₁₀)cycloheteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 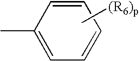
wherein;

each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

R₁ and R₂ can optionally together form a 5-, 6- or 7-membered substituted or unsubstituted cyclic or aromatic ring;

R₂ and R₃ can optionally together form a 5-, 6- or 7-membered substituted or unsubstituted cyclic or aromatic ring;

R₃ and R₄ can optionally together form a 5-, 6- or 7-membered substituted or unsubstituted cyclic or aromatic ring;

R₄ and R₇ can optionally together form a 5-, 6- or 7-membered substituted or unsubstituted cyclic or aromatic ring;

R₇ and R₈ can optionally together form a 5-, 6- or 7-membered substituted or unsubstituted cyclic or aromatic ring; and

R₁ and R₈ can optionally together form a 5-, 6- or 7-membered substituted or unsubstituted cyclic or aromatic ring.

In another embodiment, R₂ and R₃ together form a 5-membered ring. In a more specific embodiment, R₂ and R₃ together, and R₇ and R₈ together form a 5-membered ring. An illustrative example of the modulators of the invention includes:

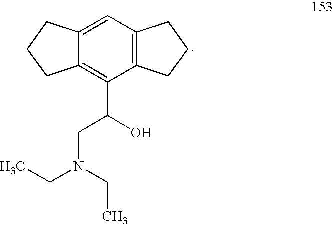

Another illustrative compound of the invention has the following structure:

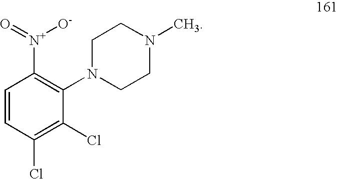

In another specific embodiment, the modulator is a compound of structural formula (XII): 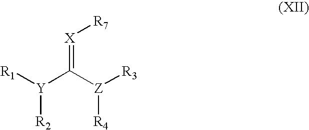
or a pharmaceutically available solvate or hydrate thereof, wherein;

each of R₁, R₂, R₃, R₄, R₅ or R₇ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(CH₂)ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, —(C₅-C₁₀)cycloheteroaryl, —(C₃-C₆)cycloheteroalkyl, -naphthyl, —(C₃-C₁₀)heterocycle, —CO₂(CH₂)ₘR₅, —NHC(O)R₅, —NHC(O)OR₅, —NHC(O)NHR₅, —NR₅R₅, ═NR₅, —(C₁-C₁₀)alkylNHC(O)(CH₂)ₘR₅, —(C₃-C₁₀)cycloheteroalkyl(R₅)ₘ, —(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 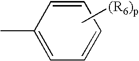
wherein;

each R₅ or R₆ is independently —H, -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;

m is independently an integer ranging from 0 to 8;

p is independently an integer ranging from 0 to 5;

R₃ or R₄ can optionally form a substituted or unsubstituted cyclic, aromatic, heterocyclic, heteroaryl or cycloheteroalkyl ring.

R₁ or R₂ can optionally form a substituted or unsubstituted cyclic, aromatic, heterocyclic, heteroaryl or cycloheteroalkyl ring;

R₂ or R₄ can optionally form a substituted or unsubstituted cyclic, aromatic, heterocyclic, heteroaryl or cycloheteroalkyl ring.

Illustrative examples of the modulators of the invention include:

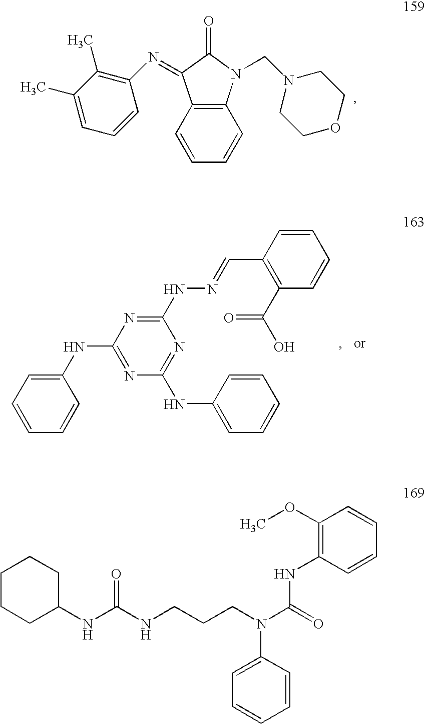

5.3. Synthesis of the Compounds of the Invention

Certain compounds of the invention may be obtained via the synthetic methods illustrated in Schemes 1 and 2. Starting materials useful for preparing compounds of the invention and intermediates thereof are commercially available or can be prepared by well-known synthetic methods. Other methods for synthesis of the compounds described herein are either described in the art or will he readily apparent to the skilled artisan in view of general references well-known in the art (See e.g., Green et al., “Protective Groups in Organic Chemistry”, (Wiley, 2nd ed. 1991); Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the compounds of the invention. Accordingly, the methods presented in Schemes 1 and 2 herein are illustrative rather than comprehensive.

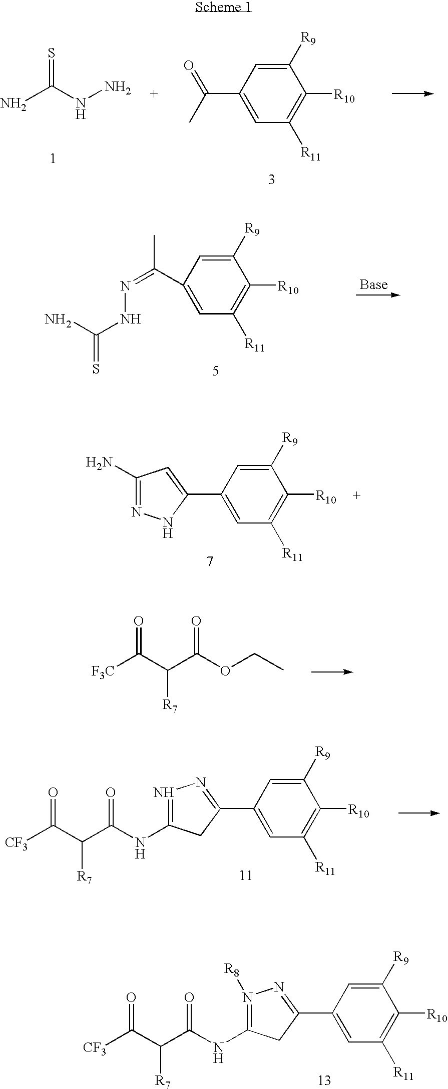

The compounds depicted in Scheme 1 are compounds of structural formula (I). Generally, compounds of structural formula (I) may be made by the route depicted in Scheme 1. Condensation of commercially available thiosemicarbazide 1 with acetophenone 3 in the presence of acid, (e.g., acetic acid) provides thiosemicarbazone 5. In the presence of strong base, (e.g., lithium diisopropylamide) ring formation takes place to form amine 7. Condensation of amine 7 with acetoacetate 9 provides the butyramide 11, which may be alkylated or acylated with an activated urea derivative to provide butyramide 13 (R₈=alkyl, or —C(O)NHR₂₀, where R₂₀ is alkyl).

Those of skill in the art will appreciate that a wide variety of esters other than the acetoacetate 9 depicted may be condensed with amine 7 to provide compounds of the invention. Further the skilled artisan will appreciate that a wide variety of conventional synthetic methods may be used to synthesize compounds of structural Formula (I) other than those depicted above.

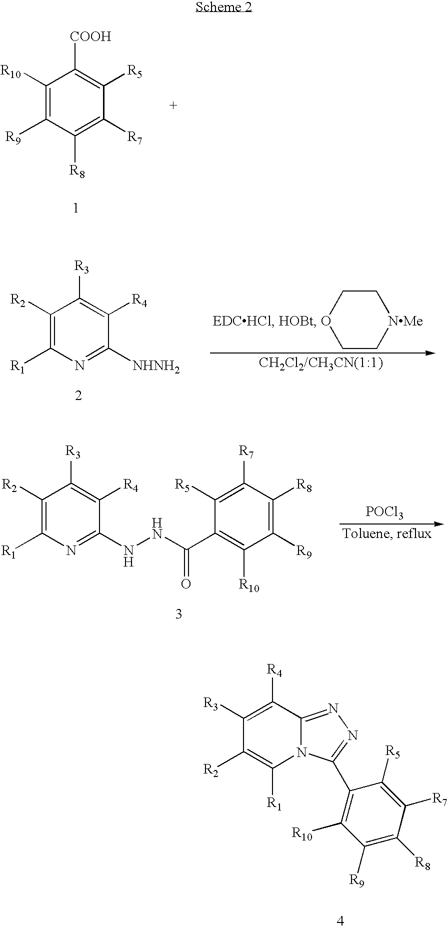

The compounds depicted in Scheme 2 are compounds of structural formula (IX). Generally, compounds of structural formula (IX) may be made by the route depicted in Scheme 2. Unsubstituted or substituted pyridyl hydrazine 2 is reacted with unsubstituted or substituted benzoic acid 1 in the presence of 1-(3-dimethylaminopropyl)-3-ethylcarbodiimide hydrochloride (EDC.HCl), 4-methylmorpholine and 1-hydroxybenzotriazole hydrate (HOBt), in anhydrous 1:1 dichloromethane/acetonitrile. Phosphorous oxychloride is then added to the solution of resulting compound 3 in toluene, and the compound of formula (IX) 4 is obtained.

The skilled artisan will appreciate that a wide variety of conventional synthetic methods may be used to synthesize compounds of structural Formula (IX) other than those depicted above.

Illustrative compounds 145, 147, 149, 151, 153, 155, 157 and 159 are commercially available from Specs (http//:www.specsnet.com); compounds 163, 165 and 167 are available from Chemdiv (http//www.chemdiv.com); compound 161 is available from Tripos (http://www.tripos.com); and compound 169 is available for purchase from Comgenex (http ://www.comgenex.com).

5.4. Therapeutic Uses of the Compounds of the Invention

The compounds and/or compositions of the present invention may be used to treat diseases, including but not limited to, ovarian cancer (Xu et al., 1995, Biochem. J 309 (Pt 3):933-940; Xu et al., 1998, JAMA 280 (8):719-723; Goetzl et al., 1999, Cancer Res. 59 (20):5370-5375), peritoneal cancer, endometrial cancer, cervical cancer, breast cancer, colorectal cancer, uterine cancer, stomach cancer, small intestine cancer, thyroid cancer, lung cancer, kidney cancer, pancreas cancer and prostrate cancer; acute lung diseases, adult respiratory distress syndrome (“ARDS”), acute inflammatory exacerbation of chronic lung diseases such as asthma (Chilton et al., 1996, J Exp Med 183:2235-45; Arbibe et al., 1998, J Clin. Invest 102:1152-60) surface epithelial cell injury, (e.g., transcorneal freezing or cutaneous bums (Liliom et al., 1998, Am. J. Physiol 274 (4 Pt 1): C1065—C1074)), cardiovascular diseases, (e.g., ischemia (Karliner et al., 2001, J. Mol Cell Cardiol. 33 (9):1713-1717) and athescierosis (Siess et al., 1999, Proc. Natl. Acad. Sci. U.S.A 96 (12):6931-6936; Siess et al., 2000, IUBMB.B Life 49 (3):167-171)). In accordance with the invention, a compound and/or composition of the invention is administered to a patient, preferably a human, in need of treatment for a disease which includes but is not limited to, the diseases listed above. Further, in certain embodiments, the compounds and/or compositions of the invention can be administered to a patient, preferably a human, as a preventative measure against diseases or disorders such as those described above. Thus, the compounds and/or compositions of the invention can be administered as a preventative measure to a patient having a predisposition, which includes but is not limited to, the diseases listed above. Accordingly, the compounds and/or compositions of the invention may be used for the prevention of one disease or disorder and concurrently treating another disease (e.g., preventing cancer and treating cardiovascular diseases). It is well within the capability of those of skill in the art to assay and use the compounds and/or compositions of the invention to treat diseases, such as the diseases listed above.

5.5. Therapeutic/Prophylactic Administration

The compounds and/or compositions of the invention may be advantageously used in medicine, including human medicine. As previously described in Section 5.4 above, compounds and compositions of the invention are useful for the treatment or prevention of diseases, which include but are not limited to, cancers, including, but not limited to, ovarian cancer, peritoneal cancer, endometrial cancer, cervical cancer, breast cancer, colorectal cancer, uterine cancer, stomach cancer, small intestine cancer, thyroid cancer, lung cancer, kidney cancer, pancreas cancer, prostrate cancer, acute lung diseases, including, but not limited to, adult respiratory distress syndrome (ARDS) and acute inflammatory exacerbation of chronic lung diseases such as asthma; surface epithelial cell injury, including, but not limited to, transcomeal freezing or cutaneous bums; cardiovascular diseases, including, but not limited to, ischemia and arthesclerosis.

When used to treat or prevent disease or disorders, compounds and/or compositions of the invention may be administered or applied singly, in combination with other agents. The compounds and/or compositions of the invention may also be administered or applied singly, in combination with other pharmaceutically active agents, including other compounds and/or compositions of the invention.

The current invention provides methods of treatment and prophylaxis by administration to a patient of a therapeutically effective amount of a composition or compound of the invention. The patient may be an animal, is more preferably a mammal, and most preferably a human.

The present compounds and/or compositions of the invention, which comprise one or more compounds of the invention, are preferably administered orally. The compounds and/or compositions of the invention may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or composition of the invention. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds and/or compositions of the invention into the bloodstream.

In specific embodiments, it may be desirable to administer one or more compounds and/or composition of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of the diseases listed above.

In certain embodiments, it may be desirable to introduce one or more compounds and/or compositions of the invention into the central nervous system by any suitable route, including intraventricular, intrathecal and epidural injection. Intraventricular injection may be facilitated by an intraventricular catheter, for example, attached to a reservoir, such as an Ommaya reservoir.

A compound and/or composition of the invention may also be administered directly to the lung by inhalation. For administration by inhalation, a compound and/or composition of the invention may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver compounds of the invention directly to the lung.

Alternatively, a Dry Powder Inhaler (“DPI”) device may be used to administer a compound and/or composition of the invention to the lung. DPI devices typically use a mechanism such as a burst of gas to create a cloud of dry powder inside a container, which may then be inhaled by the patient. DPI devices are also well known in the art. A popular variation is the multiple dose DPI (“MDDPI”) system, which allows for the delivery of more than one therapeutic dose. For example, capsules and cartridges of gelatin for use in an inhaler or insufflator may be formulated containing a powder mix of a compound of the invention and a suitable powder base such as lactose or starch for these systems.

Another type of device that may be used to deliver a compound and/or a composition of the invention to the lung is a liquid spray device. Liquid spray systems use extremely small nozzle holes to aerosolize liquid drug formulations that may then be directly inhaled into the lung.

In one embodiment, a nebulizer is used to deliver a compound and/or composition of the invention to the lung. Nebulizers create aerosols from liquid drug formulations by using, for example, ultrasonic energy to form fine particles that may be readily inhaled (see e.g., Verschoyle et al., British J. Cancer 1999, 80, Suppl. 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974), Aventis and Batelle Pulmonary Therapeutics.

In another embodiment, an electrohydrodynamic (“EHD”) aerosol device is used to deliver a compound and/or composition of the invention to the lung. EHD aerosol devices use electrical energy to aerosolize liquid drug solutions or suspensions (see e.g., Noakes et al., U.S. Pat. No. 4,765,539). EHD aerosol devices may more efficiently deliver drugs to the lung than other pulmonary delivery technologies.

In another embodiment, the compounds of the invention can be delivered in a vesicle, in particular a liposome (see Langer, Science 1990, 249:1527-1533; Treat et al, in “Liposomes in the Therapy of Infectious Disease and Cancer,” Lopez-Berestein and Fidler (eds.), Liss, New York, pp. 353-365 (1989); see generally “Liposomes in the Therapy of Infectious Disease and Cancer,” Lopez-Berestein and Fidler (eds.), Liss, New York, pp. 353-365 (1989)).

In yet another embodiment, the compounds of the invention can be delivered via sustained release systems, preferably oral sustained release systems. In one embodiment, a pump may be used (see Langer, supra; Sefton, 1987, CRC Crit Ref Biomed. Eng. 14:201; Saudek et al., N. Engl. J Med. 1989, 321:574).

In another embodiment, polymeric materials can be used (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Ranger and Peppas, J. Macromol Sci. Rev. Macromol Chem. 1983, 23:61; see also Levy et al., Science 1985, 228: 190; Dunng et al., Ann. Neurol 1989, 25:351; Howard et al., J. Neurosurg. 1989, 71:105). In a preferred embodiment, polymeric materials are used for oral sustained release delivery. In another embodiment, enteric-coated preparations can be used for oral sustained release administration. In still another embodiment, osmotic delivery systems are used for oral sustained release administration (Verma et al., Drug Dev. Ind. Pharm. 2000, 26:695-708).

In yet another embodiment, a controlled-release system can be placed in proximity of the target of the compounds and/or composition of the invention, thus requiring only a fraction of the systemic dose (see, e.g. Goodson, in “Medical Applications of Controlled Release,” supra, vol. 2, pp. 115-138 (1984)). Other controlled-release systems discussed in Langer, 1990, Science 249:1527-1533 may also be used.

5.6. Compositions of the Invention

The present compositions contain a therapeutically effective amount of one or more compounds of the invention, preferably in purified form, together with a suitable amount of a pharmaceutically acceptable vehicle, so as to provide the form for proper administration to a patient. When administered to a patient, the compounds of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents or pH buffering agents. In addition, auxiliary, stabilizing, thickening, lubricating and coloring agents may be used.

Pharmaceutical compositions comprising a compound of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds of the invention into preparations which can be used pharmaceutically. Proper formulation is dependent upon the route of administration chosen.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 17th Edition, 1985).

For topical administration compounds of the invention may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In a preferred embodiment, the compounds of the invention are formulated in accordance with routine procedures as a composition adapted for intravenous administration to human beings. Typically, compounds of the invention for intravenous administration are solutions in sterile isotonic aqueous buffer. For injection, a compound of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. When necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When the compound of the invention is administered by infusion, it can be dispensed, for example, with an infusion bottle containing sterile pharmaceutical grade water or saline. When the compound of the invention is administered by injection, an ampoule of sterile water for injection or saline can be provided so that the ingredients may be mixed prior to administration.

For transmucosal administration, penetrants appropriate to the barrier to be permeated are used in the formulation. Such penetrants are generally known in the art.

Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry coloring agents and preserving agents, to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5.0 mM to about 50.0 mM, etc). Additionally, flavoring agents, preservatives, coloring agents, bile salts, acylcarnitines and the like may be added.

For buccal administration, the compositions may take the form of tablets, lozenges, etc. formulated in conventional manner.

Liquid drug formulations suitable for use with nebulizers and liquid spray devices and EHD aerosol devices will typically include a compound of the invention with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

A compound of the invention may also be formulated in rectal or vaginal compositions such as suppositories or retention enemas, e.g., containing conventional suppository bases such as cocoa butter or other glycerides.

In addition to the formulations described previously, a compound of the invention may also be formulated as a depot preparation. Such long acting formulations may be administered by implantation (e.g., subcutaneously or intramuscularly) or by intramuscular injection. Thus, for example, a compound of the invention may be formulated with suitable polymeric or hydrophobic materials (e.g., as an emulsion in an acceptable oil) or ion exchange resins, or as sparingly soluble derivatives, for example, as a sparingly soluble salt.

When a compound of the invention is acidic, it may be included in any of the above-described formulations as the free acid, a pharmaceutically acceptable salt, a solvate or hydrate. Pharmaceutically acceptable salts substantially retain the activity of the free acid, may be prepared by reaction with bases and tend to be more soluble in aqueous and other protic solvents than the corresponding free acid form.

5.7. Methods of Use And Doses

A compound of the invention, or compositions thereof, will generally be used in an amount effective to achieve the intended purpose. The compounds of the invention or compositions thereof, are administered or applied in a therapeutically effective amount for use to treat or prevent diseases or disorders including but not limited to, ovarian cancer, peritoneal cancer, endometrial cancer, cervical cancer, breast cancer, colorectal cancer, uterine cancer, stomach cancer, small intestine cancer, thyroid cancer, lung cancer, kidney cancer, pancreas cancer, prostrate cancer, acute lung diseases, (e.g., adult respiratory distress syndrome (ARDS) and asthma) surface epithelial cell injury (e.g., transcomeal freezing and cutaneous bums) and cardiovascular diseases such as ischemia and arthesclerosis.

The amount of a compound of the invention that will be effective in the treatment of a particular disorder or condition disclosed herein will depend on the nature of the disorder or condition, and can be determined by standard clinical techniques known in the art as previously described. In addition, in vitro or in vivo assays may optionally be employed to help identify optimal dosage ranges. The amount of a compound of the invention administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration, by multiple applications or controlled release. In a preferred embodiment, the compounds of the invention are delivered by oral sustained release administration. Preferably, in this embodiment, the compounds of the invention are administered twice per day (more preferably, once per day). Dosing may be repeated intermittently, may be provided alone or in combination with other drugs and may continue as long as required for effective treatment of the disease state or disorder.

Suitable dosage ranges for oral administration are dependent on the potency of the, but are generally about 0.001 mg to about 200 mg of a compound of the invention per kilogram body weight. Dosage ranges may be readily determined by methods known to the skilled artisan.

Suitable dosage ranges for intravenous (i.v.) administration are about 0.01 mg to about 100 mg per kilogram body weight. Suitable dosage ranges for intranasal administration are generally about 0.01 mg/kg body weight to about 1 mg/kg body weight. Suppositories generally contain about 0.01 milligram to about 50 milligrams of a compound of the invention per kilogram body weight and comprise active ingredient in the range of about 0.5% to about 10% by weight. Recommended dosages for intradermal, intramuscular, intraperitoneal, subcutaneous, epidural, sublingual or intracerebral administration are in the range of about 0.00 1 mg to about 200 mg per kilogram of body weight. Effective doses may be extrapolated from dose-response curves derived from in vitro or animal model test systems. Animal model systems include, but are not limited to, human tumor xenografts in nude mice. Such animal models and systems are well known in the art (Andersson et al., 2000, Acta Oncl. 39:741-745; Chatzistamou et al., 2001, J. Clin. Endocrinol Metab. 86:2 144-2152).

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred for reducing convulsion. The compounds of the invention may also be demonstrated to be effective and safe using animal model systems.

Preferably, a therapeutically effective dose of a compound of the invention described herein will provide therapeutic benefit without causing substantial toxicity. Toxicity of compounds of the invention may be determined using standard pharmaceutical procedures and may be readily ascertained by the skilled artisan. The dose ratio between toxic and therapeutic effect is the therapeutic index. A compound of the invention will preferably exhibit particularly high therapeutic indices in treating disease and disorders. The dosage of a compound of the inventions described herein will preferably be within a range of circulating concentrations that include an effective dose with little or no toxicity.

5.8. Combination Therapy

In certain embodiments, the compounds of the invention can be used in combination therapy with at least one other therapeutic agent. The compound of the invention and the other therapeutic agent can act additively or, more preferably, synergistically. In a preferred embodiment, a compound of the invention is administered concurrently with the administration of another therapeutic agent. In another preferred embodiment, a composition comprising a compound of the invention is administered concurrently with the administration of another therapeutic agent, which can be part of the same composition as the compound of the invention or a different composition. In another embodiment, a composition comprising a compound of the invention is administered prior or subsequent to administration of another therapeutic agent. Other therapeutic agents, which may be used with the compounds and/or compositions of the invention, include but are not limited to, agonists and antagonists of Edg-4, drugs used to treat cardiovascular diseases and/or cancer such as, alkylating agents (e.g., cyclophosphamide, melphalan, chlorambucil), platinum compounds (e.g., cisplatin, carboplatin), anthracyclines (e.g., doxorubicin, epirubicin), taxanes (e.g., paclitaxel, docetaxel), chronic oral etoposide, topotecan, gemcitabine, hexamethylamine, methotrexate, and 5-fluorouracil.

5.9. Assays

One of skill in the art can use the following assays to identify Edg-4 agonists or antagonists.

5.9.1. Intracellular Calcium Measurement Assays

Specific assays for Edg-4 receptor activity are known to those of skill in the art. For example, cells expressing Edg-4 receptors can be contacted with a membrane-permeant calcium sensitive dye such as Fluo-4 AM or a proprietary calcium dye loading kit (e.g., FLIPR Calcium Assay kit, Molecular Devices, Sunnyvale, Calif.). Intracellular calcium is capable of binding to the dye and emitting fluorescent radiation when illuminated at the appropriate wavelength. The cells can thus be illuminated an appropriate wavelength for the dye and any emitting light can be captured by a cooled CCD camera. Changes in fluorescence indicate changes in intracellular calcium resulting from the activation of an Edg-4 receptor. Such changes can be measured advantageously in whole cells in “real-time” (Berridge et al., Nature Reviews 2000, 1:11-21).

Other methods of measuring intracellular calcium are known to those of skill in the art. For instance, a commonly used technique is the expression of receptors of interest in Xenopus laevis oocytes followed by measurement of calcium activated chloride currents (see Weber, 1999, Biochim Biophys Acta 142 1:213-233). In addition, several calcium sensitive dyes are available for the measurement of intracellular calcium. Such dyes can be membrane permeant or not membrane permeant. Examples of useful membrane permeant dyes include acetoxymethyl ester forms of dyes that can be cleaved by intracellular esterases to form a free acid, which is no longer membrane permeant and remains trapped inside a cell. Dyes that are not membrane permeant can be introduced into the cell by microinjection, chemical permeabilization, scrape loading and similar techniques (Haughland, 1993, in “Fluorescent and Luminescent Probes for Biological Activity” ed. Mason, W. T. pp 34-43; Academic Press, London; Haughland, 1996, in “Handbook of Fluorescent Probes and Research Chemicals”, sixth edition, Molecular Probes, Eugene, Oreg.).

5.9.2. IL-8 and VEGF Assays

The levels of interleukin-8 (“IL-8”) and vascular endothelial growth factor (“VEGF”) are important markers for the proliferative potential, angiogenic capacity and metastatic potential of a tumor cell line. Specific assays for IL-8 and VEGF are known to those of skill in the art. For example, IL-8 and VEGF assays can be performed by techniques that include, but are not limited to, a standard enzyme-linked inimunosorbent assay (“ELISA”). In a standard ELISA, the cells can be cultured, for example, in a 96 well format, serum starved overnight, and treated with LPA or SIP. Dose ranges would be known to one of skill in the art. For example, the doses can range from 0.1-10 μM in serum free medium. Cell supernatants can then be collected to measure the amount of IL-8 or VEGF secreted.

Methods to measure the amount of IL-8 or VEGF secreted are known to one of skill in the art. In one method, an anti-IL-8 or anti-VEGF capture antibody can be adsorbed on to any surface, for example, a plastic dish. Cell supernatants containing IL-8or VEGF can then be added to the dish and any method known in the art for detecting antibodies can be used to detect the anti-IEIL-8 or anti-VEGF antibody. In one embodiment, an anti-IL-8 or anti-VEGF biotinylated detection antibody and streptavidin-HRP can be used for detection via the addition of a substrate solution and calorimetric reading using a microtiter plate reader. The level of IL-8 or VEGF can be interpolated by non-linear regression analysis from a standard curve.

5.9.3. Migration and Invasion Assays

Migration and invasion assays are known to one of skill in the art. For example, migration assays can be designed to measure the chemotactic potential of the cell line, or its movement toward a concentration gradient of chemoattractants, such as, but not limited to, LPA or SIP. Invasion assays can be designed, for example, to evaluate the ability of the cell line to pass through a basement membrane, a key feature of metastasis formation.

Specific assays, known to one of skill in the art include a modified Boyden Chamber assay in which a cell suspension can be prepared in serum free medium and added to the top chamber. The concentration of cells to be added, for example, about 10⁵ cells/ml is known to one of skill in the art. An appropriate dose of a chemoattractant can then be added to the bottom chamber. Following an incubation period, the number of cells invading the lower chamber can be quantified by methods known in the art. In one embodiment, Fluoroblok filter inserts can be used and the number of cells migrating to the lower chamber can be quantified by staining the filter inserts and detecting the fluorescence by any means known in the art. The level of fluorescence may be correlated with the number of migrating cells.

5.9.4. Proliferation Assay

Proliferation assays quantitate the extent of cellular proliferation in response to a stimulant, which, in the case of Edg-4 receptors, may be LPA. Cells can be plated and treated with the stimulant (e.g., LPA) with or without any serum starvation. Stimulant doses may range from 0.1 to 10 μM and in any event may be readily determined by those of skill in the art. Typically, the cells can be treated for a period of a few hours to a few days before cellular proliferation is measured.

Specific methods to determine the extent of cell proliferation are known to one of skill in the art. For example, one method is bioluminescent measurement of ATP, which is present in all metabolically active cells. ATP can be extracted by addition of Nucleotide Releasing Reagent and its release can be monitored by the addition of the ATP Monitoring Reagent. An enzyme, such as luciferase, which catalyzes the formation of light from ATP and luciferin, can be used to quantitate the amount of ATP present.

5.9.5. Cyclic AMP Assay

Because cAMP acts a second messenger in cell signaling, activating protein kinases that in turn phosphorylate enzymes and transcription factors, cAMP concentration is frequently indicative of the activation state of downstream signaling pathways. For GPCRs like the Edg receptors, coupling via a Gui pathway results in inhibition of adenylyl cyclase activity, the key enzyme involved in breakdown of ATP and formation of cAMP. Thus, assays can be designed to measure inhibition of adenylyl cyclase activity, by first stimulating cAMP formation. One example of a compound, which stimulates cAMP formation is forskolin. Forskolin bypasses the receptor and directly activates adenylyl cyclase. Under these conditions, activation of a Gai coupled receptor will inhibit forskolin-stimulated cAMP, and an antagonist at such a receptor will reverse the inhibition.

This assay can be performed by any means known to one of skill in the art. For example, cells can be plated and treated with or without any serum starvation. The cells may be initially treated with a compound, such as forskolin, to induce cAMP production. This is followed by the addition of an Edg-4 stimulator, for example, LPA. The dose of stimulator required is well known in the art, and could be in the range from 0.1-10 αM in serum free medium. Following an incubation period, the cells are lysed and the level of cAMP is determined.

The cAMP assay can be performed by any means known to one of skill in the art, for example, by performing a competitive immunoassay. Cell lysates can be added to a plate precoated with anti-cAMP antibody, along with a cAMP-AP conjugate and a secondary anti-cAMP antibody. Detection can be performed by any appropriate means, including, but not limited to, using a substrate solution and chemiluminescent readout.

### 6. EXAMPLES

The invention is further defined by reference to the following examples, which describe in detail preparation of compounds and compositions of the invention and assays for using compounds and compositions of the invention. It will be apparent to those skilled in the art that many modifications, both to materials and methods, may be practiced without departing from the scope of the invention.

### 6.1. Example 1

Synthesis of 4,4,4-trifluoro-3-oxo-N-(5-phenyl-2H-pyrazol-3-yl)-butyramide (101)

Ethyl 4,4,4-trifluoroacetoacetate (3.45 mL, 23.6 mmol) and acetic acid (5.2 mL) were added to 5-phenyl-1H-pyrazol-3-ylamine (2.5 g, 15.7 mmol). The reaction mixture was heated for 2.5 hours at 120° C., cooled to room temperature, concentrated in vacuo and purified by flash chromatography on silica gel (chloroform/methanol/concentrated aqueous animonium hydroxide) to provide 3.35 g (72% yield) of 101 as a white solid. ¹H NMR (300 MHz, DMSO-d₆) δ: 12.8 (s, 1H), 10.6 (s, 1H), 7.85 (m, 2H), 7.30 (m, 3H), 6.92 (s, 1H), 3.04 (m, 1H), 2.72 (m, 1H). APCI-MS: m/z=298 [C₁₃H,₁₀F₃N₃O₂+H]. Melting range: 318.6-321.1° C. (decomposed).

### 6.2. Example 2

Synthesis of N-[5-(3,4-dichloro-phenyl)-2H-pyrazol-3-yl]4,4,4-trifluoro-3-oxo-butyramide (103)

Thiosemicarbazide (1.15 g, 12.6 mmol) was added to 3′,4′-dichloroacetophenone (2.0 g, 10.6 mmol) in acetic acid (0.12 mL) and ethanol (21 mL) (Dimmock et al., 1991, Eur. J. Med. Chem. 26:529). The reaction mixture was stirred for 4 days at room temperature, concentrated in vacuo and the resultant oil was taken up in chloroform. The chloroform solution was washed successively with saturated aqueous sodium bicarbonate, water and brine, dried with sodium sulfate and concentrated in vacuo to give 2.47 g (89%) of the thiosemicarbazone as a white solid. ¹H NMR (DMSO-d6) δ: 9.7 (br, 1H), 8.37 (s, 1H), 8.28 (m, 1H), 8.22 (s, 1H), 7.89 (m, 1H), 7.13 (m, 1H), 2.24 (s, 3H).

The thiosemicarbazone of 3′,4′-dichloroacetophenone (2.5 g, 9.43 mmol) was added to a solution of lithium diisopropylamnide (39.6 mmol) in THF (20 mL) at 0 ° C. (Beam, et al., 1997, J. Heterocyclic Chem. 34:1549). After two hours at 0° C., aqueous hydrochloric acid (63 mL, 3N) was added and the reaction mixture was heated for 1 hour at 100° C., poured into ice water (200 mL) and neutralized with solid sodium bicarbonate. Extraction of the aqueous mixture with chloroform followed by flash column chromatography on silica gel (4-7% methanol in methylene chloride) provided 1.55 g (72%) of 5-(3,4-dichloro-phenyl)-1H-pyrazol-3-ylamine as a tan foam. ¹H NMR (300 MHz, DMSO-d₆) δ: 11.8 (br, 1H), 7.8 (s, 1H), 7.6 (m, 2H), 5.8 (s, 1H), 4.8 (br, 2H). CI-MS: m/z=228 [C₉H₇Cl₂N₃+H].

Finally, following the procedure of Example 1, 5-(3,4-dichloro-phenyl)-1H-pyrazol-3-ylamine (0.25 g, 1.10 mmol) was reacted with ethyl 4,4,4-trifluoroacetoacetate (0.16 mL, 1.10 mmol) to provide 67 mg (17%) of 103 as a white solid. ¹H NMR (300 MHz, DMSO-d₆) δ: 13.07 (s, 1H), 10.78 (s, 1H), 8.23 (s, 1H), 7.88 (m, 1H), 7.76 (m, 1H), 7.27 (s, 1H), 3.06 (m, 1H), 2.77 (m, 1H). Cl-MS: ml/z =366 [C₁₃H₈Cl₂F₃N₃O₂+H].

### 6.3. Example 3

Synthesis of 4,4-4-trifluoro-N-[5-(4-methoxy-phenyl)-2-pyrazol-3-yl]-3-oxo-butyramide (105)

Following the procedure of Example 1, 5-(4-methoxy-phenyl)-2H-pyrazol-3-ylamine (0.20 g, 1.05 mmol) (Beam, et al., 1997, J. Heterocyclic Chem. 34:1549; Grandin, 1971, Bull. Chim. Soc. Fr. 4002) was reacted with ethyl 4,4,4-trifluoroacetoacetate (0.23 mL, 1.60 mmol) to provide 177 mg (51%) of 105 as a tan solid. ¹H NMR (300 MHz, DMSO-d₆) δ: 12.62 (s, 1H), 10.57 (s, 1H), 7.77 (m, 2H), 6.92 (m, 3H), 3.70 (s, 3H), 2.93 (m, 1H), 2.68 (m, 1H). APCI-MS: m/z=328 [C₁₄H₁₂F₃N₃0₃+H]. Melting Range: 307-310 ° C. (decomposed).

### 6.4. Example 4

Synthesis of 4,4-4-trifluoro-N-15-(4-fluoro-phenyl)2H-Pyrazol-3-yl]-3-oxo-butyramide (107)

Following the procedure of Example 1, 5-(4-fluoro-phenyl)-2H-pyrazol-3-ylamine (0.30 g, 1.69 mmol) (Beam, et al., 1997, J. Heterocyclic Chem. 34:1549; Joshi et al., 1979, J. Heterocyclic Chem. 16:1141) was reacted with ethyl 4,4,4-trifluoroacetoacetate (0.37 mL, 2.54 mmol) to provide 205 mg (39%) of 107 as a white solid. ¹H NMR (300 MHz, DM50-d₆) δ: 12.78 (s, 1H), 10.62 (s, 1H), 7.83 (m, 2H), 7.32 (m, 2H), 6.96 (s, 1H), 2.93 (m, 1H), 2.70 (m, 1H). APCI-MS: m/z=316 [C₁₃H₉F₄N₃O₂+H]. Melting Range: 308-310° C. (decomposed).

### 6.5. Example 5

Synthesis of 2-chloro-4,4,4-trifluoro-3-oxo-N-(5-phenyl-2H-Pyrazol-3-yl)-butyramide (109)

Following the procedure of Example 1, 5-phenyl-1H-pyrazol-3-ylamine (0.25 g, 1.57 mmol) was reacted with ethyl 2-chloro-3-keto-4,4,4-trifluorobutyrate (515 mg, 2.36 mmol) to provide 219 mg (42%) of 109 as a white solid. ¹H NMR (300 MHz, DMSO-d₆) δ: 12.95 (s, 1H), 11.03 (s, 1H), 7.77 (m, 2H), 5.39 (m, 4H), 4.42 (s, 1H). Cl-MS: m/z=332 [Cl3H₉ClF₃N₃O₂+H]. Melting Range: 259-261° C. (decomposed).

### 6.6. Example 6

Synthesis of N-[5-(3,5-dimethoxy-phenyl)-2H-pyrazol-3-yl]-4,4,4-trifluoro-3-oxo-butyramide (111)

Thiosemicarbazide (1.9 g, 20.8 mmol) was added to 3′,5′-dimethoxyacetophenone (2.5 g, 13.9 mmol) following the procedure of Example 2 to give 3.5 g (100%) of the thiosemicarbazone as a white solid. H NMR (300 MHz, DMSO-d₆) δ: 10.2 (s, 1H), 8.3 (s, 1H), 7.9 (s, 1H), 7.0 (s, 2H), 6.5 (s, 1H), 3.7 (s, 6H), 2.3 (s, 3H).

The thiosemicarbazone of 3′,5′-dimethoxyacetophenone (3.5 g, 13.9 mmol) was reacted with base following the procedure of Example 2, to give 2.3 g (75%) of 5-(3,5-dimethoxy-phenyl)-2H-pyrazol-3-ylamine (Beam, et al., 1997, J. Heterocyclic Chem. 34:1549). ¹H NMR (300 MHz, DMSO-d₆) δ: 11.9 (br, 1H), 6.8 (s, 2H), 6.4 (s, 1H), 5.8 (m, 1H), 4.7 (br, 2H), 3.8 (s, 6H).

Finally, following the procedure of Example 1, reaction of 5-(3,5-dimethoxyphenyl)-2H-pyrazol-3-ylamine (0.30 g, 1.37 mmol) with ethyl 4,4,4-trifluoroacetoacetate (0.30 mL, 2.05 mmol) provided 261 mg (53%) of 111 as a white solid. H NMR (300 MHz, DMSO-d₆) 6: 12.83 (s, 1H), 10.78 (s, 1H), 7.14 (s, 2H), 7.08 (s, 1H), 6.52 (s, 1H), 3.77 (s, 6H), 2.99 (m, 1H), 2.78 (m, 1H). APCI-MS: m/z=358 [C₁₅H₁₄F₃N₃O₄+H]. Melting Range: 119-121° C.

### 6.7. Example 7

Synthesis of 4,4,4-trifluoro-N-[5-(3-methoxy-phenyl)-2H-pyrazol-3-yl]-3-oxo-butyramide (113)

Following the procedure of Example 1, 5-(3-methoxy-phenyl)-2H-pyrazol-3-ylamine (0.32 g, 1.70 mmol) (Beam et al, 1997, J. Heterocyclic Chem. 34:1549; Bruni et al., 1993, J. Pharm. Sci. 82:480) was reacted with ethyl 4,4,4-trifluoroacetoacetate (0.37 mL, 2.54 mmol) to provide 183 mg (33%) of 113 as a white solid. ¹H NMR (300 MHz, DMSO-d₆) δ: 12.81 (s, 1H), 10.68 (s, 1H), 7.54 (s, 1H), 7.42(m, 2H), 7.06 (s, 1H), 6.96 (m, 1H), 3.78 (s, 3H), 2.98 (m, 1H), 2.76 (m, 1H). APCI-MS: m/z=328 [C₁₄H₁₂F₃N₃O₃+H]. Melting Range: 107-110° C.

### 6.8. Example 8

Synthesis of N-(5-benzo[31dioxol-5-yl-2H-pyrazol-3-yl) -4.4.4-trifluoro-3-oxo-butyramide (115)

The thiosemicarbazone of 3′,4′-(methylenedioxy) acetophenone (2.6 g, 11.1 mmol) was prepared following the procedure of Example 2 (Dimmock et al., 1991, Eur. J Med. Chem. 26:529). Reaction of the thiosemicarbazone with base following the procedure of Example 2 provided 0.37 g (16%) of 5-benzo[1 ,3]dioxol-5-yl-2H-pyrazol-3-ylamine as an orange foam (Beam, et al., 1997, J. Heterocyclic Chem. 34:1549). ¹H NMR (300 MHz, DMSO-d₆) δ: 11.7 (br, 1H), 7.2 (s, 1H), 7.0 (m, 2H), 6.0 (s, 2H), 5.7 (s, 1H), 4.7 (br, 2H). Cl-MS m/z 204 [C₁₀H₉N₃O₂+H].

Then following the procedure of Example 1, 5-benzo[1,3]dioxol-5-yl-2H-pyrazol-3-ylamine (0.36 g, 1.77 mmol) was reacted with ethyl 4,4,4-trifluoroacetoacetate (0.39 mL, 2.66 mmol) to provide 164mg (27%) of 115 as a white solid. ¹H NMR (300 MHz, DMSO-d₆) δ: 12.69 (s, 1H), 10.67 (s, 1H), 7.48 (s, 1H), 7.36 (m, 1H), 7.02 (m, 2H), 6.09 (s, 2H), 2.96 (m, 1H), 2.77 (m, 1H). APCI-MS: m/z =342 [C₁₄H₁₀F₃N₃O₄+H]. Melting Range: 128-130 ° C.

### 6.9. Example 9

Synthesis of 4,4,4-trifluoro-2-methyl-3-oxo-N-15-phenyl-2H-pyrazol-3-yl]-butyramide (117)

Following the procedure of Example 1, 5-phenyl-1H-pyrazol-3-ylamine (0.25 g, 1.57 mmol) was reacted with ethyl 2-methyl-4,4,4-trifluoroacetoacetate (0.47, 2.36 mmol) to provide 75 mg (15%) of 117 as a white solid. ¹H NMR (300 MHz, DMSO-d₆) δ: 12.67 (s, 1H), 10.51 (s, 1H), 7.78 (m, 2H), 7.41 (m, 3H), 6.58 (s, 1H), 2.64 (m, 1H), 1.15 (m, 3H). CI-MS: m/z =312 [C₁₄H₁₂F₃N₃O₂+H]. Melting Range: 286-288 ° C. (decomposed).

### 6.10. Example 10

Synthesis of 3-phenyl-5-(4,4,4-trifluoro-3-oxo-butyrylamino)-pyrazole-1-carboxylic acid allylamide (119)

4,4,4-trifluoro-3-oxo-N-(5-phenyl-2H-pyrazol-3-yl)-butyramide (101) was reacted with allyl isocyanate (0.09 mL, 1.0 mmol) in DMF (1 mL) at room temperature for 3 hours. Concentration in vacuo and purification by flash column chromatography on silica (chloroform/methanol/concentrated aqueous ammonium hydroxide) provided 190 mg (98%) of 119 as a white solid. ¹H NMR (390 MHz, DMSO-d₆) δ: 9.87 (s, 1H), 8.89 (m, 1H), 8.12 (m, 2H), 7.43 (m, 3H), 7.32 (s, 1H), 5.88 (m, 1H), 5.13 (m, 2H), 3.87 (m, 2H), 3.22 (m, 1H), 2.91 (m, 111). CI-MS: m/z=381 [C,₇H,₅F₃N₄O₃+H]. Melting Range: 114-116 ° C.

### 6.11. Example 11

Synthesis of N-[5-(2-Bromophenyl)-2H-pyrazole-3-yl]-4,4,4-trifluoro-3-oxo-butyramide (133)

A mixture of 2-bromoacetophenone (2 ml, 14 mmol), thiosemicarbazide (2g, 22 mmol), acetic acid (0.17 ml) and methanol (29 ml) was stirred for 16.5 hours at room temperature. The mixture was concentrated in vacuo to obtain 2-bromoacetophenone thiosemicarbazone (3.39 g, 85%). 2-bromoacetophenone thiosemicarbazone was identified by NMR and was used without further purification.

Next, a solution of 2-bromoacetophenone thiosemicarbazone (3.39 g, 12.5 mmol) in tetrahydrofuran (63 ml) was added drop-wise to lithium diisopropylamnide (2M in tetrahydrofuran, 37.3 ml, 74.6 mmol) at 0° C. under nitrogen atmosphere. After the reaction was completed (as analyzed by TLC), the mixture was quenched with hydrochloric acid (3N, 83 ml), and the organic layer was dried and concentrated in vacuo. Following a silica gel chromatography (5-7.5% methanol/dichloromethane), 3-amino-5-(2′-bromophenyl)-2H-pyrazole (0.688 g, 23 %) was obtained as a brown oil and identified by NMR.

Then a mixture of 3-amino-5-(2′-bromophenyl)-2H-pyrazole (0.68 g, 2.86 mmol), ethyl-4,4,4-trifluoroacetoacetate (0.63 ml, 4.29 mmol) and acetic acid (1 ml) was stirred at reflux for 1.5 hours, then cooled to room temperature and concentrated in vacuo. The residue was azeotroped with toluene (70 ml) and the crude product was chromatographed twice on silica gel (20-40% CMA/dichloromethane; CMA=80:18:2 chloroform:methanol:ammonium hydroxide). A tan solid 133 (0.264 g, 25%) was obtained: mp 128-131° C.; ¹H NMR (300 MHz, DMSO-d₆) δ. 12.68. (s, 1H), 10.72 (s, 1H), 7.73 (d, 1H), 7.54 (d, 1H), 7.41 (m, 1H), 6.55 (s, 2H), 2.83 (q, 2H); APCI MS m/z 376 [C₁₃H₉BrF₃N₃O₂+H]⁺.

### 6.12. Example 12

Synthesis of N-[5-(2′,4′-dimethoxyphenyl)-2H-pyrazole-3-yl]-4,4,4-trifluoro-3-oxo-butyramide (135)

A mixture of 2,4-dimethoxyacetophenone (2.5 g, 13.9 mmol), thiosemicarbazide (1.9 g, 20.8 mmol), acetic acid (0.159 ml) and methanol (28 ml) was heated for five days with stirring. The mixture was cooled to room temperature and concentrated in vacuo to obtain 2,4-dimethoxyacetophenone thiosemicarbazone (4.29 g, >100%) as a tan solid. 2,4-dimethoxyacetophenone thiosemicarbazone was identified by NMR and was used without further purification.

Next, a solution of 2,4-dimethoxyacetophenone thiosemicarbazone (4.29 g, 16.9 mmol) in tetrahydrofuran (85 ml) was added drop-wise to lithium diisopropylamide (2M in tetrahydrofuran, 59 ml, 199 mmol) at ambient temperature under nitrogen atmosphere. After the reaction was completed (as analyzed by TLC), the mixture was quenched with hydrochloric acid (4N, 113 ml), and the organic layer was dried and concentrated in vacuo. Following a silica gel chromatography (5-7.5 % methanol/dichloromethane), 3-amino-5-(2′,4′-dimethoxyphenyl)-2H-pyrazole (2.36 g, 64%) was obtained as a yellow-brown solid and identified by NMR.

Then a mixture of 3-amino-5-(2′,4′-dimethoxyphenyl)-2H-pyrazole (0.3 g, 1.37 mmol), ethyl-4,4,4-trifluoroacetoacetate (0.3 ml, 2.05 mmol) and acetic acid (0.5 ml) was stirred at reflux for 1.75 hours, then cooled to room temperature and concentrated in vacuo. The residue was azeotroped with toluene (70 ml) and the resulting residue was chromatographed on silica gel (20-40 % CMA/dichloromethane; CMA=80:18:2 chloroform:methanol:ammonium hydroxide), followed by a second silica gel chromatography (30-45 % methanol/dichloromethane). A white solid 135 (0.245 g, 50%) was obtained: mp 125-128° C.; ¹H NMR (300 MHz, DMSO-d₆) δ12.35 (s, 1H), 10.61 (s, 1H), 7.46 (d, 1H), 6.65 (s, 1H), 6.58 (d, 1H), 6.45 (s, 1H), 3.82 (s, 3H), 3.75 (s, 3H), 2.81 (q, 2H); CI MS m/z 358 [C₁₅H₁₄F₃N₃O₄+H]⁺.

### 6.13. Example 13

Synthesis of 4,4,4-trifluoro-3-oxo-N-(5-thiophen-2-yl-2H-pyrazol-3-yl)butyramide (137)

A mixture of 2-amino-5-(2′-thienyl)-2H-pyrazole (0.25 g, 1.52 mmol), ethyl-4,4,4-trifluoroacetoacetate (0.332 ml, 2.28 mmol) and acetic acid (0.5 ml) was stirred at reflux for 1.5 hours and cooled to room temperature. The mixture was concentrated in vacuo and the residue was chromatographed on silica gel (20-40 % CMA/dichloromethane; CMA=80:18:2 chloroform:methanol:ammonium hydroxide). A white solid 137 (0.332 g, 72 %) was obtained: mp 259-261° C.; ¹H NMR (300 MHz, DMSO-d₆) 6.12.95 (bs, 1H), 10.78 (bs, 1H), 7.75 (d, 2H), 7.15 (s, 1H), 7.08 (s, 1H), 3.01 (d, 1H), 2.78 (d, 1H); CI MS m/z 304 [C₁₁H₈F₃N₃O₂S+H]⁺.

### 6.14. Example 14

Synthesis of 4,4,4-trifluoro-3-oxo-N-(5-thiophen-3-yl-2H-pyrazol-3-yl)butyramide (139)

A mixture of 3-acetylthiophene (2.5 g, 19.8 mmol), thiosemicarbazide (2.7 g, 29.7 mmol), acetic acid (0.226 ml) and methanol (40 ml) was stirred at 70° C. for 14 hours and cooled to room temperature. The mixture was concentrated in vacuo, and 3-acetylthiophene thiosemicarbazone (3.92 g, 100 %) was obtained as a pale yellow solid. The compound was identified by NMR.

Next, a solution of 3-acetylthiophene thiosemicarbazone (2 g, 10.1 mmol) in tetrahydrofuran (50 ml) was added drop-wise to lithium diisopropylamide (2M in tetrahydrofuran, 30 ml, 60 mmol) at 0C under nitrogen atmosphere. After the reaction was completed (as analyzed by TLC), the mixture was qhenched with hydrochloric acid (3N, 67 ml), and the organic layer was dried using magnesium sulfate and concentrated in vacuo. The residue was chromatographed on silica gel (4-8 % methanol/dichloromethane), and 3-amino-5-(3-thienyl)-2H-pyrazole (1 g, 60%) was obtained as a brown oil. The compound was identified by NMR.

Then a mixture of 3-amino-5-(3-thienyl)-2H-pyrazole (0.9 g, 5.45 mmol), ethyl-4,4,4-trifluoroacetoacetate (1.2 ml, 8.2 mmol) and acetic acid (2 ml) was stirred for 1.5 hours at reflux, and then cooled to room temperature. The mixture was concentrated in vacuo and azeotroped with toluene. The residue was chromatographed on silica gel (20-60% CMA/dichloromethane; CMA=80:18:2 chloroform:methanol:ammonium hydroxide). A tan solid 139 (0.119 g, 7.2 %) was obtained: mp 251-253° C.; ¹H NMR (300 MHz, DMSO-d₆) δ 12.84 (s, 1H), 10.61 (s, 1H), 8.19 (s, 1H), 7.75 (s, 1H), 7.70 (s, 1H), 7.12 (s, 1H), 3.01 (d, 1H), 2.79 (d, 1H); CI MS m/z 304 [C₁₁H₈F₃N₃O₂S+H]⁺.

### 6.15. Example 15

Synthesis of 4,4,4-trifluoro-3-oxo-N-(5-pyridin-4-yl-2H-pyrazol-3-yl)butyramide (141)

Hydrazine (0.6 ml, 19 mmol) was added to a mixture of cyanoacetyl-4-pyridine (1.39 g, 9.52 mmol) in acetic acid (6 ml). The addition resulted in an exotherm and the mixture was heated for 2.5 hours. The mixture was then cooled to room temperature and diluted with 37 ml of water. Concentrated hydrochloric acid (0.16 ml) was added, and the mixture was heated for 0.5 hour. The mixture was again cooled to room temperature and filtered. N-(5-pyridin-4-yl-2H-pyrazol-3-yl)acetamide (1 g, 50%) was obtained as an orange solid. The compound was identified by NMR and mass spectral analyses.

Next, a mixture of N-(5-pyridin-4-yl-2H-pyrazol-3-yl)acetamide (1 g, 4.95 mmol) and hydrochloric acid (1N, 20 ml) was heated for 4 hours, and then cooled to room temperature and filtered with water wash. The filtrate was neutralized with saturated sodium bicarbonate and extracted three times with methylene chloride. The combined extracts were dired using sodium sulfate and concentrated in vacuo. 3-amino-5-(4-pyridyl)-2H-pyrazole (0.122 g, 15%) was obtained as a yellow solid. The compound was identified by NMR.

Then a mixture of 3-amino-5-(4-pyridyl)-2H-pyrazole (0.122 g, 0.76 mmol), ethyl-4,4,4-trifluoroacetoacetate (0.167 ml, 1.14 mmol) and acetic acid (0.5 ml) was stirred at reflux for 1.5 hours and then cooled to room temperature. The mixture was diluted with toluene and concentrated in vacuo. The residue was chromatographed twice on silica gel (25-75% CMA/dichloromethane then 40% CMA/dichloromethane; CMA=80:18:2 chloroform:methanol:ammonium hydroxide). A yellow solid 141 (48.8 mg, 21.6 %) was obtained: mp 364-366° C.; ¹H NMR (300 MHz, DMSO-d₆) δ 13.13 (s, 1H), 10.76 (s, 1H), 8.69 (s, 2H), 7.98 (s, 2H), 7.25 (s, 1H), 3.17 (d, 1H), 2.80 (d, 1H); CI MS m/z 299 [C₁₂H₉F₃N₄O₂+H]⁺.

### 6.16. Example 16

Synthesis of 5-p-Tolyl-1H-imidazole-2-thiol (143)

2-oxo-2-p-tolyl-ethylammonium chloride was prepared according to the procedures described in Synthesis, pp. 615-618 (1990). Starting from 2-bromo-4′-methylacetophenone (Aldrich), a substitution reaction by sodium diformylamide (TCI-US), followed by an acidic hydrolysis, was performed to provide 95% yield.

2-oxo-2-p-tolyl-ethylammonium chloride (37.74 g, 0.203 mol), KSCN (Acros, 21.84 g, 0.225 mol, 1.1 equivalent) in glacial acetic acid (500 ml) were stirred at 120-125° C. (oil bath temperature) for 2 hours (J. Ind. Chem. Soc., 58:1117-1118 (1981)). The content was then cooled to room temperature, and water (500 ml) was added. The mixture was chilled with an ice bath for 1 hour. The solid product was collected by suction filtration, washed with water, and air-dried. 5-p-tolyl-1H-immidazole-2-thiol was obtained as a yellow solid (37.17 g, 96 %): ¹H NMR (300 MHz, DMSO-d₆) δ 12.47 (s, 1H), 12.10 (s, 1H), 7.56 (m, 2H), 7.32 (s, 1H), 7.18 (m, 2H), 2.29 (s, 3H); APCI-MS m/z 191 [C₁₀H₁₀N₂S+H]⁺; m.p. 266-267° C.

### 6.17. Example 17

Synthesis of 5-(1H-Indol-3-yl)-6-phenyl-4,5-dihydro-2H-[1,2,4]triazin-3-one (121)

Compound 121 was synthesized using the procedures disclosed in Russ, J. Org. Chem. 36: 626-628 (2000. A mixture of the 6-phenyl-1,2,4-triazin(2H)one (0.097 g, 0.56 mmol), indole (0.066 g, 0.56 rnmol) and acetic acid (2 ml) was stirred at reflux for 12 hours. The acetic acid was removed in vacuo. Water (10 ml) was added to form a white precipitate, and the precipitate was filtered and washed with water. Recrystallization from methanol provided 5-(1H-Indol-3-yl)-6-phenyl-4,5-dihydro-2H-[-1,2,4]triazin-3-one (0.14 g, 86%) as a white solid: mp 281 ° C.; ¹H NMR (500 MHz, CD₃OD) δ 7.70 (t, 3H), 7.32 (d, 1H), 7.28 (d, 3H), 7.19 (s, 1H), 7.10 (t, 1H), 7.05 (t, 1H), 6.02 (s, 1H); ESI MS m/z 291 [C₁₇H₁₄N₄O+H]⁺.

### 6.18. Example 18

Resolution of (+) and (−) Isomers of 5-(1H-Indol-3-yl)-6-phenyl-4,5-dihydro-2H-[1,2,4]triazin-3-one (121)

The (+) and (−) isomers of compound 121 were resolved by a chromatographic method. The chromatography was done using Chiralpak AD 50×500 mm column and 60:40 2-propanol/hexane at a flow rate of 118 ml/min.

(+)-5-(1H-Indol-3-yl)-6-phenyl-4,5-dihydro-2H-[1,2,4]triazin-3-one (0.043 g, [α]²⁵_{D}+78.5° (c 0.169, THF)) was recovered as a white solid: mp 281° C.; ¹H NMR (500 MHz, CD₃OD) δ 7.73 (t, 3H), 7.35 (d, 1H), 7.29 (s, 3H), 7.20 (s, 1H), 7.11 (t, 1H), 7.05 (t, 1H), 6.03 (s, 1H); ESI MS m/z 291 [C₁₇H₁₄N₄O+H]⁺.

(−)-5-(1H-Indol-3-yl)-6-phenyl-4,5-dihydro-2H-[1,2,4]triazin-3-one (0.038 g, [α]²⁵_{D} −71.5° (c 0.186, THF)) was also recovered from the same chromatography as a white solid: mp 281° C.; ¹H NMR (500 MHz, CD₃OD) δ 7.76 (m, 3H), 7.36 (d, 1H), 7.28 (s, 3H), 7.21 (s, 1H), 7.16 (t, 1H), 7.09 (t, 1H), 6.04 (s, 1H); ESI MS m/z 291 [C₁₇H₁₄N₄O+H]⁺.

### 6.19. Example 19

Synthesis of 1-(2,6-dichlorophenyl)-6,7-dimethoxy-1,4-dihydro-2H-isoguinolin-3-one (125)

3,4-Dimethoxyphenyl acetonitrile (3.54 g, 20 mmol) was added to polyphosphoric acid (11.1 g) preheated to 130° C. After 1 hour, 2,6-dichlorobenzaldehyde (1.75 g, 20 mmol) was added. The resulting mixture was stirred for 12 hours and cooled to ambient temperature. Following an addition of water (50 ml), concentrated ammonium hydroxide was added. The mixture was allowed to stand for 18 hours. The solids were filtered, then stirred at reflux in sodium hydroxide (1.35 M, 50 ml) for 2 hours. The mixture was filtered while hot, and the solid was washed with water and dried. The solid was chromatographed (silica gel, 5 to 50% ethyl acetate/dichloromethane) to provide 1-(2,6-dichlorophenyl)-6,7-dimethoxy-1,4-dihydro-2H-isoquinolin-3-one (0.305 g, 9 %) as a slightly yellow solid: m.p. 228-229° C.; ¹H NMR (500 MHz, CD₃OD) δ 7.47 (bs, 2H), 7.36 (t, 1H), 6.79 (s, 1H), 6.70 (s, 1H), 6.38 (s, 1H), 3.87 (s, 3H), 3.69 (d, 2H), 3.62 (s, 3H); ESI-MS m/z 352 [C₁₇H₁₅C₁₂NO₃+H]⁺.

### 6.20. Example 20

Synthesis of 3-(2-chloro-6-fluorophenyl)-6-trifluoromethyl-[1,2,4]triazolo[4,3-a]pyridine (127)

1-(3-Dimethylaminopropyl)-3-ethylcarbodiimide hydrochloride (EDC.HCl, 1.32 g, 6.91 mmol), 4-methylmorpholine (0.76 ml, 6.91 mmol), 1-hydroxybenzotriazole hydrate (HOBt, 0.132 g, 0.98 mmol) and 5-trifluoromethylpyridyl hydrazine (68%, 1.5 g, 5.76 mmol) were added to 2-chloro-6-fluorobenzoic acid (1.0 g, 5.76 mmol) in anhydrous 1:1 dichloromethane/acetonitrile (40 ml) at 0° C. The ice bath was removed and the mixture was stirred at ambient temperature for 60 hours. The reaction mixture was concentrated in vacuo, diluted with dichloromethane (120 ml), washed with water (three times, 30 ml each) and brine (40 ml), dried with magnesium sulfate, and concentrated in vacuo. Following a flash silica gel chromatography (5-33% ethyl acetate/dichloromethane), 2-chloro-6-fluorobenzoic acid-N-(5-trifluoromethylpyridin-2-yl)hydrazide (0.542 g, 28%) was obtained as a green solid. The compound was identified by NMR spectral analysis.

Next, phosphorous oxychloride (3.0 ml, 32.4 mmol) was added to a solution of 2-chloro-6-fluorobenzoic acid-N-(5-trifluoromethyl-pyridin-2-yl)hydrazide (0.542 g, 1.62 mmol) in anhydrous toluene (40 ml). The mixture was stirred at reflux for 18 hours. The mixture was then poured into cold sodium hydroxide (2M, 100 ml), extracted with ethyl acetate, washed with water (35 ml) and brine (35 ml), dried with magnesium sulfate. Extra solvent was removed in vacuo. Following a flash silica gel chromatography (5-50% ethyl acetate/dichloromethane), 3-(2-chloro-6-fluorophenyl)-6-trifluoromethyl-[1,2,4]triazolo[4,3-a]pyridine 127 (0.117 g, 23%) was obtained as a light yellow solid: mp 170-171° C.; ¹H NMR (500 MHz, CD₃OD) δ 8.60 (s, 1H), 8.10 (d, 1H), 7.80 (m, 2H), 7.65 (d, 1H), 7.45 (t, 1H); ESI MS m/z 316 [C₁₃H₆ClF₄N₃+H]⁺.

### 6.21. Example 21

Synthesis of 3-(2,3-dichlorophenyl)-6-trifluoromethyl[1,2,4]triazolo[4,3-a]pyridine (129)

1-(3-Dimethylaminopropyl)-3-ethylcarbodiimide hydrochloride (EDC.HCl, 1.325 g, 6.91 mmol), N-methylmorpholine (0.76 ml, 6.91 mmol), 1-hydroxybenzotriazole hydrate (HOBt, 0.177 g, 1.31 mmol) and 5-trifluoromethylpyridyl hydrazine (68%, 1.0 g, 3.84 mmol) were added to 2,3-dichlorobenzoic acid (1.0 g, 5.76 mmol)in anhydrous 1:1 dichloromethane/acetonitrile (40 ml) at 0° C. The ice bath was removed and the mixture was stirred at ambient temperature for 60 hours. The reaction mixture was then concentrated in vacuo, diluted with dichloromethane (100 ml), washed with water (three times, 30 ml each) and brine (40 ml), dried over magnesium sulfate, and concentrated in vacuo. Following a flash silica gel chromatography (5-20% ethyl acetate/dichloromethane), 2,3-dichlorobenzoic acid-N-(5-trifluoromethyl-pyridin-2-yl)hydrazide (0.6 g, 40%) was obtained as a solid. The compound was identified by NMR spectral analysis.

Next, phosphorous oxychloride (3.2 ml, 34.3 mmol) was added to a solution of 2,3-dichlorobenzoic acid-N-(5-trifluoromethylpyridin-2-yl)hydrazide (0.6 g, 1.71 mmol) in anhydrous toluene (40 ml). The resulting mixture was stirred at reflux for 21 hours. The mixture was then poured into cold aqueous sodium hydroxide (2M, 100 ml), extracted with ethyl acetate, washed with water (40 ml) and brine (40 ml), dried over magnesium sulfate, and concentrated in vacuo. Following a flash silica gel chromatography (5-50% ethyl acetate/dichloromethane), a yellow solid was obtained. The yellow solid was again chromatographed on silica gel (5-33% ethyl acetate/hexanes), and 3-(2,3-dichlorophenyl)-6-trifluoromethyl-[1,2,4]triazolo[4,3-a]pyridine 129 (245 mg, 45%) was obtained as an off-white solid: mp 100-101° C.; ¹H NMR (500 MHz, CD₃OD) δ 8.59 (s, 1H), 8.08 (d, 1H), 7.95 (d, 1H), 7.79 (d, 1H), 7.72 (d, 1H), 7.67 (m, 1H); ESI MS m/z 332 [C₁₃H₆C₁₂F₃N₃+H]⁺.

### 6.22. Example 22

Synthesis of 3-(2,6-dichlorophenyl)-6-trifluoromethyl-[1,2,4]triazolo [4,3-a]pyridine (131)

1 -(3-Dimethylaminopropyl)-3-ethylcarbodiimide hydrochloride (EDC.HCl, 1.325 g, 6.91 mmol), 4-methylmorpholine (0.76 ml, 6.91 mmol), 1-hydroxybenzotriazole hydrate (HOBt, 0.177 g, 1.31 mmol) and 5-trifluoromethylpyridyl hydrazine (68%, 1.0 g, 3.84 mmol) were added to 2,6-dichlorobenzoic acid (1.0 g, 5.76 mmol) in anhydrous 1:1 dichloromethane/acetonitrile (40 ml) at 0° C. The ice bath was removed and the mixture was stirred at ambient temperature for 60 hours. The reaction mixture was concentrated in vacuo, diluted with dichloromethane (100 ml), washed with water (three times, 30 ml each) and brine (40 ml), dried with magnesium sulfate, and concentrated in vacuo. Following a flash silica gel chromatography (5-50% ethyl acetate/dichloromethane), 2,6-dichlorobenzoic acid-N-(5-trifluoromethylpyridin-2-yl)hydrazide (0.732 g, 36%) was obtained as a solid. The compound was identified by NMR spectral analysis.

Next, phosphorous oxychloride (3.9 ml, 41.8 mmol) was added to a solution of 2,6-dichlorobenzoic acid-N-(5-trifluoromethyl-pyridin-2-yl)hydrazide (0.732 g, 2.09 mmol) in anhydrous toluene (40 ml). The mixture was stirred at reflux for 18 hours. The mixture was then poured into cold aqueous sodium hydroxide (2M, 100ml), extracted with ethyl acetate, washed with water (40 ml) and brine (40 ml), dried over magnesium sulfate, and concentrated in vacuo. Following a flash silica gel chromatography (5-25% ethyl acetate/hexanes), 3-(2,6-dichlorophenyl)-6-trifluoromethyl-[1,2,4]triazolo[4,3-a]pyridine 131 (0.1 g, 14%) was obtained as an off-white solid: mp 104-105° C.; ¹H NMR (500 MHz, CD₃OD) 6 8.61 (s, ¹H), 8.10 (d, ¹H), 7.78 (d, 1H), 7.72 (s, 3H); ESI MS m/z 332 [C₁₃H₆Cl₂F₃N₃+H]⁺.

### 6.23. Example 23

### Inhibition of the Ed2-4 Receptor by Compound 101

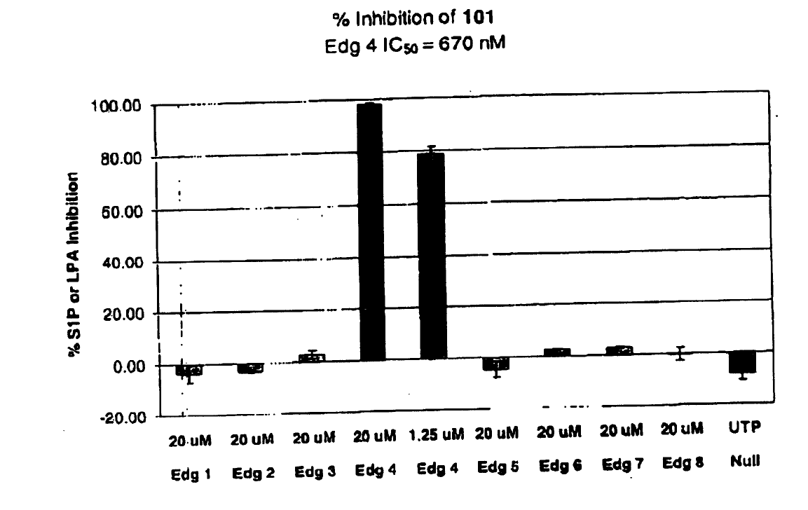

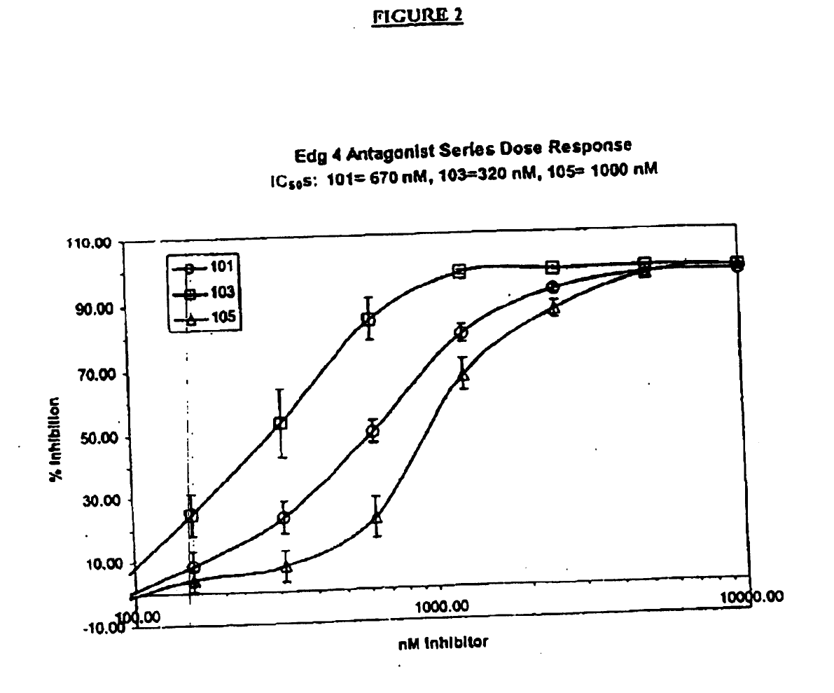

FIG. 1 demonstrates that compound 101 specifically inhibited the Edg 4 receptor. Compound 101 did not inhibit LPA-stimulated calcium increases in HTC cells expressing Edg 2 or Edg 7 receptors and also did not inhibit SIP-stimulated calcium increases in HTC cells expressing Edg 1, Edg 3, Edg 5, Edg 6, or Edg 8 receptors. When tested with the Edg 4 receptor, compound 101 almost completely blocked the LPA response in concentrations between about 1 [M and about 10 μM. FIG. 2 shows that compound 103 has a 2-3 fold greater potency than compound 101, while compound 105 is less potent than compound 101.

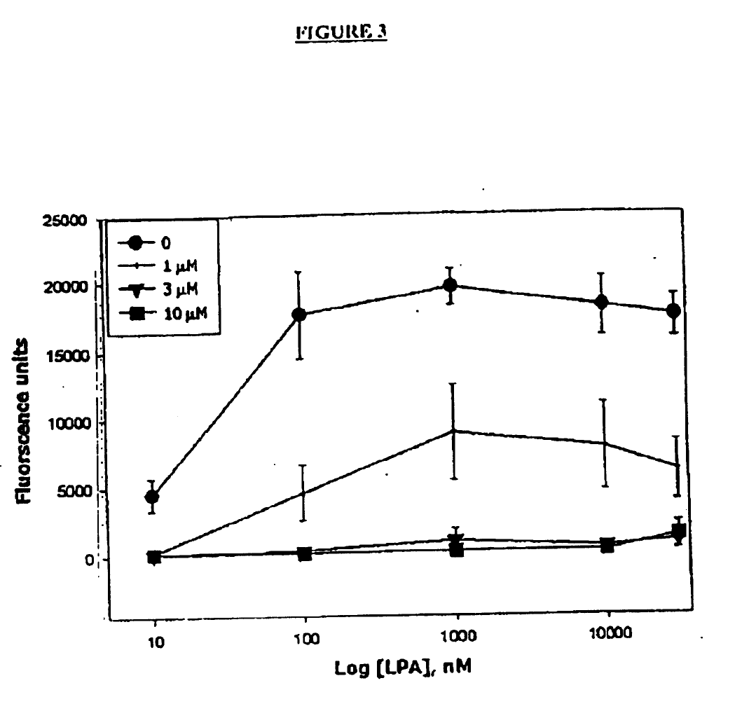

FIG. 3 illustrates a dose response to LPA using varying concentrations of 101 (0-10 μM) in HTC cells expressing human Edg 4 receptors. The data suggests that inhibition by compound 101 may be non-competitive, as demonstrated by the inability of LPA to overcome inhibition by compound 101 at concentrations as high as 10 μM.

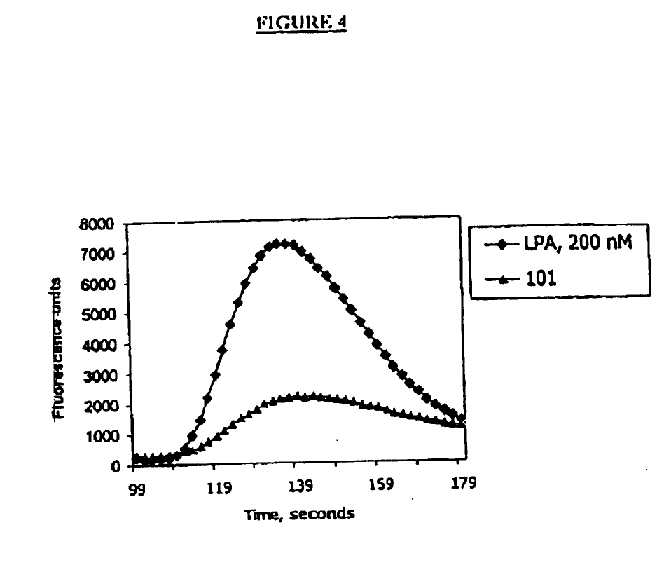

FIG. 4 demonstrates that compound 101 retained its activity when tested on endogenous Edg 4 receptors from human ovarian cancer cells (OV202). LPAstimulated calcium responses in these cells was almost completely inhibited by 10 μM of compound 101. The calcium mobilization assays were conducted as described in Section 6.26 (Example 26).

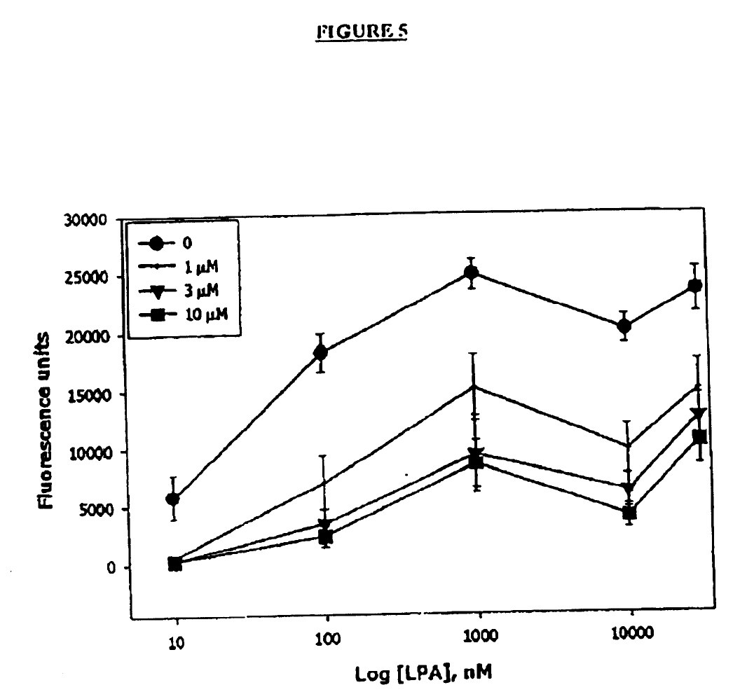

Compound 101 also inhibited LPA-stimulated calcium response in another human ovarian cancer cell line, CaOV3, in a non-competitive mode (FIG. 5). In this instance, the LPA response was not completely inhibited, because these cells express other LPA receptors (Edg 2 and Edg 7).

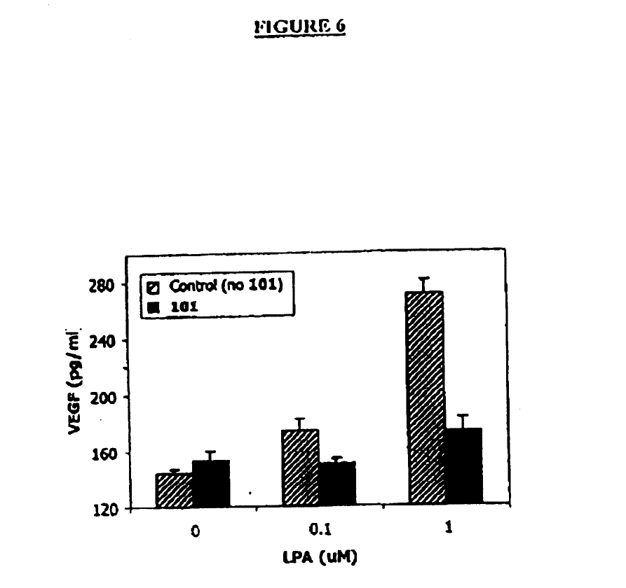

Vascular Endothelial Growth Factor, (“VEGF”), is a potent mitogenic and highly angiogenic factor that causes vascular permeability, which leads to ascites formation. Furthermore, VEGF is tumor-specific. Plasma VEGF levels are significantly elevated in patients with various tumors, including prostate and ovarian cancer (George et al., 2001, Clin Cancer Res 7:1932-1936; Hu et al., 2001, Natl. Cancer Inst. 93 (10):762-767). Therefore, the ability of Edg-receptor antagonists to block VEGF secretion from tumor cells is a particularly relevant secondary assay for potential anti-tumor therapies. FIG. 6 shows that compound 101 completely blocked LPA-stimulated VEGF production in CaOV3 human ovarian cancer cells. The VEGF assays were conducted as described in Section 6.27 (Example 27).

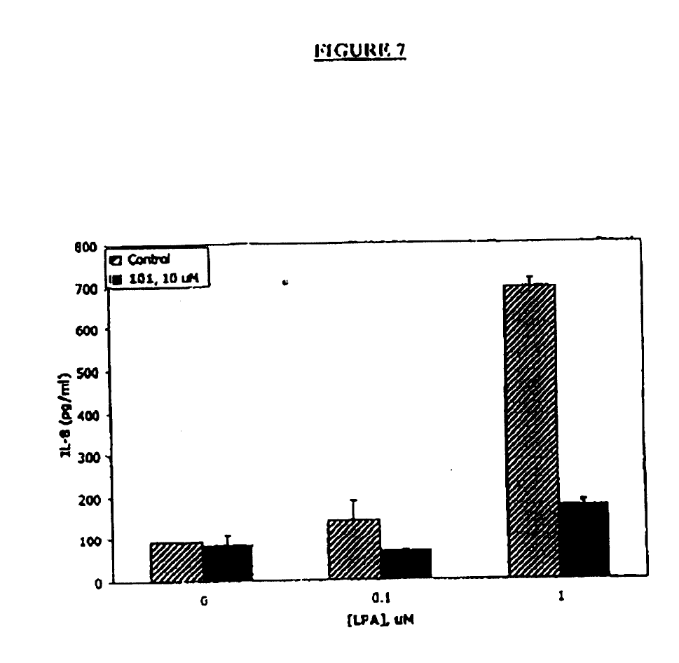

Ovarian cancer cells are known to increase IL-8 secretion (Schwartz et al., 2001, Gynecol. Oncol. 81 (2):291-300). Further, expression of IL-8 has been correlated with cell metastatic potential (Singh et al., 1994, Cancer Res. 54(12):3242-3247). In addition to blocking production of VEGF, compound 101 also completely blocked the production of IL-8 in CaOV3 human ovarian cancer cells (FIG. 7). The IL-8 assays were conducted as described in Section 6.27 (Example 27).

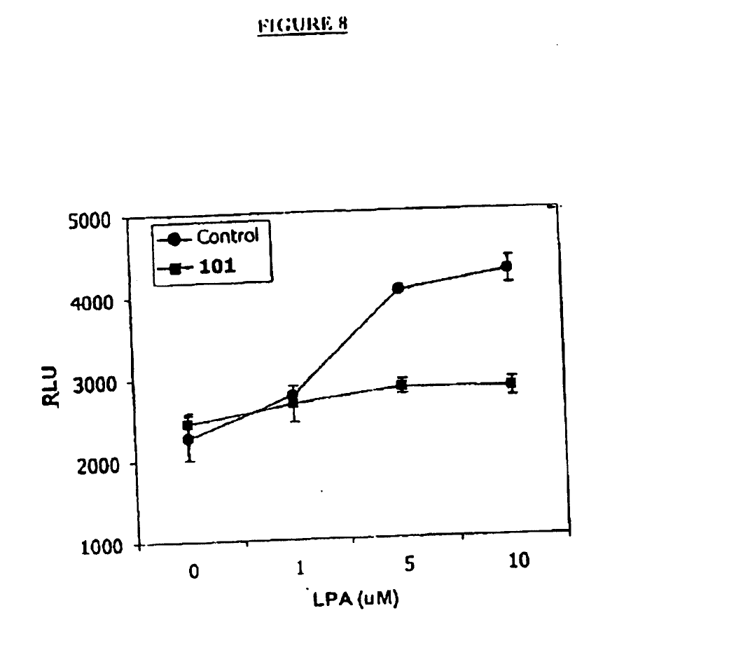

Since LPA is a potent mitogen, it was important to establish whether blocking Edg 4 in human ovarian cancer cells would also block proliferation. Compound 101 (10 μM) effectively abolished LPA-stimulated proliferation of CaOV3, human ovarian cancer cells over a period of 24 hours (FIG. 8). The proliferation assays were conducted as described in Section 6.29 (Example 29).

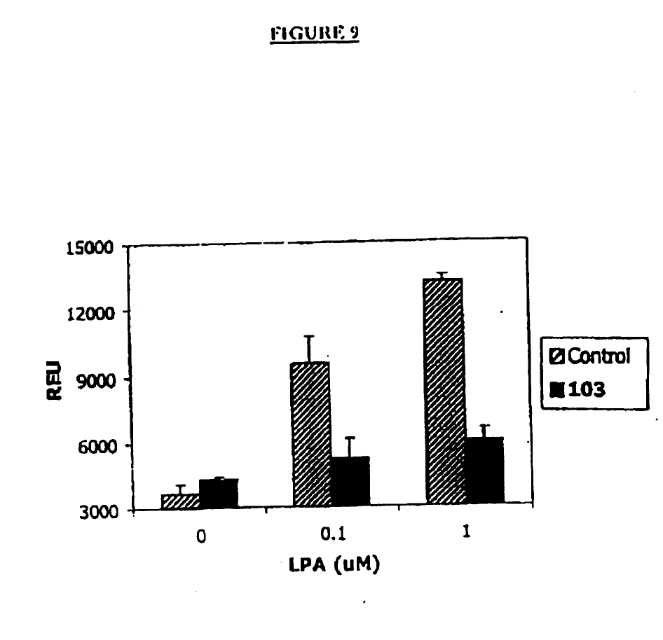

LPA-stimulated chemotaxis is another important marker for angiogenesis and metastasis. FIG. 9 demonstrates that LPA stimulated chemotaxis in CaOV3 human ovarian cancer cells was effectively blocked by Edg 4 antagonist 103.

### 6.24. Example 24

Selective Inhibition of the Edg-4 Receptor by Compounds 101 and 103

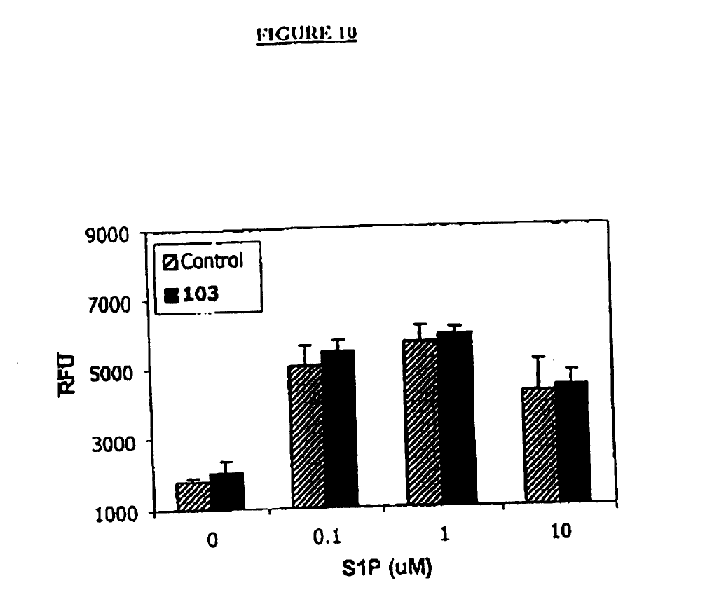

Selectivity of the illustrative compounds 101 and 103 for Edg-4 was demonstrated in several ways. First, compound 101 did not demonstrate any inhibitory activity at any of the other Edg receptors tested (FIG. 1). Second, compound 103 did not inhibit SIP induced chemotaxis in HUVEC cells (FIG. 10), although it did inhibit LPA-stimulated chemotaxis in CaOV3 cells (FIG. 9), which is mediated by the Edg-4 receptor. Third, compound 101 did not demonstrate any significant activity at various targets tested, including other Edg receptors, GPCRs, ion channels, and enzymes (Tables 1 and 2). Table 1 demonstrates the selectivity of compounds 101 and 103 for Edg-4 relative to other Edg receptors and Table 2 is a list of targets, including GPCRs and ion channels, for which compound 101 showed no significant activity in radioligand binding assays. All radioligand binding assays used 10 μM of 101 unless otherwise noted (numbers in parenthesis refer to concentrations in certain assays that were higher than 10 μM). The radioligand binding assays were conducted as described in Section 6.31 (Example 31).

TABLE 1
Selectivity of 101 and 103 for Edg-4

| | 101 | 103 |
|---|---|---|
| Edg-1 | >20 | >20 |
| Edg-2 | >20 | >20 |
| Edg-3 | >20 | >20 |
| Edg-4 | 0.67 | 0.32 |
| Edg-5 | >20 | >20 |
| Edg-6 | >20 | >20 |
| Edg-7 | >20 | >20 |
| Edg-8 | >20 | >20 |
| Fold-selectivity | >29.9 | >62.5 |

(Measurements of IC₅₀, unit=μM)

TABLE 2
Pharmacology Profiling of 101

| | |
|---|---|
| Phosphodiesterases (100 μM) | Cannabinoid |
| PDE₁ | CB1 |
| PDE₂ | Dopamine |
| PDE₃ | D1 |
| PDE₄ | D2L |
| PDE₅ | GABA_{A}, agonist site |
| PDE₆ | Glutamate, NMDA, Phencyclidine |
| | Histamine |
| A. Phospholipases | H1 |
| PLA₂-I (300 μM) | Imidazoline |
| PLA₂-II (300 μM) | I2 |
| PLC | Muscarinic |
| | M2 |
| Adenosine | Nicotinic acetylcholine, central |
| A₁ | Opiate |
| A_{2A} | M |
| Adrenergic | Phorbol ester |
| α1A | PDGF |
| α2A | Potassium channel [KATP] |
| β1 | Sigma |
| β2 | σ1 |
| Norepinephrine transporter | σ2 |
| Calcium channel | Sodium channel, site 2 |
| Dihydropyridine | VEGF |

(Measurements of IC₅₀, unit=μM)

Table 3 illustrates the selectivity of the Edg-4 antagonist 121 for Edg-4 relative to other Edg receptors. Similarly, selectivity of Edg-4 agonists 125, 129, and 131 is summarized in Table 3. As shown in Table 3, the compounds of this invention show higher efficacy for Edg-4 receptors than other Edg receptors. It should also be noted that compound 125 also acts as an agonist against the Edg-7 receptor.

TABLE 3
Selectivity of Various Compounds for Edg-4 Receptors

| | 121 | | | |
|---|---|---|---|---|
| | (IC₅₀, μM) | 125 (EC₅₀, μM) | 129 (EC₅₀, μM) | 131 (EC₅₀, μM) |
| Edg 1 | >20 | >25 | >25 | >25 |
| Edg 2 | >20 | >25 | >25 | >25 |
| Edg 3 | >20 | >25 | >25 | >25 |
| Edg 4 | 3.6 | 5.2 | 5.4 | 9.9 |
| Edg 5 | >20 | >25 | >25 | >25 |
| Edg 6 | >20 | >25 | >25 | >25 |
| Edg 7 | >20 | 2.5 | >25 | >25 |
| Edg 8 | >20 | >25 | >25 | >25 |
| Null | — | >25 | >25 | >25 |
| Fold | >5.6 | >4.8 | >4.6 | >2.5 |
| Selectivity | | | | |

### 6.25. Example 25

Specificity of Compounds on the Ed2-4 Receptor

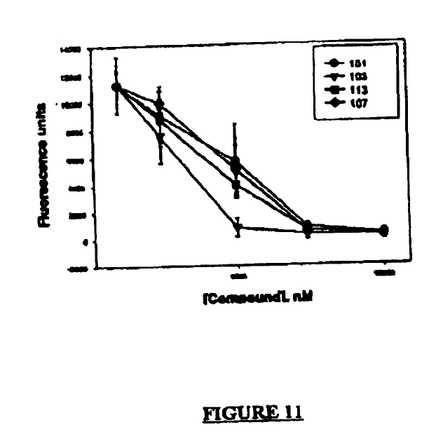

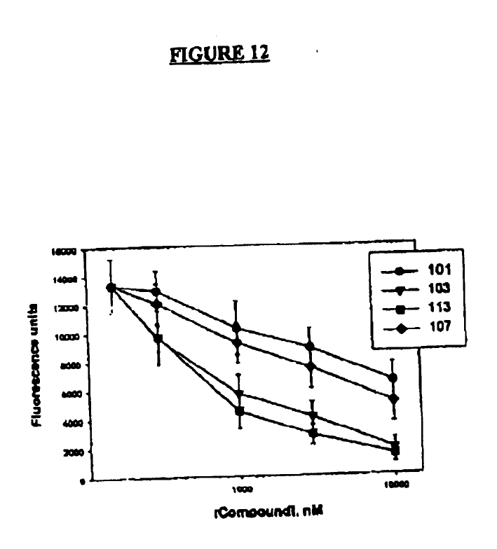

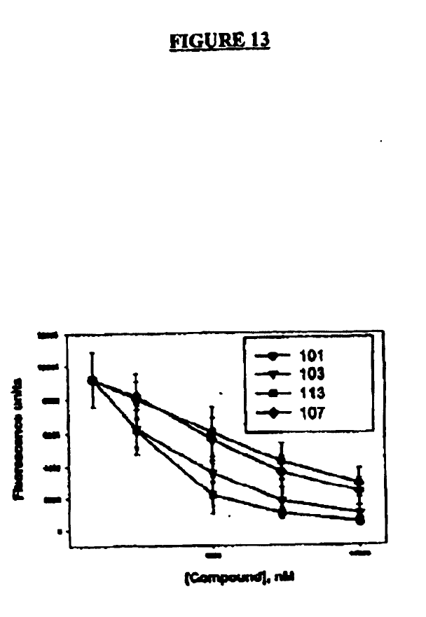

Species specificity was tested for compounds 101, 103, 107, and 113. FIGS. 11, 12 and 13 show the dose dependent inhibition of LPA-induced calcium mobilization by these compounds in HTC rat hepatoma cells transfected with the human Edg-4 receptor (FIG. 11), rat (FIG. 12) or mouse (FIG. 13) Edg-4 receptor. It should be noted that the HTC cells expressing human Edg 4 is a clonal cell line obtained by limiting dilution, while the rat and mouse cell lines are “pooled” populations of cells potentially containing cells that are untransfected. Under these circumstances, it would be expected that the compounds would not be as effective as in the clonal cell line, as is the case in this instance. However, the data demonstrate that the compounds have a similar inhibition profile against the rat and mouse Edg-4 receptor as compared with the human Edg-4 receptor.

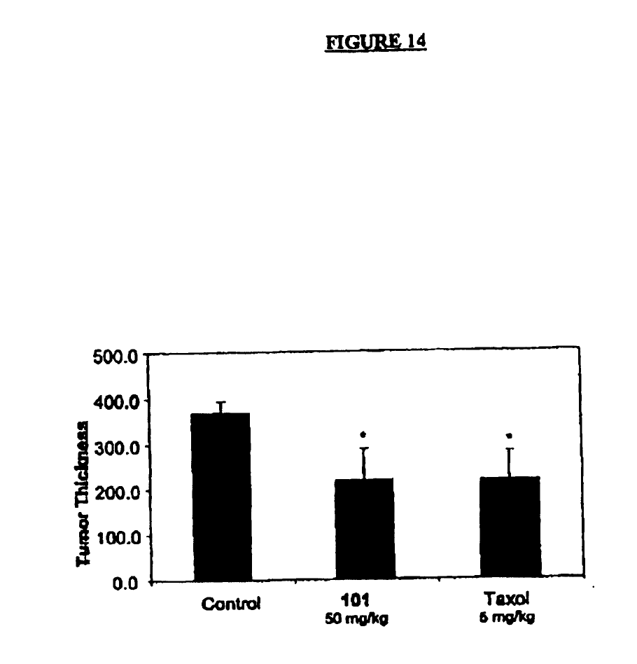

Compound 101 was tested in vivo in a Z chamber assay. In this assay (described below), 50 mg/kg of 101 significantly inhibited human ovarian tumor growth within the chamber. This level of inhibition was equivalent to inhibition of tumor growth seen with 5 mg/kg Taxol administered every other day (FIG. 14).

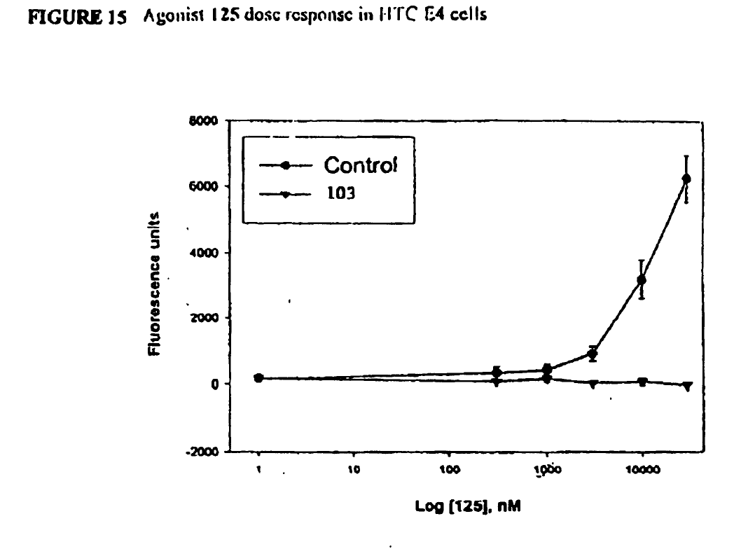

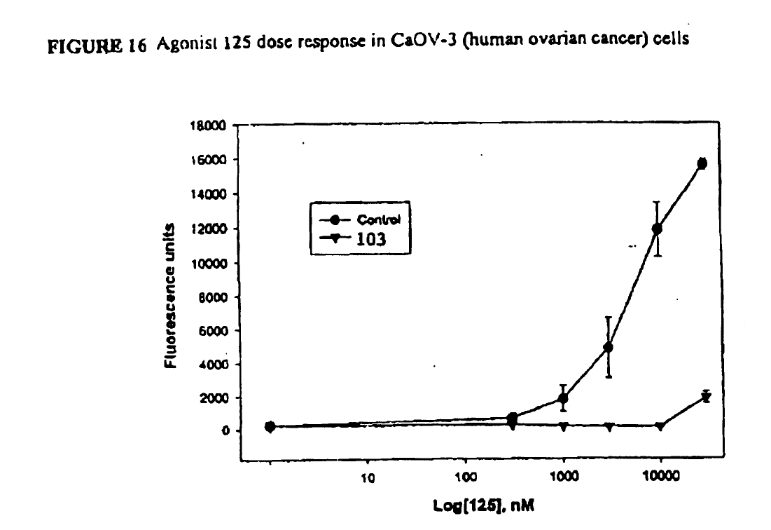

The Edg-4 agonist 125 elicits a calcium response in HTC rat hepatoma cells transfected with the human Edg-4 receptor, which is inhibited by the selective Edg-4 antagonist 103 (FIG. 15). This specificity is also observed in human ovarian cancer cells, CaOV3, which naturally express human Edg-4 receptor (FIG. 16). The selectivity of 125 is illustrated in Table 3, above.

### 6.26. Example 26

Intracellular Calcium Measurement Assays

LPA receptors such as Edg-4, couple to calcium effector pathways, and result in increases in intracellular calcium following receptor activation (An et al., Molecular Pharmacology, 54:881-888, 1998, incorporated herein by reference). This biological response lends itself to a very efficient, high-throughput screen using a Fluorescence Imaging Plate Reader (FLIPR; Molecular Devices, Sunnyvale, Calif.). The FLIPR system is a real-time, cell-based assay system with continuous fluorescence detection using a cooled CCD camera. The FLIPR system was used to developing an Edg-4 receptor screen. Rat hepatoma cells stably expressing Edg-4 receptor were plated on 384-well plates and loaded with a calcium dye loading kit (Molecular Devices, Sunnyvale, Calif.) for 1 hour at room temperature. Cells were then placed on the FLIPR³⁵⁴ (Molecular Devices, Sunnyvale, Calif.) and excited by an argon laser at 488 nm. The data for the entire 384-well plate was updated every second. An integrated robotic pipettor allowed for simultaneous compound addition into each individual well in the plate.

### 6.27. Example 27

IL-8 and VEGF Assays

IL-8 and VEGF assays were performed by standard enzyme-linked immunosorbent assay (“ELISA”) techniques. Cells were cultured in a 96 well format, serum starved overnight, and treated with LPA or S IP (doses range from 0.1-10 μM in serum free medium) for 24 hours. Cell supernatants were then collected to measure the amount of IL-8 secreted.

The assay was a standard sandwich ELISA in which an anti-IL-8 or VEGF capture antibody was adsorbed to a plastic dish. Cell supernatants containing IL-8 or VEGF were added to the dish, and then an anti-IL-8NVEGF biotinylated detection antibody and streptavidin-HRP were added.

Detection was via the addition of a substrate solution and colorimetric reading using a microtiter plate reader. The level of IL-8 or VEGF was interpolated by nonlinear regression analysis from a standard curve.

All reagents were from R&D Systems, Minneapolis, Minn.: MAB208 and AF-293-NA (capture antibody for IL-8 and VEGF respectively), BAF208 and BAF-293 (detection Ab for IL-8 and VEGF respectively), 208-IIL-010 and 293-VE-010 (recombinant human IL-8 protein standard and recombinant human VEGF protein standard respectively), DY998 (streptavidin-HRP), DY999 (substrate solution).

### 6.28. Example 28

Migration and Invasion Assays

Cells were plated in a 24 well format using Fluoroblok filter insert plates (8 μM pore size) or Fluoroblok matrigel coated filter insert plates (Becton Dickinson, San Diego, Calif.). The assay was a modified Boyden Chamber assay in which a cell suspension (1 ×10⁵ cells/ml) was prepared in serum free medium and added to the top chamber. LPA or SIP (doses ranged from 0.1-10 μM in serum free medium) was added to the bottom chamber. Following a 20-24 hour incubation period, the number of cells migrating or invading into the lower chamber was quantitated by transferring the filter insert into a fresh 24-well plate containing 4 μg/ml calcein AM (Molecular Probes, Sunnyvale, Calif.) in Hank's Balanced Salt Solution and staining for one hour.

Detection was via fluorescent readout at 450 nm excitation/530 nm emission using a fluorimeter. The level of fluorescence correlated with cell number.

For most cells types, no further manipulation was required. For CaOV3 human ovarian cancer cells, however, it was necessary that the cells be serum starved overnight prior to preparing the cell suspension. In addition, the filter inserts were coated with a solution of 1 mg/ml rat-tail Collagen I (BD, SanDiego, Calif.).

### 6.29. Example 29

Proliferation Assay

Cells were plated in a 96 well format. Treatments were performed directly without any serum starvation, and typically included LPA or SIP doses in a range from 0.1-10 μM in serum free medium. Cells were treated for 24-48 before the extent of cellular proliferation was measured.

The assay was performed using the ViaLight HS kit from BioWhittaker, Rockland, Me., which is based upon the bioluminescent measurement of ATP that is present in all metabolically active cells. The reaction utilized an enzyme, luciferase, which catalyzes the formation of light from ATP and luciferin. The emitted light intensity was linearly related to the ATP concentration, which correlated with cell number.

Measurement of cell proliferation required the extraction of ATP by the addition of Nucleotide Releasing Reagent, followed by the addition of the ATP Monitoring Reagent (both provided in kit). Detection was via chemiluminescence using the EG&G Berthold Luminometer, Gaithersburg, Md.

### 6.30. Example 30

cAMP Assay

Cells were plated in a 96 well format. Treatments were performed directly without any serum starvation. The cells were treated with forskolin to induce cAMP production, followed by LPA or S I P doses in the range from 0.1-10 μM in serum free medium. Following a 30-minute incubation period, the cells were lysed and the level of cAMP was determined.

The cAMP assay was performed using the Tropix cAMP-Screen (Applied BioSystems, Foster City, Calif.). The screen is a competitive immunoassay that utilizes a 96 well assay plate precoated with an anti-cAMP antibody. Cell lysates were added to the precoated plate, along with a cAMP-AP conjugate and a secondary anti-cAMP antibody.

Detection was performed using a substrate solution and chemiluminescent readout. The level of chemiluminescence was inversely proportional to the level of cAMP and was calculated from a standard curve.

### 6.31. Example 31

Pharmacology Profiling (Selectivity Assays)

In order to test the selectivity of compounds, various enzyme assays as well as radioligand binding assays were performed using numerous non-Edg receptor targets as listed below.

Enzyme Assays

1. Phosphodiesterase PDE1: (Nicholson et al., 1991, Trends Pharmacol. Sci. 12:19-27).

Source: Bovine heart

Substrate: 1.01 KM [³H]cAMP+cAMP

Vehicle: 1% DMSO

Pre-Incubation Time/Temp: None;

Incubation Time/Temp: 20 minutes at 25 ° C.

Incubation Buffer: 50 mM Tris-HCl, 5 mM MgCl2, 2 mM CaCl2, 10 unit Calmodulin, pH 7.5

Quantitation Method: Quantitation of [³H]adenosine

Significance Criteria: ≧50% of max stimulation or inhibition

2. Phosphodiesterase PDE2: (Nicholson et al., 1991, Trends Pharmnacol. Sci. 12:19-27).

Source: Human platelets

Substrate: 25.1 μM [³H]cAMP+cAMP

Vehicle: 1% DMSO

Pre-Incubation Time/Temp: None

Incubation Time/Temp: 20 minutes at 25 ° C.

Incubation Buffer: 50 mM Tris-HCl, 5 mM MgCl₂, pH 7.5

Quantitation Method: Quantitation of [³H]adenosine

Significance Criteria: >50% of max stimulation or inhibition

3. Phosphodiesterase PDE3: (Nicholson et al., 1991, Trends Pharmacol. Sci. 12:19-27).

Source: Human platelets

Substrate: 1.01 [μM [³H]cAMP+cAMP

Vehicle: 1 % DMSO

Pre-Incubation Time/Temp: None

Incubation Time/Temp: 20 minutes at 25 ° C.

Incubation Buffer: 50 mM Tris-HCl, 5 mM MgCl2, pH 7.5

Quantitation Method: Quantitation of [³H]adenosine

Significance Criteria: >50% of max stimulation or inhibition

4. Phosphodiesterase PDE4: (Cortijo et al., 1993)

Source: Human U937 cells

Substrate: 1.01 μM {³H]cAMP+cAMP

Vehicle: 1% DMSO

Pre-Incubation Time/Temp: None

Incubation Time/Temp: 20 minutes at 25 ° C.

Incubation Buffer: 50 mM Tris-HCl, 5 mM MgCl2, pH 7.5

Quantitation Method: Quantitation of [³H]adenosine

Significance Criteria: a 50% of max stimulation or inhibition

5. Phosphodiesterase PDE5: (Nicholson et al., 1991, Trends Pharmacol. Sci. 12:19-27).

Source: Human platelets

Substrate: 100 μM [³H]cGMP+cGMP

Vehicle: 1% DMSO

Pre-Incubation Time/Temp: None

Incubation Time/Temp: 20 minutes at 25 ° C.

Incubation Buffer: 50 mM Tris-HCl, 5 mM MgCl2, pH 7.5

Quantitation Method: Quantitation of [³H]guanosine

Significance Criteria: a 50% of max stimulation or inhibition

6. Phosphodiesterase PDE6: (Gillespie and Beavo, 1989)

Source: Bovine retinal rod outer segments

Substrate: 100 μM [³H]cGMP+cGMP

Vehicle: 1% DMSO

Pre-Incubation Time/Temp: None

Incubation Time/Temp: 20 minutes at 25° C.

Incubation Buffer: 50 mM Tris-HCl, 5 mM MgCl2, pH 7.5

Quantitation Method: Quantitation of [³H]gnanosine

Significance Criteria: >50% of max stimulation or inhibition

7. Phospholipase PLA₂-I (Katsumata et al.,1986, Anal. Biochem., 154:676-681).

Source: Porcine pancreas

Substrate: 0.03 μCi 1 -Palmitoyl-2-{1-¹⁴C]oleoyl-3-phosphatidylcholine

Vehicle: 1% DMSO

Pre-Incubation Time/Temp: 5 minutes at 37 ° C.

Incubation Time/Temp: 5 minutes at 37° C.

Incubation Buffer: 0.1 M glycine-NaOH, 20 M EDTA, pH 9.0

Quantitation Method: Quantitation of [¹⁴C]oleate

Significance Criteria: >50% of max stimulation or inhibition

8. Phospholipase PLA₂-II (Katsumata et al., 1986, Anal. Biochem. 154:676-681).

Source: Crotalus atrox

Substrate: 0.03 μCi 1 -Palmitoyl-2-[1-¹⁴C]oleoyl-3-phosphatidylcholine

Vehicle: 1% DMSO

Pre-Incubation Time/Temp: 5 minutes at 37° C.

Incubation Time/Temp: 5 minutes at 37° C.

Incubation Buffer: 0.1M glycine-NaOH, 20 M EDTA, pH 9.0

Quantitation Method: Quantitation of [¹⁴C]oleate

Significance Criteria: >50% of max stimulation or inhibition

9. Phospholipase PLC (Hergenrother et al, 1995, Anal. Biochem. 229:313-316).

Source: Bacillus cereus

Substrate: 400 μM 1,2-Dihexanoyl sn-glycerol-3-phosphocholine

Vehicle: 1% DMSO

Pre-Incubation Time/Temp: 10 minutes at 37° C.

Incubation Time/Temp: 5 minutes at 37 ° C.

Incubation Buffer: 0.1 M 3,3-dimethylglutaric acid, pH 7.3

Quantitation Method: Spectrophotometric quantitation of phosphorylcholine

Radioligand Binding Assays:

1. Adenosine A₁ (Liebert et al., 1992, Biochem. Biophys. Res. Commun. 187:919-926).

Source: Human recombinant CHO cells

Ligand: 1 nM ³H DPCPX

Vehicle: 0.4% DMSO

Incubation Time/Temp: 90 minutes at 25° C.

Incubation Buffer: 20 mM HEPES pH 7.4, 10 mM MgCl₂, 100 mM NaCl

NonSpecific Ligand: 100 μM R(-)-PIA

K_{d}: 1.4 nM*

Bₘₐₓ: 2.7 pmol/mg Protein*

Specific Binding: 85% *

Quantitation Method: Radioligand Binding

Significance Criteria: O 50% of max stimulation or

Quantitation Method: Radioligand Binding inhibition

Significance Criteria: ≧50% of max stimulation or inhibition

2. Adenosine A_{2A} (Varani et al., 1996, Br. J Pharmacol. 117:1693-1701)

Source: Human recombinant HEK-293 cells

Ligand: 0.05 μM ³H CGS-21680

Vehicle: 0.4% DMSO

Incubation Time/Temp: 90 minutes at 25 ° C.

Incubation Buffer: 50 mM Tris-HCl, pH 7.4. 10 mM MgCl₂, 1 mM EDTA, 2 U/mL adenosine deaminase

NonSpecific Ligand: 50 μM NECA

K_{d}: 0.064 μM *

Bₘₐₓ: 7 pmol/mg Protein*

Specific Binding: 85% *

Quantitation Method: Radioligand Binding

Significance Criteria: >50% of max stimulation or inhibition

3. Adrenergic α_{1A} (Michel et al., 1989, Br. J Pharmacol. 98:883-889).

Source: Wistar Rat submaxillary gland

Ligand: 0.25 nM ³H Prazosin

Vehicle: 0.4% DMSO

Incubation Time/Temp: 60 minutes at 25 ° C.

Incubation Buffer: 50 mM Tris-HCl, 0.5 mM EDTA, pH 7.4

NonSpecific Ligand: 10 μM Phentolamine

K_{d}: 0.17 nM*

Bₘₐₓ: 0.18 pmol/mg Protein*

Specific Binding: 90% *

Quantitation Method: Radioligand Binding

Significance Criteria: ≧50% of max stimulation or inhibition

4. Adrenergic α_{1A} (Uhlcn et al., 1994, J. Pharmacol. Exp. Ther. 271:1558)

Source: Human recombinant insect Sf9 cells

Ligand: 1 nM ³H MK-912

Vehicle: 0.4% DMSO

Incubation Time/Temp: 60 minutes at 25 ° C.

Incubation Buffer: 75 mM Tris-HCl, pH 7.4, 12.5 mM MgCl₂, 2 mM EDTA

NonSpecific Ligand: 10 μM WB-4 101

K_{d}: 0.06 nM*

Bₘₐₓ: 4.6 pmollmg Protein*

Specific Binding: 95% *

Quantitation Method: Radioligand Binding

Significance Criteria: ≧50% of max stimulation or inhibition

5. Adrenergic β₁, (Feve et al., 1994, Proc. Natl. Acad. Sci. USA 91:5677 5681)

Source: Human recombinant Rex 16 cells

Ligand: 0.3 nM 1251 Cyanopindolol

Vehicle: 0.4% DMSO

Incubation Time/Temp: 2 hours at 25 ° C.

Incubation Buffer: 50 mM Tris-HCl, 5 mM EDTA, 1.5 mM CaCl₂, 120 Mm NaCl, 1.4 mM ascorbic acid, 10 mg/L BSA, pH 7.4

NonSpecific Ligand: 100 gM S(-)-Propranolol

K_{d}: 0.041 nM *

Bₘₐₓ: 0.072 pmol/mg Protein*

Specific Binding: 95% *

Quantitation Method: Radioligand Binding

Significance Criteria: >50% of max stimulation or inhibition

6. Adrenergic β2 (McCrea and Hill, 1993, Brit. J Pharmacol. 110:619-626).

Source: Human recombinant CHO-NBR1 cells

Ligand: 0.2 nM ³H CCGP-12177

Vehicle: 0.4% DMSO

Incubation Time/Temp: 60 minutes at 25 ° C.

Incubation Buffer: 50 mM Tris-HCl, 0.5 mM EDTA, 5.0 mM MgCl₂, 120 mM NaCl, pH 7.4

NonSpecific Ligand: 10 μM ICI-118551

K_{d}: 0.44 nM*

Bₘₐₓ: 0.437 pmol/mg Protein*

Specific Binding: 95% *

Quantitation Method: Radioligand Binding

Significance Criteria: ≧50% of max stimulation or inhibition

7. Adrenergic, Norepinephrine Transporter (Galli et al., 1995, J. Exp. Biol. 198:2197-2212).

Source: Human recombinant MDCK cells

Ligand: 0.2 nM ¹²⁵I RTI-55

Vehicle: 0.4% DMSO

Incubation Time/Temp: 3 hours at 4° C.

Incubation Buffer: 50 mM Tris-HCl, 100 mM NaCl, 1 μM leupeptin, 10 μM PMSF, pH 7.4

NonSpecific Ligand: 10 μM Desipramine

K_{d}: 0.024 μM *

Bₘₐₓ: 2.5 pmol/mg Protein*

Specific Binding: 75% *

Quantitation Method: Radioligand Binding

Significance Criteria: >50% of max stimulation or inhibition

8. Calcium Channel Type L, Dihydropyridine (Ehlert et al., 1982, Life Sci. 30:2191-2202).

Source: Wistar Rat cerebral cortex

Ligand: 0.1 nM ³H Nitrendipine

Vehicle: 0.4% DMSO

Incubation Time/Temp: 90 minutes at 25 ° C.

Incubation Buffer: 50 mM Tris-HCl, pH 7.7

NonSpecific Ligand: 1 μM Nitrendipine

K_{d}: 0.18nM*

Bₘₐₓ 0.23 pmol/mg Protein*

Specific Binding: 91% *

Quantitation Method: Radioligand Binding

Significance Criteria: ≧50% of max stimulation or inhibition

9. Cannabinoid CB₁ (Felder et al., 1995, Mol. Pharmacol. 48:443-450).

Source: Human recombinant HEK-293 cells

Ligand: 8 nM ³H WIN-55,212-2

Vehicle: 0.4% DMSO

Incubation Time/Temp: 90 minutes at 37° C.

Incubation Buffer: 50 mM Hepes, pH 7.0, 5 mg/mL BSA

NonSpecific Ligand: 10 μM WIN-55,212-2

K_{d}: 0.3 μM *

Bₘₐₓ: 2.4 pmol/mg Protein*

Specific Binding: 70% *

Quantitation Method: Radioligand Binding

Significance Criteria: ≧50% of max stimulation or inhibition

10. Dopamine D₁ (Dearry et al., 1990, Nature 347:72-76).

Source: Human recombinant CHO cells

Ligand: 1.4 nM ³H SCH-23390

Vehicle: 0.4% DMSO

Incubation Time/Temp: 2 hours at 37° C.

Incubation Buffer: 50mM Tris-HCl, pH 7.4, 150 mM NaCl, 1.4 mM ascorbic acid, 0.001% BSA

NonSpecific Ligand: 10 μM (+)-Butaclamol

K_{d}: 1.4 nM*

Bₘₐₓ: 0.63 pmol/mg Protein*

Specific Binding: 95% *

Quantitation Method: Radioligand Binding

Significance Criteria: ≧50% of max stimulation or inhibition

11. Dopamine D2L (Bunzo et al., 1988, Nature 336:783-787).

Source: Human recombinant CHO cells

Ligand: 0.16 μM ³H Spiperone

Vehicle: 0.4% DMSO

Incubation Time/Temp: 2 hours at 25 ° C.

Incubation Buffer: 50 mM Tris-HCl, pH 7.4, 150 mM NaCl, 1.4mM ascorbic acid, 0.00 1% BSA

NonSpecific Ligand: 10 μM Haloperidol

K_{d}: 0.08 nM*

Bₘₐₓ: 0.48 pmol/mg Protein*

Specific Binding: 85% *

Quantitation Method: Radioligand Binding

Significance Criteria: ≧50% of max stimulation or inhibition

12. GABA_{A}, Agonist Site (Enna and Snyder, 1976, Mol Pharmacol. 13:442-453).

Source: Wistar Rat brain (minus cerebellum)

Ligand: 1 nM ³H Muscimol

Vehicle: 0.4% DMSO

Incubation Time/Temp: 10 minutes at 4° C.

Incubation Buffer: 50 mM Tris-HCl, pH 7.4

NonSpecific Ligand: 0.1 μM Muscimol

K_{d}: 3.8 nM*

Bₘₐₓ: 1.8 pmol/mg Protein*

Specific Binding: 90% *

Quantitation Method: Radioligand Binding

Significance Criteria: ≧50% of max stimulation or inhibition

13. Glutamate, NMDA, Phencyclidine (Goldman et al., 1985, FEBS Lett. 190:333-336).

Source: Wistar Rat cerebral cortex

Ligand: 2 nM ³H Idazoxan

Vehicle: 0.4% DMSO

Incubation Time/Temp: 30 minutes at 25° C.

Incubation Buffer: 50 mM Tris-HCl, 0.5 mM EDTA, pH 7.4

NonSpecific Ligand: 0.1 μM MK-801 (Dizolcipine)

K_{d}: 4 M*

Bₘₐₓ: 0.78 pmol/mg Protein*

Specific Binding: 94% *

Quantitation Method: Radioligand Binding

Significance Criteria: ≧50% of max stimulation or inhibition

14. Histamine H₁, Central (Hill et al., 1978, J. Neurochem. 31:997-1004).

Source: Guinea pig cerebellum

Ligand: 1.75 nM ³H Pyrilamine

Vehicle: 0.4% DMSO

Incubation Time/Temp: 60 minutes at 25 ° C.

Incubation Buffer: 50 mM K-Na phosphate buffer pH 7.4 at 25° C.

NonSpecific Ligand: 1 μM Pyrilamine

K_{d}: 0.23 μM *

Bₘₐₓ: 0.198 pmol/mg Protein*

Specific Binding: 90% *

Quantitation Method: Radioligand Binding

Significance Criteria: >50% of max stimulation or inhibition

15. Imidazoline 12, Central (Brown et al., 1990, Br. J Pharmacol. 99:803-809).

Source: Wistar Rat cerebral cortex

Ligand: 2 nM ³H Idazoxan

Vehicle: 0.4% DMSO

Incubation Time/Temp: 30 minutes at 25 ° C.

Incubation Buffer: 50 mM Tris-HCl, 0.5 mM EDTA, pH 7.4 at 25° C.

NonSpecific Ligand: 1 μM Idazoxan

K_{d}: 4nM*

Bₘₐₓ: 0.14 pmol/mg Protein*

Specific Binding: 85% *

Quantitation Method: Radioligand Binding

Significance Criteria: ≧50% of max stimulation or inhibition

16. Muscarinic M₂ (Delmendo et al., 1989, Br. J Pharmacol. 96:457-464).

Source: Human recombinant insect Sf9 cells

Ligand: 0.29 nM ³H N-Methylscopolamine (NMS)

Vehicle: 0.4% DMSO

Incubation Time/Temp: 60 minutes at 25 ° C.

Incubation Buffer: 50 mM Tris-HCl, pH 7.4 10 mM MgCl₂, 1 mM EDTA

NonSpecific Ligand: 1 μM Atropine

K_{d}: 0.16 nM*

Bₘₐₓ: 4.9 pmol/mg Protein*

Specific Binding: 96% *

Quantitation Method: Radioligand Binding

Significance Criteria: ≧50% of max stimulation or inhibition

17. Nicotinic Acetylcholine, Central (Pabreza et al., 1991, Mol. Pharmacol. 39:9-12).

Source: Wistar Rat brain

Ligand: 2 nM ³H Cytisine

Vehicle: 0.4% DMSO

Incubation Time/Temp: 75 minutes at 4° C.

Incubation Buffer: 50 mM Tris-HCl, 120 mM NaCl, 5mM KCl, 1 mMMgCl₂, 2.5 mM CaCl₂, pH 7.4

NonSpecific Ligand: 100 μM Nicotine

K_{d}: 1 nM *

Bₘₐₓ: 0.026 pmol/mg Protein*

Specific Binding: 90% *

Quantitation Method: Radioligand Binding

Significance Criteria: ≧50% of max stimulation or inhibition

18. Opiate μ (Wang et al., 1994, FEBS Lett. 338:217-222).

Source: Human recombinant CHO-K1 cells

Ligand: 0.6 nM ³H Diprenorphine

Vehicle: 0.4% DMSO

Incubation Time/Temp: 60 minutes at 25 ° C.

Incubation Buffer: 50 mM Tris-HCl, pH 7.4

NonSpecific Ligand: 10 μM Naloxone

K_{d}: 0.41 μM*

Bₘₐₓ: 3.8 pmol/mg Protein*

Specific Binding: 90% *

Quantitation Method: Radioligand Binding

Significance Criteria: ≧50% of max stimulation or inhibition

19. Phorbol Ester (Ashendel, 1985, Biochem. Biophys. Acta 822:219-242).

Source: ICR Mouse brain

Ligand: 3 nM ³H PDBu

Vehicle: 0.4% DMSO

Incubation Time/Temp: 60 minutes at 25 ° C.

Incubation Buffer: 20 mM Tris-HCl, containing 5 mM CaCl₂, pH 7.5 at 25° C.

NonSpecific Ligand: 1 M PDBu

K_{d}: 8.7 nM*

Bₘₐₓ: 26 pmol/mg Protein*

Specific Binding: 80% *

Quantitation Method: Radioligand Binding

Significance Criteria: ≧50% of max stimulation or inhibition

20. Platelet-Derived Growth Factor (PDGF) (Williams et al., 1984, J. Biol. Chem. 259:5287-5294).

Source: Mouse 3T3 cells

Ligand: 0.02 nM ¹²⁵I PDGF

Vehicle: 0.4% DMSO

Incubation Time/Temp: 45 minutes at 25° C.

Incubation Buffer: HBSS, 2 mg/ml BSA, 1 mM MgCl₂, 1 mM CaCl₂

NonSpecific Ligand: 0.1 nM PDGF

K_{d}: 0.012 nM*

Bₘₐₓ: 3100 R/cell Receptor/cell*

Specific Binding: 88% *

Quantitation Method: Radioligand Binding

Significance Criteria: ≧50% of max stimulation or inhibition

21. Potassium Channel [K_{ATP}] (Gaines et al., 1988, J Biol. Chem. 263:2589-2592).

Source: Syrian hamster pancreatic beta cells HIT-T15

Ligand: 5 nM ³H Glyburide

Vehicle: 0.4% DMSO

Incubation Time/Temp: 2 hours at 25° C.

Incubation Buffer: 50 mM MOPS, 0.1 mM CaCl₂, pH 7.4

NonSpecific Ligand: 10 μM Glyburide

K_{d}: 0.64nM*

Bₘₐₓ: 1 pmol/mg Protein*

Specific Binding: 90% *

Quantitation Method: Radioligand Binding

Significance Criteria: ≧50% of max stimulation or inhibition

22. Sigma σ₁ (Ganapathy et al., 1999, Pharmacol. Exp. Ther. 289:251-260).

Source: Human Jurkat cells TEB-152

Ligand: 8 nM ³H Haloperidol

Vehicle: 0.4 % DMSO

Incubation Time/Temp: 4 hours at 25° C.

Incubation Buffer: 5 mM K₂HPO₄/KH₂PO₄ buffer pH 7.5

NonSpecific Ligand: 10 μM Haloperidol

K_{d}: 5.8nM*

Bₘₐₓ: 0.71 pmol/mg Protein*

Specific Binding: 80% *

Quantitation Method: Radioligand Binding

Significance Criteria: ≧50% of max stimulation or inhibition

23. Sigma σ₂ (Hashimoto and London, 1993, Eur. J Pharmacol. 236:159-163

Source: Wistar Rat brain

Ligand: 3 nM ³H Ifenprodil

Vehicle: 0.4% DMSO

Incubation Time/Temp: 60 minutes at 37° C.

Incubation Buffer: 50 mM Tris-HCl, pH 7.4

NonSpecific Ligand: 10 μM Ifenprodil

K_{d}: 4.8 nM*

Bₘₐₓ: 1.3 pmol/mg Protein *

Specific Binding: 85% *

Quantitation Method: Radioligand Binding

Significance Criteria: ≧50% of max stimulation or inhibition

24. Sodium Channel, Site 2 (Catterall et al., 1981, J. Biol. Chem. 256:8922-8927.

Source: Wistar Rat brain

Ligand: 1.5 nM ₃H Batrachotoxinin A 20-μ-Benzoate

Vehicle: 0.4% DMSO

Incubation Time/Temp: 30 minutes at 37° C.

Incubation Buffer: 50 mM Tris-HCl, pH 7.4 at 25° C., 50 mM Hepes, 130 mM choline-Cl, 5.4 mM KCl, 0.8 mM MgSO₄.7H₂O, 5.5 mM glucose, 40 μg/ml LqTx

NonSpecific Ligand: 100 μM Veratridine

K_{d}: 0.013 RM *

Bₘₐₓ: 0.88 pmol/mg Protein *

Specific Binding: 85% *

Quantitation Method: Radioligand Binding

Significance Criteria: ≧50% of max stimulation or inhibition

25. Vascular Endothelial Growth Factor (VEGF) (Gitay-Goren et al., 1996, J. Biol. Chem. 271:5519-5523).

Source: Human umbilical vein endothelial cells

Ligand: 0.1 μM ¹²⁵I VEGF₁₆₅

Vehicle: 0.4% DMSO

Incubation Time/Temp: 3 hours at 25° C.

Incubation Buffer: Buffer 1: M199 medium, 20% FBS, 100 U/ml penicillin and 100 μg/ml streptomycin, 4 mM L-glutamate, 15 mM Hepes, pH 7.4.

Buffer 2: Buffer 1 containing 1 μg/ml Heparmn and 0.1% gelatin

NonSpecific Ligand: 3 nM VEGF₁₆₅

K_{d}: 0.035 nM *

Bₘₐₓ: 8900 R/cell Receoptors/cell*

Specific Binding: 85% *

Quantitation Method: Radioligand Binding

Significance Criteria: ≧50% of max stimulation or inhibition

* Historical Values

6.32. Example 31

In Vivo Z-Chamber Study

The Z-chamber assay is a fibrin-based in vivo assay, wherein fibrin and thrombin are added through a port in a two-sided chamber sealed by a nylon mesh. The chamber is implanted in the subcutaneous space of an animal and harvested for evaluation. Fibrin matrices are formed in normal wound healing and are used by tumors to sustain growth, thus Z-chambers are designed to study, for example, angiogenesis, wound healing, and tumor growth. In addition, their design is useful, for example, in studies of localized gene expression, stem cell, adenoviral, and tissue generation.

The efficacy of 101 in an in vivo tumor model was examined by Z-chamber® (SRI, Menlo Park, Calif.) study. Each tumor chamber consisted of 150-160 μl cell suspension made by suspending 190 million cells in 20 ml fibrin (4 mg/ml). Following the introduction of cell suspension, 2 units of thrombin was added into each chamber and the mixture was allowed to gel for 5-7 minutes before implantation.

Rats were anesthetized with Nembutal (35 mg/kg). The skin of rats was surgically prepared with 70% alcohol. Two incisions (approximately 2 cm in length) were made on the back, one over the mid vertebral and the other over the lower vertebral region. Pockets were made in the subcutaneous fascia lateral to the incisions by blunt dissection with the help of scissors, and the chambers were placed deep into these pockets. The incision wounds were later closed with an autoclip stapling device.

101 was dissolved in a 1 to 1 cremophor:ethanol solution and diluted 4 times with 5% dextrose on water. Animals with tumor chambers were injected daily with 50 mg/kg 101 or 5 mg/kg taxol every other day as a positive control. Tumor chambers were harvested on day 16 post implantation. Chambers were cleared of all fascia, and tissue in each chamber was fixed in 10% formalin, paraffin embedded and stained with hematoxylin and eosin. The tumor thickness was measured and compared to a negative control, i.e., chambers with no treatment, and the positive treatment. Four rats were used per each group. The results are summarized in FIG. 14.

These studies demonstrate the efficacy of illustrative compounds of the invention in reducing tumor thickness in an in vivo model. Particularly, illustrative compounds of the invention are effective in modulating biological activities of Edg-4, for example, inhibiting cell proliferation in an in vivo model.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims.

All publications and patents cited herein are incorporated by reference in their entirety.

1. A method of modulating an Edg-4 receptor mediated biological activity comprising contacting a cell expressing the Edg4 receptor with an amount of a modulator of the Edg-4 receptor sufficient to modulate the Edg4 receptor mediated biological activity wherein the modulator is not a phospholipid.

2. A method of modulating an Edg4 receptor mediated biological activity in a subject comprising administering to the subject a therapeutically effective amount of a modulator of the Edg-4 receptor wherein the modulator is not a phospholipid.

3. The method of claim 1 or 2, wherein the modulator is an agonist.

4. The method of claim 1 or 2, wherein the modulator is an antagonist.

5. The method of claim 1 or 2, wherein the modulator exhibits at least about 200 fold inhibitor selectivity for Edg4 relative to other Edg receptors.

6. The method of claim 1 or 2, wherein the modulator exhibits at least about 10 fold inhibitory selectivity for Edg-4 relative to other Edg receptors.

7. The method of claim 1 or 2, wherein the modulator exhibits at least about 200 fold inhibitory selectivity for Edg4 relative to Edg-2 and Edg-7 receptors.

8. The method of claim 1 or 2, wherein the modulator exhibits at least about 10 fold inhibitory selectivity for Edg-4 relative to Edg-2 and Edg-7 receptors.

9. The method of claim 1 or 2, wherein the biological activity is cell proliferation.

10. The method of claim 9, wherein the modulator exhibits at least about 200 fold inhibitory selectivity for Edg-4 relative to other Edg receptors.

11. The method of claim 9, wherein the modulator exhibits at least about 10 fold inhibitory selectivity for Edg-4 relative to other Edg receptors.

12. The method of claim 9, wherein the modulator exhibits at least about 200 fold inhibitory selectivity for Edg-4 relative to Edg2 and Edg-7 receptors.

13. The method of claim 9, wherein the modulator exhibits at least about 10 fold inhibitory selectivity for Edg-4 relative to Edg-2 and Edg-7 receptors.

14. The method of claim 9, wherein cell proliferation leads to ovarian cancer, peritoneal cancer, endometrial cancer, cervical cancer, breast cancer, colon cancer or prostrate cancer.

15. The method of claim 9, wherein cell proliferation is stimulated by LPA.

16. The method of claim 1 or 2, wherein the biological activity is calcium mobilization, VEGP synthesis, IL-8 synthesis, platelet activation, cell migration, phosphoinositide hydrolysis, inhibition of cAMP formation, increasing the level of fatty acids, actin polymerization, apoptosis, angiogenesis, inhibition of wound healing, inflammation, expression of endogenous protein growth factors, cancer invasiveness, regulation of autoimmunity or atherogenesis.

17. The method of claim 1 or 2 wherein the modulator binds to the Edg-4 receptor with a binding constant of at least about 1 μM.

18. The method of claim 1 or 2 wherein the modulator binds to the Edg-4 receptor with a binding constant between about 1 μM and 100 nM.

19. The method of claim 1 or 2, wherein the modulator is a nucleic acid, peptide or carbohydrate.

20. The method of claim 1 or 2, wherein the modulator is an organic molecule of molecular weight of less than 750 daltons.

21. The method of claim 1, wherein the cell is a HTC hepatoma cell, an ovarian cell, an epithelial cell, a fibroblast cell, a neuronal cell, a Xenopus laevis oocyte cell, a carcinoma cell, a pheochromocytoma cell, a myoblast cell, a platelet cell or a fibrosarcoma cell.

22. The method of claim 21, wherein the cell is OV202 human ovarian cell, a HTC rat hepatoma cell, SKOV3 and CAOV-3 human ovarian cancer cells, MDA-MB-453 breast cancer cell, MDA-MB-23 1 breast cancer cell, HUVEC cells A43 1 human epitheloid carcinoma cell or a HT-1 080 human fibrosarcoma cell.

23. The method of claim 1 or 2, wherein the modulator is a compound of stuctural formula (I): 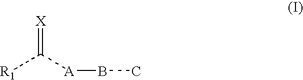
or a pharmaceutically available solvate or hydrate thereof, wherein:
R₁ is hydrogen, alkyl, substituted alkyl, acylamino, substituted acylamino, alkylamino, substituted alkylamino, alkylthio, substituted alkylthio, alkoxy, substituted alkoxy, alkylarylamino, substituted alkylarylamino, amino, arylalkyloxy, substituted arylalkyloxy, aryl, substituted aryl, arylamino, substituted arylamino, arylalkyl, substituted arylalkyl, dialkylamino, substituted dialkylamino, cycloalkyl, substituted cycloalkyl, cycloheteroalkyl, substituted cycloheteroalkyl, heteroaryloxy, substituted heteroaryloxy, heteroaryl, substituted heteroaryl, heteroalkyl, substituted heteroalkyl sulfonylamino or substituted sulfonylamino;
X═O or S;
A is NR₂, O or S;
R₂ is hydrogen, alkyl or substituted alkyl; and
B and C are independently alkyl, substituted alkyl, aryl, substituted aryl, heteroaryl, substituted heteroaryl, cycloalkyl, substituted cycloalkyl, cycloheteroalkyl or substituted cycloheteroalkyl

24. The method of claim 23, wherein R₁ is alkyl, substituted alkyl, aryl, substituted aryl, arylalkyloxy or substituted sulfonylamino.

25. The method of claim 23, wherein R₁ is substituted alkyl.

26. The method of claim 23, wherein R₁ is substituted haloalkyl.

27. The method of claim 23, wherein R₁ is substituted trifluoroalkyl.

28. The method of claim 23, wherein R₁ has the structural formula (II): 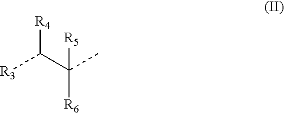
wherein:
R₃ is haloalkyl or substituted haloalkyl;
R₄ is oxo or thiono; and
R₅ and R₆ are independently hydrogen, halo, alkyl or substituted alkyl.

29. The method of claim 28, wherein R₃ is fluoroalkyl, R₄ is oxo and R₅ and R₆ are independently hydrogen, halo or alkyl.

30. The method of claim 28, wherein R₃ is trifluoromethyl, R₄ is oxo and R₅ and R₆ are independently hydrogen, chloro or methyl.

31. The method of claim 28, wherein R₅ and R₆ are hydrogen.

32. The method of claim 28 wherein R₅ is hydrogen and R₆ is chloro or methyl.

33. The method of claim 23, wherein X is 0, A is NR₂ and R₂ is hydrogen.

34. The method of claim 23, wherein B and C are independently, aryl, substituted aryl, heteroaryl or substituted heteroaryl.

35. The method of claim 23, wherein B and C are independently indolo, substituted indolo, imidazolo, substituted, imidazolo, pyrazolo, substituted pyrazolo, phenyl or substituted phenyl.

36. The method of claim 23, wherein B is heteroaryl or substituted heteroaryl and C is aryl or substituted aryl.

37. The method of claim 23, wherein B is pyrazolo or substituted pyrazolo and C is phenyl or substituted phenyl.

38. The method of claim 23, wherein the modulator is a compound of structural formula (III); 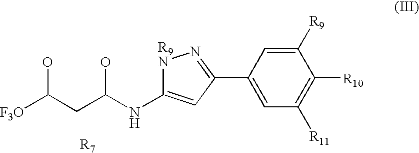
wherein:
R₇ is hydrogen, alkyl, substituted alkyl or halo;
R₈ is hydrogen, carbamoyl or substituted carbamoyl; and
R₉, R₁₀ and R₁₁ are independently hydrogen, alkoxy, substituted alkoxy, halo or P.₉ and P.₁₀ together with the carbons to which they are attached form a [1,3] dioxolane ring.

39. The method of claim 23, wherein the modulator is compound of the formula:

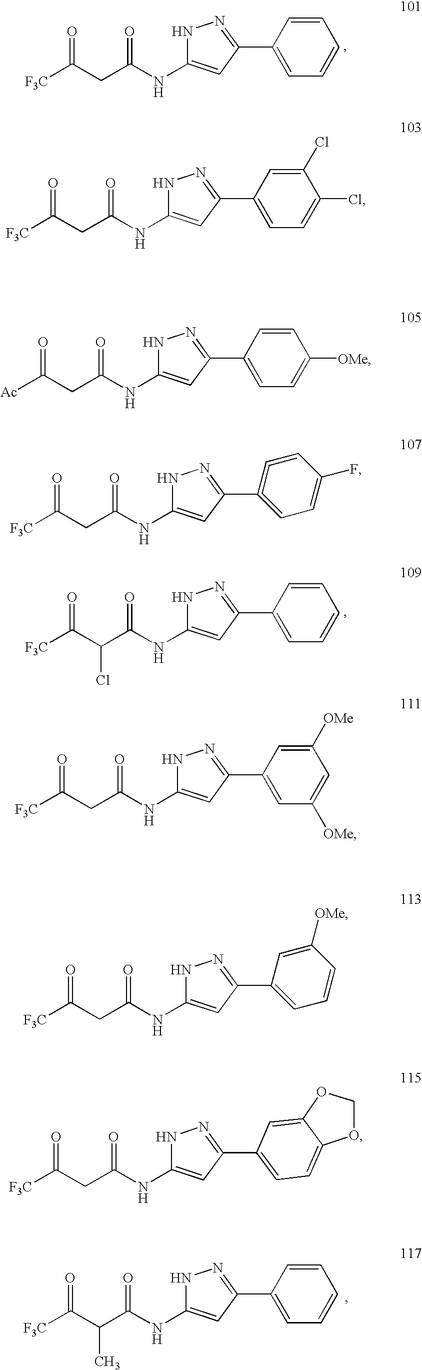

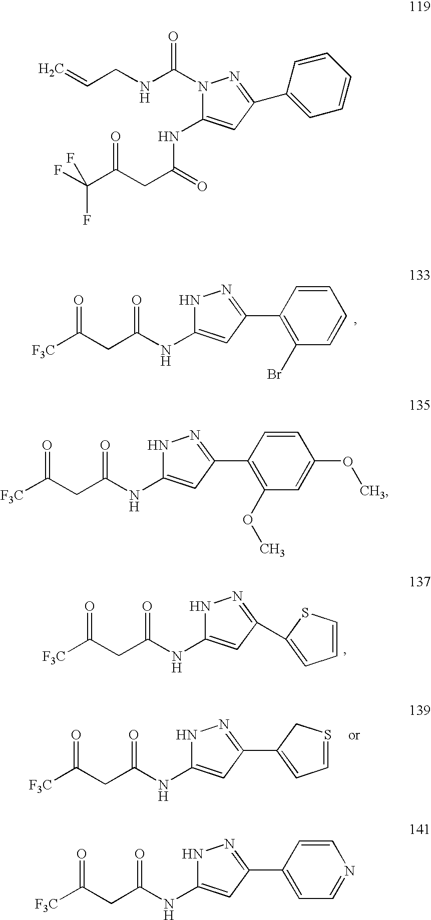

40. The method of claim 1 or 2, wherein the modulator is a compound of structural formula (IV): 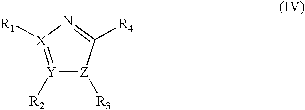
or a pharmaceutically available solvate or hydrate thereof, wherein;
each of R₁, R₂, R₃, R₄ or R₅ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(_{CH2})ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, —(C₅-C₁₀)cycloheteroaryl, —(C₃-C₆)cycloheteroalkyl, -naphthyl, —(C₃-C₁₀)heterocycle, —CO₂(CH₂)ₘR₅, —NHC(O)R₅, —NHC(O)OR₅, —NHC(O)NHR₅, —NR₅R₅, ═NR₅, —(C₁-C₁₀)alkylNHC(O)(CH₂)ₘR₅, —(C₃-C₁₀)cyloheteroalkyl(R₅)ₘ, —(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 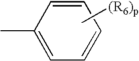
wherein;
each R₅ and R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or SO₂NH₂;
m is independently an integer ranging from 0 to 8;
p is independently an integer ranging from 0 to 5;
X and Y are each independently C or N; and
Z is O, S, C or N, wherein if Z is O or S, then R₃ is an electron pair;
R₁ and R₂ can optionally together form a 5-, 6-, or 7-membered substituted or unsubstituted yclic or aromatic ring;
R₂ and R₃ can optionally together form a 5-, 6-, or 7-membered substituted or unsubstituted yclic or aromatic ring; and
R₃ and R₄ can optionally together form a 5-, 6-, or 7-membered substituted or unsubstituted cyclic or aromatic ring.

41. The method of claim 40, wherein the modulator is a compound of the following formula:

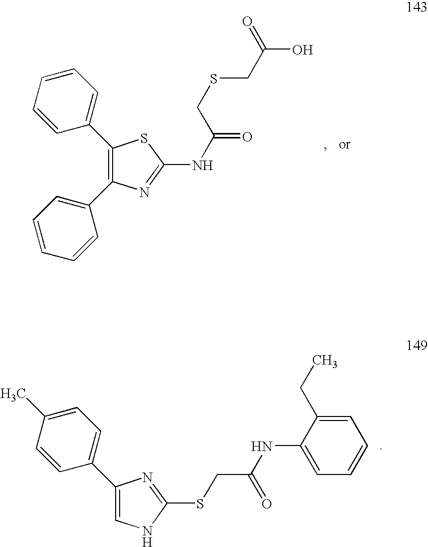

42. The method of claim 1 or 2, herein the modulator is a compound of structural formula (V): 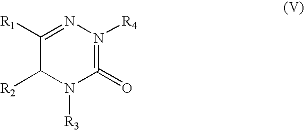
or a pharmaceutically available solvate or hydrate thereof, wherein:
each of R₁, R₂, R₃, R₄ or R₅ is independently —H, -halo, —NO₂, —CN, —OH, —N(R₅)(R₅), —O(CH₂)ₘR₅, —C(O)R₅, —C(O)NR₅R₅, —C(O)NN(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 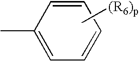
wherein;
each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)(C₁-C₁₀)alkyl, —OC(O)O(C₁-C₁₀)alkyl, or —SO₂NH₂;
m is independently an integer ranging from 0 to 8;
p is independently an integer ranging from 0 to 5; and
R₁ and R₂ or R₂ and R₃ can optionally together form a 5-, 6.-, or 7-membered substituted or unsubstituted cyclic or aromatic ring.

43. The method of claim 42, wherein R₁ and R₂ are independently aryl, substituted aryl, heteroaryl or substituted heteroaryl.

44. The method of claim 42, wherein R₂ is indole and R₃ and R₄ are hydrogen.

45. The method of claim 42, wherein the modulator is a compound of the following formula: 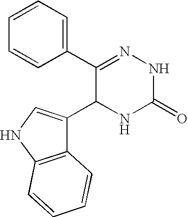
or its (+) and (−) enantiomers.

46. The method of claim 1 or 2, wherein the modulator is a compound of structural formula (V): 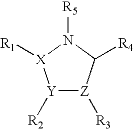
or a pharmaceutically available solvate or hydrate thereof, wherein:
each of R₁, R₂, R₃, R₄ or R₅ is independently —H, -halo, —NO₂, —CN, —OH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -naphthyl, —(C₃-C₁₀)heterocycle, —CO₂(CH₂)ₘR₅, —NHC(O)R₅, —NHC(O)OR₅, —NHC(O)NHR₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 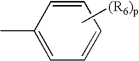
wherein;
R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂(CH₂)ₘH, —NHC(O)(C₁-C₁₀)alkyl, —NHC(O)NH(C₁-C₁₀)alkyl, —OC(O)(C₁-C₁₀)alkyl, —OC(O)O(C₁-C₁₀)alkyl, or —SO₂-NH₂;
m is independently an integer ranging from 0 to 8;
p is independently an integer ranging from 0 to 5;
X, Y and Z are independently O, S, C or N, wherein if X, Y or Z is O or S, R₁ is an electron pair;
R₁ and R₂ or can optionally together form a 5-, 6-, or 7-membered substituted or unsubstituted cyclic or aromatic ring;
R₃ and R₄ can optionally together form a 5-, 6- or 7-membered substituted or unsubstituted cyclic or aromatic ring;
R₁ and R₅ can optionally together form a 5-, 6- or 7-membered substituted or unsubstituted cyclic or aromatic ring; and
R₄ and R₅ can optionally together form a 5-, 6- or 7-membered substituted or unsubstituted cyclic or aromatic ring.

47. The method of claim 46, wherein R₁ and R₂ together form a 5-, 6- or 7-membered substituted or unsubstituted cyclic or aromatic ring.

48. The Method of claim 46, wherein: R₁ and R₂ together form a 5-, 6- or 7-membered substituted or unsubstituted cyclic or aromatic ring; and R₃ and R₄ together form a 5-, 6- or 7-membered substituted or unsubstituted cyclic or aromatic ring.

49. The method of claim 46, wherein: R₁ and R₂ together form a 6-membered substituted or unsubstituted cyclic or aromatic ring; and R₃ and R₄ together form a 6-membered substituted or unsubstituted cyclic or aromatic ring.

50. The method of claim 46, wherein: R₁ and R₂ form a 6-membered substituted cyclic or aromatic ring, and R₃ and R₄ form a 6-membered substituted cyclic or aromatic ring.

51. The method of claim 46, wherein the modulator is a compound of the following formula:

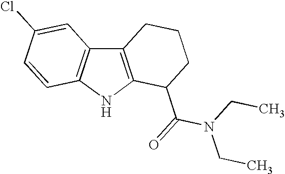

52. The method of claim 1 or 2, wherein the modulator is a compound of structural formula (VII): 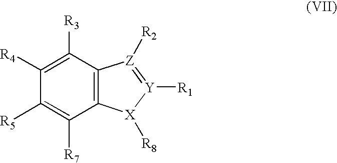
or a pharmaceutically available solvate or hydrate thereof, wherein:
each of R₁, R₂, R₃, R₄, R₅, R₇ or R₈ is independently —H, -halo, —NO₂, —CN, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 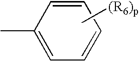
wherein:
each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₃-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;
m is independently an integer ranging from 0 to 8;
p is independently an integer ranging from 0 to 5;
X is O, S, C or N, wherein if X is O or S, R₁ is an electron pair; and
Y and Z are independently N or C, wherein if Y or Z is N, R₁ and R₂ are each an electron pair.

53. The method of claim 52, wherein the modulator is a compound of the following formula:

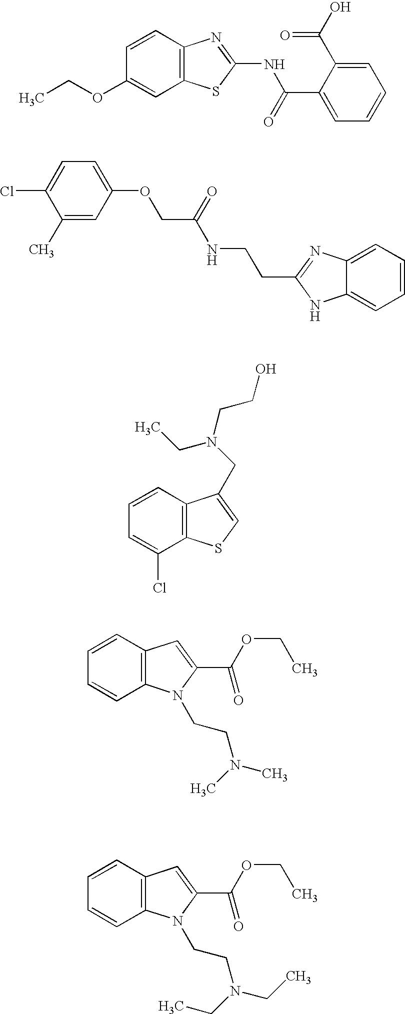

54. The method of claim 1 or 2, wherein the modulator is a compound of structural formula (VIII): 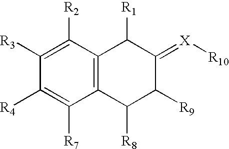
or a pharmaceutically available solvate or hydrate thereof, wherein:
each of R₁, R₂, R₃, R₄, R₅, R₇, R₈, R₉ or R₁₀ is independently —H, -halo, —NO₂, —CN, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 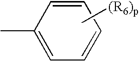
wherein;
each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloakyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;
m is independently an integer ranging from 0 to 8;
p is independently an integer ranging from 0 to 5; and
X and Y are independently O, S or N, wherein if X or Y is O or S, R₉ and R₁₀ are an electron pair.

55. The method of claim 54, wherein R₇ is substituted or unsubstituted aryl.

56. The method of claim 54, wherein the modulator is a compound of the following formula:

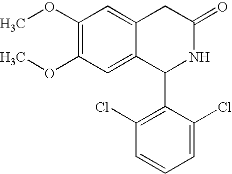

57. The method of claim 1 or 2, wherein the modulator is a compound of structural formula (IX): 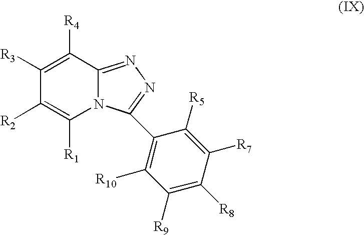
or a pharmaceutically available solvate or hydrate thereof, wherein:
each of R₁, R₂, R₃, R₄, R₅, R₇, R₈, R₉ or R₁₀ is independently —H, -halo, —NO₂, —CN, —C(R₅)₃, —(CH₂)ₘOH, —N(R₅)(R₅), —O(CH₂)ₘR₅, —C(O)R₅, —C(O)NR₅R₅, —C(O)NH(CH₂)ₘ(R₅), —OCF₃, -benzyl, CO₂CH(R₅)(R₅), —(C₁-C₁₀)alkyl, —(C₂-C₁₀)alkenyl, —(C₂-C₁₀)alkynyl, (C₃-C₁₀)cycloalkyl, —(C₈-C₁₄)bicycloalkyl, —(C₅-C₁₀)cycloalkenyl, (C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-_{C10})alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 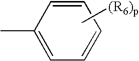
wherein;
each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;
m is independently an integer ranging from 0 to 8; and
p is independently an integer ranging from 0 to 5.

58. The method of claim 57, wherein R₂ is a substituted alkyl, and one or more of R₅, R₇, R₈, R₉ and R₁₀ are halos.

59. The method of claim 57, wherein R₂ is a halo-substituted alkyl.

60. The method of claim 57, wherein R₂ is —CF₃.

61. The method of claim 57, wherein the modulator is a compound of the following formula:

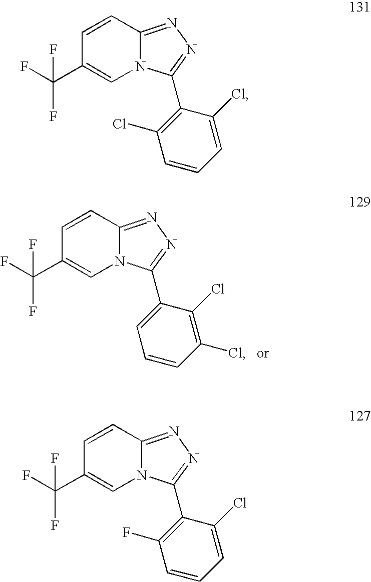

62. The method of claim 1 or 2, wherein the modulator is a compound of structural formula (X): 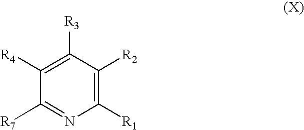
or a pharmaceutically available solvate or hydrate thereof, wherein:
each of R₁, R₂, R₃, R₄, R₅ or R₇ is independently —H, -halo, —NO₂, —CN, —C(R₅)₃, —(CH₂)ₘOH, —N(R₅)(R₅), —O(CH₂)ₘR₅, —C(O)R₅, —C(O)NR₅R₅, —C(O)NH(CH₂)ₘ(R₅), —OCF), -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, -naphthyl, —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)CH₂)ₘR₅, —(C₁-C₁₀)alkylNR₅R₅, —CO₂H, —(C₁-C₁₀)alkylC(O)NH(CH₂)ₘR₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 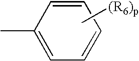
wherein;
each R₅ or R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO(CH₂)ₘH, —NHC(O)(C₁-C₁₀))alkyl, —NHC(O)NH(C₁-C₁₀)alkyl, —OC(O)O(C₁-C₁₀)alkyl, or —SO₂NH₂;
m is independently an integer ranging from 0 to 8;
p is independently an integer ranging from 0 to 5;
R₁ and R₂ can optionally together form a 5-, 6- or 7-membered substituted or unsubstituted cyclic or aromatic ring;
R₂ and R₃ can optionally together form a 5-, 6- or 7-membered substituted or unsubstituted cyclic or aromatic ring;
R₃ and R₄ can optionally together form a 5-, 6- or 7-membered substituted or unsubstituted cyclic or aromatic ring; and
R₄ and R₇ can optionally together form a 5-, 6- or 7-membered substituted or unsubstituted cyclic or aromatic ring.

63. The method of claim 62, wherein R₃ and R₇ are substituted or unsubstituted aryls.

64. The method of claim 62, wherein the modulator is a compound of the following formula:

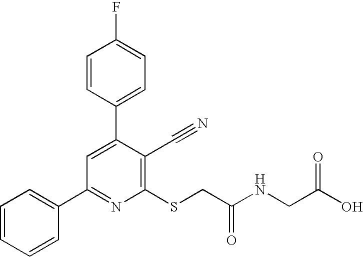

65. The method of claim 1 or 2, wherein the modulator is a compound of structural formula (XI): 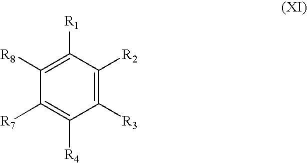
or a pharmaceutically available solvate or hydrate thereof, wherein;
each of R₁, R₂, R₃, R₄, R₅, R₇ or R₈ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(CH₂)ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(Cₓ-C₁₄)bicycloalkyl, —(C₅-C₁₀)cycloalkenyl, —(C₅)heteroaryl, —(C₆)heteroaryl, —(C₅-C₁₀)heteroaryl, —(C₅-C₁₀)cycloheteroaryl, -naphthyl —(C₃-C₁₀)heterocycle, —CO₂(CH₂)ₘR₅, —NHC(O)R₅, —NHC(O)OR₅, —NHC(O)NHR₅, —(C₁-C₁₀)alkylNHC(O)(CH₂)ₘR₅, —(C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 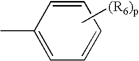
wherein;
each R₆ is independently -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;
m is independently an integer ranging from 0 to 8;
p is independently an integer ranging from 0 to 5;
R₁ and R₂ can optionally together form a 5-, 6- or 7-membered substituted or unsubstituted cyclic or aromatic ring;
R₂ and R₃ can optionally together form a 5-, 6- or 7-membered substituted or unsubstituted cyclic or aromatic ring;
R_{J} and R₄ can optionally together form a 5-, 6- or 7-membered substituted or unsubstituted cyclic or aromatic ring;
R₄ and R₇ can optionally together form a 5-, 6- or 7-membered substituted or unsubstituted cyclic or aromatic ring;
R₇ and R₈ can optionally together form a 5-, 6- or 7-membered substituted or unsubstituted cyclic or aromatic ring; and
R₁ and R₈ can optionally together form a 5- 6- or 7-membered substituted or unsubstituted cyclic or aromatic ring.

66. The method of claim 65, wherein R₂ and R₃ together form a 5-membered ring.

67. The method of claim 65, wherein R₂ and R₃ together form a 5-membered ring, and R₇ and R₈ together form a 5-membered ring.

68. The method of claim 65, wherein the modulator is a compound of the following formula:

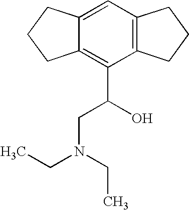

69. The method of claim 65, wherein R₂ is a substituted or unsubstituted pipeline moiety.

70. The method of claim 65, wherein the modulator is a compound of the following formula:

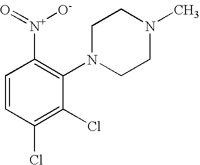

71. The method of claim 1 or 2, wherein the modulator is a compound of structural formula (XII): 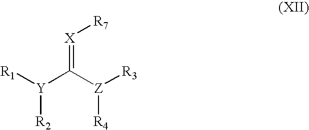
or a pharmaceutically available solvate or hydrate thereof, wherein;
each of R₁, R₂, R₃, R₄, R₅ or R₇ is independently —H, -halo, —NO₂, —CN, —C(R₅)₃, —(CH₂)ₘOH, —(CH₂)ₘN(R₅)(R₅), —O(CH₂)ₘR₅, —C(O)R₅, —C(O)NR₅R₅, —C(O)NH(CH₂)ₘ(R₅), —C(OH)R₅, —OCF₃, -benzyl, —CO₂CH(R₅)(R₅), —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, —(C₅-C₁₀)heteroaryl, —(C₅-C₁₀)cycloheteroaryl, —(C₃-C₆)cycloheteroalkyl, -naphthyl, —(C₃-C₁₀)heterocycle, —CO₂(CH₂)ₘR₅, —NHC(O)R₅, NHC(O)OR₅, —NHC(O)NHR₅, —NR₅R₅, ═NR₅, —(C₁-C₁₀)alkylNHC(O)(CH₂)ₘR₅, —(C₃-C₁₀)cycloheteroalkyl(R₅)ₘ, —(CH₂)ₘR₅, —C₁-C₁₀)alkylNR₅R₅, —OC(O)(CH₂)ₘCHR₅R₅, —CO₂(CH₂)ₘCHR₅R₅, —OC(O)OR₅, —SR₅, —S(O)R₅, —S(O)₂R₅, —S(O)₂NHR₅, or 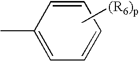
wherein;
each R₅ or R₆ is independently —H, -halo, —NO₂, —CN, —OH, —CO₂H, —N(C₁-C₁₀)alkyl(C₁-C₁₀)alkyl, —O(C₁-C₁₀)alkyl, —C(O)(C₁-C₁₀)alkyl, —C(O)NH(CH₂)ₘ(C₁-C₁₀)alkyl, —OCF₃, -benzyl, —CO₂(CH₂)ₘCH((C₁-C₁₀)alkyl(C₁-C₁₀)alkyl), —CO₂(C₁-C₁₀)alkyl, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —(C₅)heteroaryl, —(C₆)heteroaryl, -phenyl, naphthyl, —(C₃-C₁₀)heterocycle, —CO₂(CH₂)ₘ(C₁-C₁₀)alkyl, —CO₂(CH₂)ₘH, —NHC(O)(C₁-C₁₀)alkyl, —NHC(O)NH(C₁-C₁₀)alkyl, —OC(O)O(C₁-C₁₀)alkyl, or —SO₂NH₂;
m is independently an integer ranging from 0 to 8;
p is independently an integer ranging from 0 to 5;
₃ or R₄ can optionally form a substituted or unsubstituted cyclic, aromatic, heterocyclic, heteroaryl or cycloheteroalkyl ring;
R₁ or R₂ can optionally form a substituted or unsubstituted cyclic, aromatic, heterocyclic, heteroaryl or cycloheteroalkyl ring; and
R₂ or R₄ can optionally form a substituted or unsubstituted cyclic, aromatic, heterocyclic, heteroaryl or cycloheteroalkyl ring.

72. The method of claim 71, wherein the modulator is a compound of the following formula:

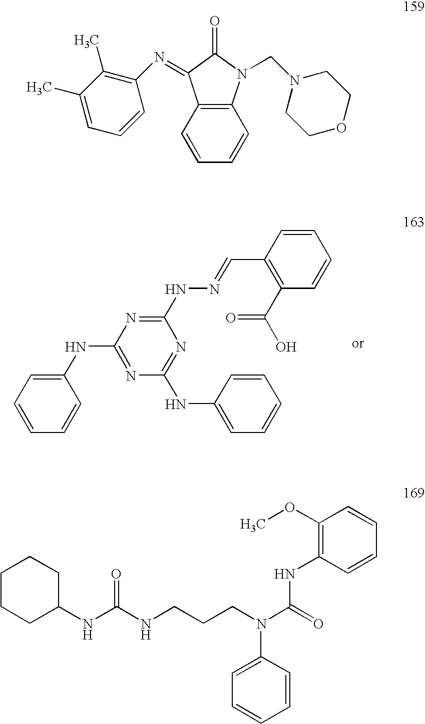

73. A method for treating or preventing cancers, acute lung diseases, acute inflammatory exacerbation of chronic lung diseases, surface epithelial cell injury, or cardiovascular diseases in a patient comprising administering to a patient in need of such treatment or prevention a therapeutically effective amount of a compound of structural formula (I)-(XII).

74. A method for treating or preventing ovarian cancer, peritoneal cancer, endometrial cancer, cervical cancer, breast cancer, colorectal cancer, uterine cancer, stomach cancer, small intestine cancer, thyroid cancer, lung cancer, kidney cancer, pancreas cancer, prostrate cancer, adult respiratory distress syndrome (ARDS), asthma, transcomcal freezing, cutaneous burns, ischemia or arthesclerosis in a patient comprising administering to a patient in need of such treatment or prevention a therapeutically effective amount of a compound of structural formula (I)-(XII).

75. A method for treating or preventing cancers, acute lung diseases, acute inflammatory exacerbation of chronic lung diseases, surface epithelial cell injury, or cardiovascular diseases in a patient comprising administering to a patient in need of such treatment or prevention a therapeutically effective amount of a compound of structural formula (I)-(XII) and one or more agonists or antagonists of an LPA receptor.

76. A method for treating or preventing cancers, acute lung diseases, acute inflammatory exacerbation of chronic lung diseases, surface epithelial cell injury, or cardiovascular diseases in a patient comprising administering to a patient in need of such treatment or prevention a therapeutically effective amount of a compound of structural formula (I)-(XII) and one or more drugs useful in treating or preventing cancers, acute lung diseases, acute inflammatory exacerbation of chronic lung diseases, surface epithelial cell injury, or cardiovascular diseases.
